(19) United States
(12) Patent Application Publication
(10) Pub. No.: US 2025/0049899 A1
(43) Pub. Date: Feb. 13, 2025

(54) IMMUNOGENIC FUSION PROTEIN COMPOSITIONS AND METHODS OF USE THEREOF

(71) Applicant: Matrivax, Inc., Boston, MA (US)

(72) Inventors: Enda MORAN, Boston, MA (US); Robert Thompson CARTEE, Kingston, MA (US); Kevin P. KILLEEN, Needham, MA (US)

(21) Appl. No.: 18/711,368

(22) PCT Filed: Nov. 18, 2022
(86) PCT No.: PCT/US2022/080168

§ 371 Date: May 17, 2024

Related U.S. Application Data
(60) Provisional application No. 63/280,908, filed on Nov. 18, 2021.

(51) Int. Cl. A61K 39/00; A61P 37/04; C07K 14/315

(52) U.S. Cl. CPC A61K 39/0006; A61P 37/04; C07K 14/3156; C07K 2319/40

(57) ABSTRACT

The disclosure relates to immunogenic fusion protein compositions and methods of use thereof for preventing or treating pneumococcal infection. The disclosure relates to methods of producing and purifying immunogenic fusion proteins. This disclosure further relates to compositions and formulations comprising immunogenic fusion proteins.

### RELATED APPLICATIONS

This application claims priority to, and the benefit of, U.S. Provisional Application No. 63/280,908, filed Nov. 18, 2021. The contents of this application are incorporated herein by reference in its entirety.

### FIELD OF THE INVENTION

The present invention relates to the field of vaccines for preventing or treating pneumococcal infection.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “MTRV-001_001WO_Seq_Listing_ST26.xml”. The XML file is 65,890 bytes in size, created on Nov. 18, 2022.

### BACKGROUND

Streptococcus pneumoniae is a Gram positive bacterium which is a major cause of disease such as sepsis, meningitis, otitis media and lobar pneumonia (Tuomanen et al. NEJM 322:1280-1284, 1995). Infection by S. pneumoniae remains a significant health threat worldwide. Pneumococci bind avidly to cells of the upper and lower respiratory tract and to endothelial cells present in blood vessels. Like most bacteria, adherence of pneumococci to human cells is achieved by presentation of bacterial surface factors that bind to eukaryotic cell surface proteins (Cundell, D. & Tuomanen, E. (1994) Microb Pathog 17:361-374). For example, bacteria translocate across cells of the upper respiratory tract and nasopharynx via the polymeric immunoglobulin receptor (pIgR) (Zhang et al. (2000) Cell 102:827-837). Alternatively, when the bacteria are in the blood stream, the pneumococcal bacteria bind to endothelial cells, and the bacteria cross the blood vessel endothelium and enter tissues by binding to and transcytosing with the platelet activating factor (PAF) receptor (Cundell et al. (1995) Nature, 377:435-438).

Current vaccines against S. pneumoniae employ purified carbohydrates of the capsules of up to the 23 most common serotypes of this bacterium found in disease, however unconjugated polysaccharide vaccines are only 50% protective against pneumonia (Shapiro et al. NJEM 325:1453, 1991) and are not immunogenic in children under the age of 2. Conjugate vaccines against S. pneumoniae involve the covalent linkage of pneumococcal capsular polysaccharides to proteins such as diphtheria toxoid or tetanus toxoid in order elicit higher immune responses and provide protection in children under 2 years of age. The protection against pneumococcal disease including pneumonia, sepsis, or meningitis provided by these vaccines, however, is limited to the serotypes present in the formulation, thereby leaving patients unprotected against most of the greater than one-hundred S. pneumoniae serotypes. Further, vaccines that can prevent colonization of the nasopharynx, tissue invasion, and disease symptoms of S. pneumoniae regardless of serotype are needed in the art. Therefore, compositions and methods provided herein fills these needs by providing pharmaceutical compositions (e.g., vaccines) for the prevention and treatment of a wide range of serotypes of pneumococcal infections across all age groups.

### SUMMARY OF THE INVENTION

The present disclosure provides an immunogenic fusion protein comprising an amino acid sequence of SEQ ID NO: 43. The present disclosure provides a polynucleotide encoding any one of the immunogenic fusion proteins of the disclosure. The present disclosure provides a host cell comprising any one of the polynucleotides of the disclosure.

The present disclosure provides a composition comprising any one of the immunogenic fusion proteins of the disclosure and a pharmaceutically acceptable carrier.

In some embodiments, the immunogenic fusion protein is glycosylated. In some embodiments, the immunogenic fusion protein is not glycosylated.

In some embodiments, the composition further comprises at least one adjuvant. In some embodiments, the adjuvant comprises aluminum hydroxide, aluminum phosphate or aluminum sulfate. In some embodiments, the adjuvant comprises aluminum hydroxide. In some embodiments, the aluminum hydroxide comprises Alhydrogel®.

The present disclosure provides i) a population of purified immunogenic fusion proteins, wherein at least about 90% of the purified immunogenic fusion proteins are full-length purified immunogenic fusion proteins comprising the amino acid sequence of SEQ ID NO: 43; ii) less than 80,000 ng of host cell protein/mg of purified immunogenic fusion protein; and/or iii) less than 17 EU of endotoxin/mg of purified immunogenic fusion protein.

In some embodiments, the composition comprises: i) a population of purified immunogenic fusion proteins, wherein about 95%, about 96%, about 97%, about 98% or about 99% of the purified immunogenic fusion proteins are full-length purified immunogenic fusion proteins comprising the amino acid sequence of SEQ ID NO: 43; ii) less than 50 ng of host cell protein/mg of purified immunogenic fusion protein; and/or iii) less than 2 EU of endotoxin/mg of purified immunogenic fusion protein.

The present disclosure provides a method of producing an immunogenic fusion protein, comprising the steps of: a) culturing a population of the host cells expressing an immunogenic fusion protein comprising the amino acid sequence of SEQ ID NO: 43 in a condition suitable for the population of host cells to produce the immunogenic fusion protein; b) disrupting the cell membranes of the host cells; c) recovering a sample comprising the immunogenic fusion protein and one or more impurities; d) contacting the sample comprising the immunogenic fusion protein with a hydrophobic interaction chromatography resin and eluting the immunogenic fusion protein from the hydrophobic interaction chromatography resin under conditions that allow for preferential detachment of the immunogenic fusion protein, thereby obtaining an eluate comprising the immunogenic fusion protein; e) subjecting the eluate comprising the immunogenic fusion protein of step d) to a flow through anion exchange resin, thereby obtaining an eluate comprising the immunogenic fusion protein; and f) contacting the eluate comprising the immunogenic fusion protein of step e) with a multi-modal chromatography resin and eluting the immunogenic fusion protein from the multi-modal chromatography resin under conditions that allow for preferential detachment of the immunogenic fusion protein, thereby obtaining an eluate comprising the immunogenic fusion protein. In some embodiments, the host cell is an E. coli cell.

In some embodiments, the method further comprises the step of: g) contacting the eluate comprising the immunogenic fusion protein of step f) with a flow through anion exchange membrane; thereby obtaining an eluate comprising the immunogenic fusion protein. In some embodiments, the method further comprises the steps of: h) contacting the eluate comprising the immunogenic fusion protein of step g) with an ultrafiltration/diafiltration membrane; and i) washing the immunogenic fusion protein from the ultrafiltration/diafiltration membrane under conditions that allow for preferential detachment of the immunogenic fusion protein, thereby obtaining an eluate comprising the immunogenic fusion protein. In some embodiments, the method further comprises the step of: j) contacting the eluate comprising the immunogenic fusion protein of step i) with a 0.2 μm filter.

The present disclosure provides a composition comprising a purified immunogenic fusion protein produced by any one of the methods of the disclosure.

The present disclosure provides a formulation comprising: i) an immunogenic fusion protein comprising the amino acid sequence of SEQ ID NO: 43; ii) a surfactant; iii) a buffer; and iv) a salt. In some embodiments, the surfactant is at a concentration of about 175 μg/mL to about 375 μg/mL.

In some embodiments, i) the immunogenic fusion protein is at a concentration of about 0.5 mg/mL to about 1.5 mg/mL; ii) the surfactant is at a concentration of about 175 μg/mL to about 375 μg/mL; iii) the buffer is at a concentration of about 5 mM to about 20 mM; iv) the salt is at a concentration of about 50 mM to about 200 mM; and wherein the pH level of the formulation is between pH 6 and pH 9.

In some embodiments, i) the immunogenic fusion protein is at a concentration of about 0.8 mg/mL to about 1.2 mg/mL; ii) the surfactant is at a concentration of about 275 μg/mL; iii) the buffer is at a concentration of about 10 mM; iv) the salt is at a concentration of about 154 mM; and wherein the pH level of the formulation is about 7.4.

In some embodiments, the buffer comprises sodium phosphate, the salt comprises sodium chloride (NaCl) and/or the surfactant comprises polysorbate 20.

In some embodiments, the formulation further comprises an adjuvant. In some embodiments, the adjuvant is at a concentration of about 0.5 mg/mL to about 2 mg/mL. In some embodiments, the adjuvant is at a concentration of about 1 mg/mL. In some embodiments, the adjuvant is selected from the group consisting of aluminum hydroxide, aluminum phosphate and aluminum sulfate. In some embodiments, the adjuvant is aluminum hydroxide. In some embodiments, the aluminum hydroxide is Alhydrogel®.

The present disclosure provides a formulation comprising: about 1.0 mg/mL of an immunogenic fusion protein comprising the amino acid sequence of SEQ ID NO: 43, about 275 μg/mL polysorbate 20, about 10 mM sodium phosphate and about 154 mM sodium chloride, and wherein the pH level of the formulation is about 7.4.

The present disclosure provides a formulation comprising: about 20 μg/mL of an immunogenic fusion protein comprising the amino acid sequence of SEQ ID NO: 43, about 275 μg/mL polysorbate 20, about 1 mg/mL of aluminum hydroxide in 9 mM of sodium phosphate and about 139 mM sodium chloride, and wherein the pH level of the formulation is about 7.4.

The present disclosure provides a formulation comprising: about 60 μg/mL of an immunogenic fusion protein comprising the amino acid sequence of SEQ ID NO: 43, about 275 μg/mL polysorbate 20, about 1 mg/mL of aluminum hydroxide in 9 mM of sodium phosphate and about 139 mM sodium chloride, and wherein the pH level of the formulation is about 7.4.

The present disclosure provides a formulation comprising: about 120 μg/mL of an immunogenic fusion protein comprising the amino acid sequence of SEQ ID NO: 43, about 275 μg/mL polysorbate 20, about 1 mg/mL of aluminum hydroxide in 9 mM of sodium phosphate and about 139 mM sodium chloride, and wherein the pH level of the formulation is about 7.4.

The present disclosure provides a formulation comprising: about 180 μg/mL of an immunogenic fusion protein comprising the amino acid sequence of SEQ ID NO: 43, about 275 μg/mL polysorbate 20, about 1 mg/mL of aluminum hydroxide in 9 mM of sodium phosphate and about 139 mM sodium chloride, and wherein the pH level of the formulation is about 7.4.

The present disclosure provides a method of inducing a protective immune response in a subject comprising administering to the subject, any one of the compositions or any one of the formulations of the disclosure.

The present disclosure provides method of immunizing a subject against an infection caused by Streptococcus pneumoniae, the method comprising administering to the subject, any one of the compositions or any one of the formulations of the disclosure.

The present disclosure provides a method of treating, prophylactically preventing, or reducing the occurrence of a condition, disease, or infection caused by Streptococcus pneumoniae, in a subject in need thereof comprising administering to the subject, any one of the compositions or any one of the formulations of the disclosure.

In some embodiments, the subject is administered with at least one dose of the immunogenic fusion protein. In some embodiments, the subject is administered with no more than two doses of the immunogenic fusion protein. In some embodiments, the dose further comprises about 1 mg/mL of aluminum hydroxide.

In some embodiments, the dose comprises about 1 μg to about 150 μg of the immunogenic fusion protein. In some embodiments, the dose comprises about 10 μg of the immunogenic fusion protein. In some embodiments, the dose comprises about 30 μg of the immunogenic fusion protein. In some embodiments, the dose comprises about 60 μg of the immunogenic fusion protein. In some embodiments, the dose comprises about 90 μg of the immunogenic fusion protein.

In some embodiments, the amount of time between each dose is from about 4 weeks to about one year. In some embodiments, the amount of time between each dose is about one week, about two weeks, about three weeks or about four weeks. In some embodiments, the amount of time between each dose is about four weeks.

In some embodiments, the composition or the formulation is administered by parenteral administration. In some embodiments, the parenteral administration is by intramuscular injection.

In some embodiments, the subject is between 0 and 80 years of age. In some embodiments, the subject is between 0 and 2 years of age. In some embodiments, the subject is between 18 and 50 years of age. In some embodiments, the subject is between 60 and 75 years of age.

### BRIEF DESCRIPTION OF THE DRAWINGS

FIGS. 1A-1B are schematics depicting the structure and construction of MTRV001. FIG. 1A is a schematic depicting the structure of MTRV001 comprising PLY with two amino acid substitutions (G293S and L460D) and flanking CbpA fragments. CbpA: choline binding protein A; PLY: pneumolysin. FIG. 1B depicts the construction of MTRV001. CbpA: choline binding protein A; PLY: pneumolysin; PLY-DM: double-mutant pneumolysin; PLY-SM: single mutant pneumolysin; YLN: PLY-SM with flanking CbpA peptides.

FIGS. 2A-2B are schematics depicting the construction of CbpA peptides used in MTRV001. FIG. 2A is a schematic representation of the R2 domain of CbpA (left panel). Boxes identify two nonhelical loop regions and amino acid motifs. Amino acid numbers of the R2 domain are indicated. The percentage conservation of sequence in each motif from 30 clinical isolates is as shown (right panel). FIG. 2B is a schematic depicting regions of R2 that were expressed. Amino acid numbers of the R2 domain are indicated.

FIGS. 3A-3B are two survival curves depicting survival of MXV01 and PLY-DM immunized BALB/c mice following intranasal (IN) challenge with two dose levels of a serotype 19F S. pneumoniae strain. FIG. 3A shows a 1× challenge dose (6.81×10⁷ CFU per dose). FIG. 3B shows a 0.5× challenge dose (3.69×10⁷ CFU per dose). CFU; colony forming units.

FIGS. 4A-4B are two survival curves depicting survival of MXV01 and PLY-DM immunized BALB/c mice following intranasal (IN) challenge with two dose levels of a serotype 6B S. pneumoniae strain. FIG. 4A shows a 1× challenge dose (1.45×10⁸ CFU per dose). FIG. 4B shows a 0.5× challenge dose (4.45×10⁷ CFU per dose). CFU; colony forming units.

FIGS. 5A-5B are two survival curves depicting survival of MXV01 and PLY-DM immunized BALB/c mice following intranasal (IN) challenge with two dose levels of a serotype 22F S. pneumoniae strain. FIG. 5A shows a 1× challenge dose (1.19×10⁸ CFU per dose). FIG. 5B shows a 0.5× challenge dose (9.27×10⁷ CFU per dose). CFU; colony forming units.

FIGS. 6A-6B are two graphs depicting anti-PLY and anti-CbpA IgG titers from mice immunized with MTRV001, PLY-DM, or vehicle control. Antibody titers were determined by ELISA at day 42 (14 days following the third immunization). FIG. 6A shows anti-PLY IgG titers. FIG. 6B shows anti-CbpA IgG titers. CbpA: choline binding protein A; GMT: geometric mean titer; IgG: immunoglobulin G; PBS: phosphate-buffered saline; PLY-DM: pneumolysin double mutant.

FIG. 7 is a survival curve depicting survival of mice immunized with MTRV001, PLY-DM, and vehicle control following intratracheal (IT) infection with a virulent serotype 4 S. pneumoniae strain. PLY-DM: pneumolysin double mutant.

FIGS. 8A-8C are a series of microscopy images depicting lung histopathology of mice immunized with MTRV001, PLY-DM, and vehicle control 72 hours post-intratracheal (IT) challenge with virulent serotype 4 S. pneumoniae strain. FIG. 8A shows treatment with MTRV001. FIG. 8B shows PLY-DM treatment. FIG. 8C shows vehicle control (PBS) treatment. PBS: phosphate-buffered saline; PLY-DM: pneumolysin double mutant. All images are at original magnification 20× with hematoxylin and eosin staining.

FIG. 9 is diagram depicting the overall study schema of a Phase 1, First-in Human, Randomized, Double-Blind, Placebo Controlled, Dose-Escalation Study of the Tolerability, Safety, and Immunogenicity of MTRV001.

FIG. 10 is diagram depicting study intervention administration schema of a Phase 1, First-in Human, Randomized, Double-Blind, Placebo Controlled, Dose-Escalation Study of the Tolerability, Safety, and Immunogenicity of MTRV001.

FIG. 11 is diagram depicting participant timeline of a Phase 1, First-in Human, Randomized, Double-Blind, Placebo Controlled, Dose-Escalation Study of the Tolerability, Safety, and Immunogenicity of MTRV001.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Streptococcus pneumoniae (S. pneumoniae) is responsible for significant morbidity and mortality in pediatric, elderly, and immunocompromised populations across the world despite the availability of effective vaccines and antibiotics. It is one of the most common human bacterial pathogens and causes serious infections such as pneumonia, meningitis, and bacteremia as well as more common, but less severe, infections such as acute otitis media and sinusitis. S. pneumoniae is the leading cause of lower respiratory tract infection morbidity and mortality globally, and accounts for more deaths from pneumonia than all other causes, both viral and bacterial combined (GBD, 2018). Pneumococcal infections caused an estimated 740,000 deaths globally in children <5 years of age in 2019 (WHO, 2021) and S. pneumoniae is responsible for approximately 30% of all adult pneumonia cases in developed countries with a corresponding mortality rate of 11% to 40% (Daniels, 2016). S. pneumoniae remains a major cause of morbidity and death in the elderly, with people >65 years of age experiencing up to a 5-fold greater incidence of death due to community-acquired pneumonia compared to those <65 years of age (Adler, 2017). Due to emerging antibiotic resistance, inadequate protection of currently available polysaccharide-based vaccines, and limited vaccine accessibility in low- and lower middle-income countries, there remains a significant need to generate broadly protective, vaccines for preventing pneumococcal infections.

The S. pneumoniae polysaccharide capsule is an essential virulence factor that protects the pathogen from the host immune response, specifically complement mediated opsonophagocytosis (Goldblatt, 2008). Of importance, a robust antibody response to a specific capsular serotype confers significant protection against infection by S. pneumoniae expressing that particular capsular serotype.

At present, >100 serotypes of S. pneumoniae have been identified that vary in their monosaccharide composition and glycosidic bonds (Donati, 2010; Weinberger, 2011; Geno, 2015; Geno, 2017; GPSC, 2022). This remarkable diversity in S. pneumoniae capsular serotypes is in part due to the genetic structure of the capsule locus and the bacteria's natural competence for genetic transformation potentiating the generation of novel capsule types due to immune selective pressure.

The essential nature of the capsule for pneumococcal virulence, coupled with its surface localization and accessibility to antibodies is the basis of serotype specificity (Daniels, 2016). Moreover, these characteristics have targeted vaccine efforts to focus on the polysaccharide capsule. There are 2 general categories of commercialized pneumococcal vaccines to prevent S. pneumoniae infection. Although both categories are polysaccharide in nature, 1 type is based on polysaccharides alone (pneumococcal polysaccharide vaccine or PPV [e.g., PNEUMOVAX® 23]) and the other is based on polysaccharides conjugated to a protein carrier for enhanced immunogenicity (PCV or polysaccharide conjugate vaccine [e.g., Prevnar 20®]). Unlike PPVs, PCVs elicit a high-titer, anamnestic response, and IgA in the nasopharynx that reduces nasopharyngeal carriage and transmission of vaccine serotypes as well as confers a high level of efficacy (Orami, 2020).

PPVs consist of a mixture of unconjugated polysaccharides that are T-helper cell independent antigens and neither elicit robust nor anamnestic immune responses (Daniels, 2016), thereby precluding PPVs use in children <2 years of age. In addition, PPVs are poorly immunogenic in the elderly due to immunosenescence (Adler 2017). In contrast, PCVs recruit a T-helper cell immune response and are thereby highly immunogenic and engender a protective, anamnestic immune response in younger and older age groups (Bonten, 2015; Farmaki, 2018; van den Biggelaar, 2019). PCVs have progressively been developed to include new serotypes to increase the breadth of protection against emerging serotypes across the globe.

However, commercialized PCVs only provide protection against the polysaccharide serotypes that comprise the vaccine which presently is at best only 20 of the >100 S. pneumoniae serotypes (Weinberger, 2011; GPSC, 2022). Moreover, PCV implementation is associated with the increased prevalence of non-vaccine S. pneumoniae serotypes in carriage and disease (commonly termed serotype replacement) (Weinberger, 2011; Lee, 2014; Galanis, 2015; Balsells, 2017; Vadlamudi, 2018).

To overcome the serotype limitations of polysaccharide-based vaccines, novel vaccine technologies are being applied to overcome the limitations of traditional chemically conjugated vaccine candidates. The present disclosure provides a serotype-independent, protein-based, pneumococcal vaccine candidate, designed to overcome the serotype limitations of polysaccharide-based vaccines. This approach involves identifying highly conserved S. pneumoniae protein antigens that target virulence factors critical for infection and disease. Several highly conserved pneumococcal proteins with broad serotype coverage have been extensively studied preclinically as vaccine candidates including a genetically detoxified form of the cholesterol-dependent cytolysin, PLY, and CbpA, an associated surface protein involved in bacterial adhesion, invasion of host tissues, and evasion of complement (Mann, 2014; Chen, 2015).

The present disclosure provides an immunogenic fusion protein comprising a genetically detoxified PLY with two conserved peptide fragments of CbpA fused to the toxoid at the N- and C-termini. The immunogenic fusion protein may be adjuvanted with aluminum hydroxide to form a formulation. The inclusion of both PLY and CbpA epitopes in the immunogenic fusion protein is designed to elicit antibodies that will inhibit S. pneumoniae colonization and invasion of host tissues as well as neutralize PLY, the primary cause of tissue damage, inflammation, and disease symptoms, which is advantageous for therapeutic purposes. The serotype-independent approach of the present immunogenic fusion protein both enhances protection provided by PCVs and confers protection well beyond the serotypes that are comprised in commercialized PCV vaccines. Moreover, given that there is minimal/no selective pressure for polysaccharide immune escape, the present immunogenic fusion protein has the capacity to diminish serotype replacement, thereby reducing the need for increased valency PCVs. The use of a single immunogenic fusion protein (e.g. MTRV001) provides significant commercial and manufacturing advantages for use as a vaccine or therapeutic, in comparison to PCVs that are known in the art that require the production of greater than twenty antigens (e.g. PREVNAR 20™).

### Definitions

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this invention is related.

The fusion proteins disclosed herein are immunogenic. As used herein, an “immunogen” is a substance that induces an immune response. The term “immunogenic” refers to the ability of a substance to induce an immune response when administered to an animal. A substance such as a polypeptide displays “increased immunogenicity” relative to another polypeptide when administration of the first polypeptide to an animal results in a greater immune response than that observed with administration of the other polypeptide. An increase in immunogenicity can also refer to not only a greater response in terms of the production of more antibody or T cells but also the production of more protective antibody or T cells. Thus, in specific embodiments, an increase in immunogenicity refers to any statistically significant increase in the level of antibodies or T cells or antibody or T cell production or any statistically significant increase in a protective antibody response. Such an increase can include a 5%, 10%, 20%, 30%, 40%, 50%, 60%, 70%, 80%, 90%, 100% or higher increase in the level of antibodies or in the protective antibody response. The immunogenicity of a polypeptide can be assayed for by measuring the level of antibodies or T cells produced against the polypeptide. Assays to measure for the level of antibodies are known, for example, see Harlow and Lane (1988) Antibodies, A Laboratory Manual(Cold Spring Harbor Publications, New York), for a standard description of antibody generation, immunoassay formats and conditions that can be used to determine specific immunoreactivity. Assays for T cells specific to a polypeptide are known, for example, Rudraraju et al. (2011) Virology 410:429-36, herein incorporated by reference. In other instances, increased immunogenicity can be detected as an improved clinical outcome, as discussed elsewhere herein.

The terms “bind and elute mode”, “bind and elute process”, “bind and elute means”, “binding and elution” as used herein, refer to a separation technique in which at least one immunogenic fusion protein contained in a sample binds to a suitable resin or media (e.g., an affinity chromatography media or a cation exchange chromatography media) and is subsequently eluted.

The terms “flow-through process,” “flow-through mode,” and “flow-through operation,” as used interchangeably herein, refer to a separation technique in which at least one immunogenic fusion protein contained in a biopharmaceutical preparation along with one or more impurities is intended to flow through a material (e.g. flow through anion exchange membrane), which usually binds the one or more impurities, where the immunogenic fusion protein usually does not bind (i.e., flows through)

The term “chromatography” refers to any kind of technique which separates an analyte of interest (e.g. a immunogenic fusion protein) from other molecules present in a mixture through differential adsorption onto a media. Usually, the immunogenic fusion protein is separated from other molecules as a result of differences in rates at which the individual molecules of the mixture migrate through a stationary medium under the influence of a moving phase, or in bind and elute processes.

The term “matrix,” or “means” as used herein, refers to any kind of particulate sorbent, bead, resin or other solid phase (e.g., a membrane, non-woven, monolith, etc.). A matrix having a ligand or functional group attached to it is referred to as “media,” which in a separation process, acts as the adsorbent to separate a target molecule (e.g., an immunogenic fusion protein) from other molecules present in a mixture (e.g., one or more impurities), or alternatively, acts as a sieve to separate molecules based on size (e.g., 0.2 μm filter membrane).

The terms “ion-exchange” and “ion-exchange chromatography,” as used herein, refer to the chromatographic process in which a solute or analyte of interest (e.g., a target molecule being purified) in a mixture, interacts with a charged compound linked (such as by covalent attachment) to a solid phase ion exchange material, such that the solute or analyte of interest interacts non-specifically with the charged compound more or less than solute impurities or contaminants in the mixture. The contaminating solutes in the mixture elute from a column of the ion exchange material faster or slower than the solute of interest or are bound to or excluded from the resin relative to the solute of interest.

The term “ion-exchange chromatography” specifically includes cation exchange, anion exchange, and mixed mode ion exchange chromatography. For example, cation exchange chromatography can bind the target molecule (e.g., an immunogenic fusion protein) followed by elution (e.g., using cation exchange bind and elute chromatography or “CEX”) or can predominately bind the impurities while the target molecule “flows through” the column (cation exchange flow through chromatography FT-CEX). Anion exchange chromatography can bind the target molecule (e.g., an immunogenic fusion peptide of SEQ ID NO: 43) followed by elution or can predominately bind the impurities while the target molecule “flows through” the column, also referred to as negative chromatography. In some embodiments and as demonstrated in the Examples set forth herein, the anion exchange chromatography step is performed in a flow through mode.

The term “ion exchange media” refers to a media that is negatively charged (i.e., a cation exchange media) or positively charged (i.e., an anion exchange media). The charge may be provided by attaching one or more charged ligands to a matrix, e.g., by covalent linkage. Alternatively, or in addition, the charge may be an inherent property of the matrix (e.g., as is the case of silica, which has an overall negative charge).

The term “anion exchange media” is used herein to refer to a media which is positively charged, e.g. having one or more positively charged ligands, such as quaternary amino groups, attached to a matrix. Commercially available anion exchange media include DEAE cellulose, QAE SEPHADEX™ and FAST Q SEPHAROSE™ (GE Healthcare). Other exemplary materials that may be used in the processes and systems described herein are Fractogel® EMID TMAE, Fractogel® EMID TMAE highcap, Eshmuno® Q and Fractogel® EMD DEAE (EMID Millipore).

The term “cation exchange media” refers to a media which is negatively charged, and which has free cations for exchange with cations in an aqueous solution contacted with the solid phase of the media. A negatively charged ligand attached to the solid phase to form the cation exchange media may, for example, be a carboxylate or sulfonate. Commercially available cation exchange media include carboxy-methyl-cellulose, sulphopropyl (SP) immobilized on agarose (e.g., SP-SEPHAROSE FAST FLOW™ or SP-SEPHAROSE HIGH PERFORMANCE™, from GE Healthcare) and sulphonyl immobilized on agarose (e.g. S-SEPHAROSE FAST FLOW™ from GE Healthcare). Preferred is Fractogel® EMD SO₃, Fractogel® EMD SE Highcap, Eshmuno® S and Fractogel® EMID COO (EMID Millipore).

The term “mixed-mode chromatography” or “multi-modal chromatography,” as used herein, refers to a process employing a chromatography stationary phase that carries at least two distinct types of functional groups, each capable of interacting with a molecule of interest. Mixed-mode chromatography generally employs a ligand with more than one mode of interaction with a target protein and/or impurities. The ligand typically includes at least two different but co-operative sites which interact with the substance to be bound. For example, one of these sites may have a charge-charge type interaction with the substance of interest, whereas the other site may have an electron acceptor-donor type interaction and/or hydrophobic and/or hydrophilic interactions with the substance of interest. Electron donor-acceptor interaction types include hydrogen-bonding, π-π, cation-π, charge transfer, dipole-dipole and induced dipole interactions. Generally, based on the differences of the sum of interactions, a target protein and one or more impurities may be separated under a range of conditions.

The term “mixed mode ion exchange media” or “mixed mode media” refers to a media which is covalently modified with cationic and/or anionic and hydrophobic moieties. A commercially available mixed mode ion exchange media is BAKERBOND ABX™ (J. T. Baker, Phillipsburg, N.J.) containing weak cation exchange groups, a low concentration of anion exchange groups, and hydrophobic ligands attached to a silica gel solid phase support matrix. Mixed mode cation exchange materials typically have cation exchange and hydrophobic moieties. Suitable mixed mode cation exchange materials are Hydroxyapatite (HA), Capto® MMC (GE Healthcare) and Eshmuno® HCX (EMD Millipore).

Mixed mode anion exchange materials typically have anion exchange and hydrophobic moieties. Suitable mixed mode anion exchange materials are Capto® Adhere (GE Healthcare).

The term “hydrophobic interaction chromatography” or “HIC,” as used herein, refers to a process for separating molecules based on their hydrophobicity, i.e., their ability to adsorb to hydrophobic surfaces from aqueous solutions. HIC is usually differentiated from the Reverse Phase (RP) chromatography by specially designed HIC resins that typically have a lower hydrophobicity, or density of hydrophobic ligands compared to RP resins. HIC chromatography typically relies on the differences in hydrophobic groups on the surface of solute molecules. These hydrophobic groups tend to bind to hydrophobic groups on the surface of an insoluble matrix. Because HIC employs a more polar, less denaturing environment than reversed phase liquid chromatography, it is becoming increasing popular for protein purification, often in combination with ion exchange or gel filtration chromatography.

The term “impurity” or “contaminant” as used herein, refers to any foreign or objectionable molecule, including a biological macromolecule such as DNA, RNA, one or more host cell proteins, endotoxins, lipids and one or more additives which may be present in a sample containing the target molecule that is being separated from one or more of the foreign or objectionable molecules using a process of the present invention. Additionally, such impurity may include any reagent which is used in a step which may occur prior to the method of the invention. An impurity may be soluble or insoluble in nature.

The term “insoluble impurity,” as used herein, refers to any undesirable or objectionable entity present in a sample containing a target molecule, where the entity is a suspended particle or a solid. Exemplary insoluble impurities include whole cells, cell fragments and cell debris.

The term “soluble impurity,” as used herein, refers to any undesirable or objectionable entity present in a sample containing a target molecule, where the entity is not an insoluble impurity. Exemplary soluble impurities include host cell proteins (HCPs), DNA, RNA, viruses, endotoxins, cell culture media components, lipids etc.

The terms “purifying,” “purification,” “separate,” “separating,” “separation,” “isolate,” “isolating,” or “isolation,” as used herein, refer to increasing the degree of purity of a target molecule from a sample comprising the target molecule and one or more impurities. Typically, the degree of purity of the target molecule is increased by removing (completely or partially) at least one impurity from the sample

A “buffer” is a solution that resists changes in pH by the action of its acid-base conjugate components. Various buffers which can be employed depending, for example, on the desired pH of the buffer, are described in: Buffers. A Guide for the Preparation and Use of Buffers in Biological Systems, Guteffroy, D, ed. Calbiocben Corporation (1975). Non-limiting examples of buffers include MES, MOPS, MOPSO, Tris, HEPES, phosphate, acetate, citrate, succinate, and ammonium buffers, or any combination thereof.

When “loading” a sample onto a device or a column or a separation unit containing a suitable media, a buffer is used to load the sample comprising the target molecule and one or more impurities onto the device or column or separation unit. In the bind and elute mode, the buffer has a conductivity and/or pH such that the target molecule is bound to media, while ideally all the impurities are not bound and flow through the column. Whereas, in a flow-through mode, a buffer is used to load the sample comprising the target molecule and one or more impurities onto a column or device or separation unit, wherein the buffer has a conductivity and/or pH such that the target molecule is not bound to the media and flows through while ideally all or most of the impurities bind to the media.

The term “wash” or “washing” a chromatography media refers to passing an appropriate liquid, e.g., a buffer, through or over the media. Typically washing is used to remove weakly bound contaminants from the media prior to eluting the target molecule and/or to remove non-bound or weakly bound target molecule after loading. In some embodiments, the wash buffer is different from the loading buffer. In other embodiments, the wash buffer and the loading buffer are the same. In a particular embodiment, a wash step is eliminated or the number of wash steps is reduced in a purification process by altering the conditions of the sample load.

The term “elute” or “eluting” or “elution” refers to removal of a molecule (e.g., a polypeptide of interest or an impurity) from a chromatography media by using or altering certain solution conditions, whereby the buffer (referred to as an “elution buffer”) competes with the molecule of interest for the ligand sites on the chromatography resin. A non-limiting example is to elute a molecule from an ion exchange resin by altering the ionic strength of the buffer surrounding the ion exchange material such that the buffer competes with the molecule for the charged sites on the ion exchange material.

### I. Compositions

Compositions disclosed herein provide fusion proteins comprising a first polypeptide operably linked to a second polypeptide. As used herein, “fusion protein” refers to the in frame genetic linkage of at least two heterologous polypeptides. Upon transcription/translation, a single protein is made. In this way, multiple proteins, or fragments thereof can be incorporated into a single polypeptide. “Operably linked” is intended to mean a functional linkage between two or more elements. For example, an operable linkage between two polypeptides fuses both polypeptides together in frame to produce a single polypeptide fusion protein. In particular aspects, the fusion protein further comprises a third polypeptide. Multiple forms of immunogenic fusion proteins are disclosed herein and discussed in detail below.

### A. Fusion Proteins Comprising Immunogenic Regions of Choline Binding Protein A (CbpA)

i. CbpA

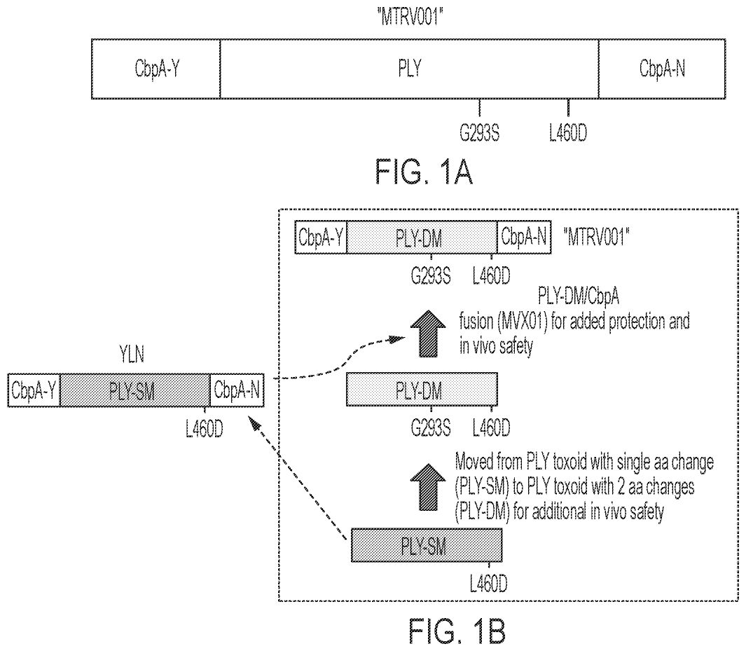

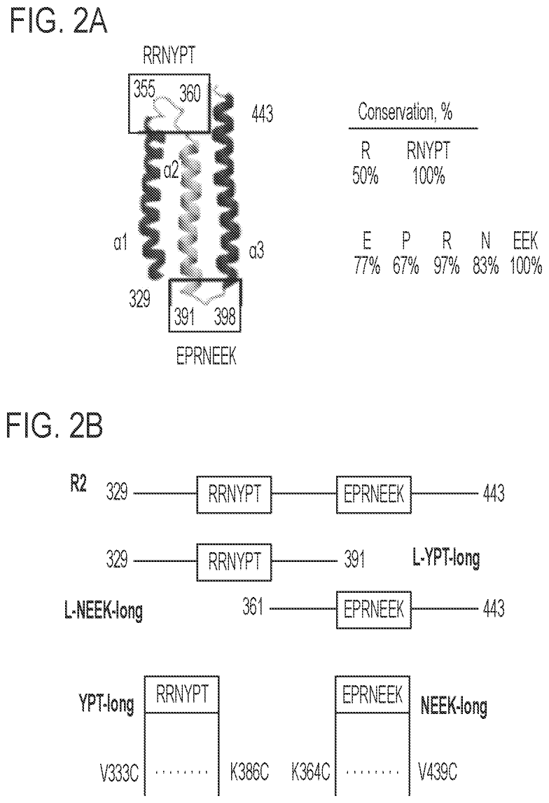

Compositions and methods are provided comprising immunogenic fusion proteins comprising immunogenic regions of the Choline Binding Protein A (CbpA). CbpA is also known as PspC, SpsA, and PbcA. As used herein, a “CbpA fusion protein” can comprise the full CbpA polypeptide or active variants or fragments thereof or any immunogenic fragment of CbpA as discussed in further detail elsewhere herein. CbpA is a 75 kD surface-exposed choline binding protein of Streptococcus pneumoniae. CbpA binds several ligands in the host including pIgR, C3, factor H and laminin receptor. The N-terminus of CbpA (region without the terminal choline binding domain) contains numerous repeats of the leucine zipper motif that cluster within 5 domains termed the A, B, R1, R2, and C domains (FIG. 1). The R2 domain of CbpA (amino acid residues approximately 329 to 443) comprises three anti-parallel alpha-helices (FIG. 2). This three alpha-helix structure is similarly predicted for the R1 domain (Jordan et al. (2006) J. Am. Chem. Soc. 128(28):9119-9128). Notably, the R domains from the TIGR4 strain of S. pneumoniae are highly conserved among CbpA sequences from other pneumococcal strains.

While any immunogenic fragment or domain of CbpA can be used in the fusion proteins disclosed herein, in one embodiment, the fusion protein comprises at least one R2 domain or active variant or fragment of the R2 domain. The R2 domain of CpbA comprises two regions, R2₁ and R2₂, which have been shown to form a loop conformation at each of the two turns of the anti-parallel alpha-helices in the three-dimensional structure of the R2 domain (FIG. 1). As discussed in U.S. Pat. No. 8,722,055 and PCT Application No. PCT/US2012/030241, each of which are herein incorporated by reference in their entirety, the loop conformation of the R2₁ and R2₂ regions increases the immunogenicity of the R2 regions. Thus, the fusion proteins disclosed herein can comprise at least one immunogenic fragment or variant of the R2 domain of CbpA, such as, as least 1, 2, 3, 4, 41 or 42 or more copies of the R2 domain, the R2₁ region and/or the R2₂ region or active variants and fragments thereof.

The R2₁ and R2₂ regions of CbpA have defined functions in disease. The R2₁ region comprises the pIgR binding site. Binding of the R2₁ region of CbpA to the pIgR allows the pneumococcal bacteria to utilize endocytosis machinery to translocate across nasopharyngeal epithelial cells into the blood stream. This binding to pIgR contributes to bacterial colonization of the nasopharynx and invasion of the bacteria into the blood stream.

The R2₁ polypeptide comprises the amino acid sequence set forth in SEQ ID NO: 1 or active variants or fragments thereof. In some embodiments, the immunogenic fusion proteins comprising at least one copy of the R2₁ region or active variants and fragments thereof can produce an immunogenic response which targets bacterial pIgR binding and colonization of the nasopharynx and entry into the blood stream.

The R2₂ region of CbpA comprises the laminin receptor binding site. When the R2 region of CbpA binds to the laminin receptor, it facilitates the hand off of the bacterium to platelet activating factor (PAF) receptor which carries the bacterium into the endothelial cell, across the blood vessel wall, out of the blood stream and into the tissues. Binding to the laminin receptor is a critical step for bacteria to cross the blood brain barrier and cause meningitis. The R2₂ polypeptide comprises the amino acid sequence set forth in SEQ ID NO: 2 or active variants or fragments thereof. In some embodiments, the immunogenic fusion proteins comprising the R2₂ region of CbpA or active variants and fragments thereof can produce an immunogenic response which targets laminin receptor binding, and thus the ability of the bacteria to cross the blood brain barrier and cause meningitis.

In light of the different activities of the R2₁ and R2₂ regions of CbpA, the immunogenic fusion proteins described herein can comprise one or more copies of the R2 regions or an active variant or fragment thereof, one or more copies of either the R2₁ region or the R2₂ region or active variants and fragment thereof, or a combination of both the R2₁ and R2₂ regions or active variant and fragments thereof. In view of the different functional aspects of the R2₁ and R2 regions, one can thereby design a fusion protein having immunogenic activity.

In specific embodiments, the R2₁ and/or R2₂ polypeptide or active variants and fragments thereof employed in the immunogenic fusion protein comprises a loop conformation similar to that present in the native protein. By “loop conformation” is intended a three-dimensional protein structure stabilized in a loop structure by a synthetic linkage in the polypeptide. As used herein, a “synthetic linkage” comprises any covalent or non-covalent interaction that is created in the polypeptides that does not occur in the native protein. Any form of a synthetic linkage that can form a covalent or non-covalent bond between amino acids in the native or variant polypeptides can be used. Such synthetic linkages can include synthetic peptide bonds that are engineered to occur between amino acids present in either the native polypeptide or a variant thereof. The R2₁ and R2₂ polypeptides or active variants and fragments thereof may comprise any form of synthetic linkage that can result in the formation of a covalent bond between amino acids in the native CbpA protein or variant thereof. A synthetic linkage further includes any non-covalent interaction that does not occur in the native polypeptide. For example, loop polypeptides comprising the R2₁ and/or R2₂ region may be engineered to have cysteine residues that are not present in the native CbpA protein and that allow for the formation of a disulfide bridge that stabilizes the polypeptide in a loop conformation. Various methods are known in the art to form such loop conformations in a polypeptide. See, for example, Chhabra et al. (1998) Tetrahedron Lett. 39: 1603-1606; Rohwedder et al. (1998) Tetrahedron Lett. 39: 1175-1178; Wittmann & Seeberger (2000) Angew. Chem. Int. Ed. Engl. 39:4348-4352; and Chan et al. (1995) J. Chem. Soc., Chem. Commun. 21:2209-2210, all of which are herein incorporated by reference in their entirety. Non-limiting examples of R2₁ or R2₂ polypeptides with a loop conformation are discussed in, for example, U.S. Pat. No. 8,722,055 and PCT Application No. PCT/US2012/030241, each of which are herein incorporated by reference in its entirety.

In one embodiment, the loop conformation of the R2₁ and R2₂ polypeptides is generated by at least a first cysteine residue and a second cysteine residue, where the first and the second cysteine residues form a disulfide bond such that the polypeptide is stabilized in a loop conformation. In some specific embodiments, the cysteine residues can be added to the N-terminal and C-terminal ends of the R2₁ and R2₂ polypeptides, or the cysteine residues may be added internally by substituting amino acids within the polypeptide sequence with cysteine residues such that the R2₁ and R2₂ polypeptides form a loop conformation. While not intending to be limited to a particular mechanism, it is believed that stabilization of the R2₁ and R2₂ polypeptides in a loop conformation more closely mimics the native conformation of these polypeptides within the CbpA protein. The R2₁ and R2₂ loop polypeptides thereby have increased protective immunogenicity relative to those polypeptides that are not stabilized in the loop conformation (e.g., linear versions of these polypeptides).

In one non-limiting embodiment, the looped R2₁ and R2₂ polypeptides or active variant or fragments thereof employed in the immunogenic fusion proteins have cysteine substitutions as set forth in SEQ ID NOS: 3 or 4, or active variants or fragments thereof. SEQ ID NO: 3 (AKA YPT) comprises amino acid residues 329-391 of the CbpA protein, wherein the valine at position 333 and the lysine at position 386 have each been substituted with a cysteine residue. SEQ ID NO: 4 (AKA NEEK) comprises amino acid residues 361-443 of the CbpA protein, wherein the lysine at position 364 and the valine at position 439 have each been substituted with a cysteine residue.

Active variants and fragments of the full-length CbpA polypeptide (SEQ ID NO: 12), the CbpA polypeptide without the choline binding domain (R1R2, SEQ ID NO: 13), the R2 domain of the CbpA polypeptide (SEQ ID NO: 14), the R2₁ region (SEQ ID NOS: 1 or 3) and/or the R2₂ region (SEQ ID NOS: 2 or 4) can be employed in the various fusion proteins disclosed herein. Such active variants can comprise at least 65%, 70%, 75%, 80%, 85%, 90%, 91%, 92%, 93%, 94%, 95%, 96%, 97%, 98%, 99% or more sequence identity to SEQ ID NOS: 1, 2, 3, 4, 12, 13, 14, 41 or 42 wherein the active variants retain biological activity and hence are immunogenic. Non-limiting examples of R2₁ and R2₂ polypeptide variants are disclosed, for example, in U.S. Pat. No. 8,722,055 and PCT Application No. PCT/US2012/030241, each of which are herein incorporated by reference. Active fragment can comprises amino acid sequences having at least 5, 10, 15, 20, 25, 30, 35, 40, 50, 60, 70, 80, 100, 150, or more consecutive amino acids of any one of SEQ ID NOS: 1, 2, 3, 4, 12, 13, 14, 41 or 42 where the active fragments retain biological activity and hence are immunogenic.

ii. Other Components of CbpA Fusion Proteins

The immunogenicity of the fusion proteins disclosed herein can be increased through the addition of a heterologous T cell epitope (TCE). Thus, the fusion proteins disclosed herein further comprise at least one heterologous TCE fused in frame to a bacterial polypeptide or variant or fragment thereof (i.e. the CbpA polypeptide or active variant and fragment thereof). Thus, for example, an amino acid sequence for a TCE may be linked to a CbpA polypeptide or active variant or fragment thereof to increase the immunogenicity of the polypeptide relative to that of the same polypeptide lacking the TCE sequence.

As used herein, a “TCE” refers to a polypeptide sequence recognized by T cells. See, for example, El Kasmi et al. (2000) J. Gen. Virol. 81:729-735 and Obeid et al. (1995) J. Virol. 69: 1420-1428; El Kasmi et al. (1999) Vaccine 17:2436-2445; El Kasmi et al. (1998) Mol. Immunol. 35:905-918; El Kasmi et al. (2000) J. Gen. Virol. 81:729-735; Obeid et al. (1995) J. Virol. 69: 1420-1428; and Bouche et al. (2005) Vaccine 23:2074-2077. Polypeptides comprising a TCE sequence are generally between about 10-30, 30-50 or 50-90, or 90-100 amino acids, or up to a full length protein. While any amino acid sequence having a TCE can be used in the in the fusion proteins disclosed herein, non-limiting examples of TCE sequences are set forth in SEQ ID NOS: 15 and 16, or active variants and fragments thereof.

“Heterologous” in reference to a polypeptide is a polypeptide that originates from a different protein. The heterologous TCE sequence can originate from the same organism as the other polypeptide component of the fusion protein, or the TCE can be from a different organism than the other polypeptide components of the fusion protein.

In a specific embodiment, an immunogenic CbpA fusion protein comprises a first polypeptide having an R2₁ or R2₂ region of CbpA, for example, the amino acid sequence of SEQ ID NOS: 1, 2, 3, 4, 41 or 42 or active variants or fragments thereof, wherein the first polypeptide comprising either the R2₁ or R2₂ region of CbpA forms a loop conformation and is immunogenic, and the fusion protein comprises a second polypeptide comprising at least one heterologous TCE, fused in frame to the first polypeptide.

In some embodiments, the heterologous TCE employed in the CbpA fusion protein disclosed herein comprises an immunogenic pneumococcal polypeptide or an active variant or fragment thereof. In such embodiments, in addition to enhancing the immunogenicity of the first polypeptide by providing a TCE, employment of a second immunogenic pneumococcal polypeptide in the CbpA fusion proteins described herein provides another means to target the pneumococcal bacteria and improve immunogenicity against pneumococcal infections. Non-limiting examples of immunogenic pneumococcal proteins which can be employed in the CbpA fusion proteins disclosed herein, include, pneumolysin, pneumococcal surface protein A (PspA), neuraminidase A (nanA), β-N-acetylhexosaminidase (StrH), DnaK, or AliB protein or active variant and fragments thereof. Additional immunogenic pneumococcal polypeptides are known in the art and can be found, for example, in U.S. Pat. Nos. 6,042,838, 6,232,116, U.S. Patent Publication No. 2009/0170162A1, C. C. Daniels et al. (2010) Infection and Immunity 78:2163-72, and Zysk et al. (2000) Infection and Immunity 68:3740-3743, each of which is herein incorporated by reference in their entirety.

In one embodiment, the TCE of the CbpA fusion protein comprises a pneumolysoid polypeptide or a variant or fragment thereof. Pneumolysin is a pore forming toxin and is the major cytolysin produced by Streptococcus pneumoniae. Pneumolysin oligomerizes to form pores in cell membranes and facilitates intrapulmonary bacterial growth and entry into the blood stream by its hemolytic and complement activating properties. The amino acid sequence of wild-type or native pneumolysin is set forth in SEQ ID NO: 5. As used herein, “pneumolysoid” refers to a modified pneumolysin (a pneumolysin toxoid), wherein the modification of the protein inactivates or reduces the oligomerization, hemolytic and/or complement activating properties of the pneumolysoid protein while still retaining immunogenic activity. A reduction in the toxicity of the pneumolysin protein (i.e. a reduction in oligomerization, hemolysis, and/or complement activation) comprises at least a 1%, 5%, 10%, 20%, 30%, 40%, 50%, 60%, 70%, 80%, 90% or greater statistically significant decrease relative to an appropriate control. Various methods to assay for pneumolysin activity are known in the art. Complement activation may be determined, for example, by a two-dimensional gel electrophoresis assay to detect conversion of C3. See, J. C. Paton et al. (1984) Infection and Immunity 43:1085-1087, herein incorporated by reference. Oligomerization of pneumolysin may be assessed, for example, by a combination of sucrose density gradient centrifugation and gel electrophoresis as described in F. D. Saunders et al. (1989) Infection and Immunity 57:2547-2552, herein incorporated by reference. Various pneumolysoids that can be employed in the various immunogenic fusion proteins provided herein are described in, for example, WO2005/108419, WO2005/108580, WO 90/06951, U.S. Patent Application No. 2009/0285846A1 and U.S. Patent Application No. 2010/0166795, which are herein incorporated by reference. WO2005/108419 and WO2005/108580 disclose pneumolysoids having a mutation (e.g. a substitution or deletion) within the region of amino acids 144 to 161 of the wild-type pneumolysin protein. These mutants have reduced oligomerization and/or hemolytic activity as compared to the wild-type pneumolysin, and are therefore less toxic. The mutant may have a substitution or deletion of one or more amino acids 144 to 161 of the wild-type pneumolysin sequence. Thus, the pneumolysoid may have a mutation at one or more of the amino acid residues 144, 145, 146, 147, 148, 149, 150, 151, 152, 153, 154, 155, 156, 157, 158, 159, 160 or 161 of wild-type pneumolysin. In addition, pneumolysoids having reduced hemolytic activity and having at least one amino acid substitution or deletion in at least one of the regions corresponding to amino acids 257-297, 367-397 or 424-437 of the wild-type pneumolysin are described in WO 90/06951.

The pneumolysoid set forth in SEQ ID NO: 7, or an active variant or fragment thereof, comprises a mutation of the lysine at amino acid position 460 to an aspartic acid residue (L460D) which renders the pneumolysoid non-hemolytic. This pneumolysoid is referred to herein as the “L460D” pneumolysoid and is disclosed in U.S. Patent Application No. 2009/0285846A1, herein incorporated by reference in its entirety. An active variant of SEQ ID NO: 7 is provided herein and is set forth in SEQ ID NO: 39. The active variant comprises an amino acid change from Lysine at position 208 to Arginine when compared to SEQ ID NO: 7.

The pneumolysoid set forth in SEQ ID NO: 40, or an active variant or fragment thereof, comprises a mutation of the glycine at amino acid position 293 to a serine residue (G293S) and comprises a mutation of the lysine at amino acid position 460 to an aspartic acid residue (L460D), which renders the pneumolysoid substantially non-toxic (or substantially non-toxic compared to the native PLY protein), substantially non-hemolytic, substantially more stable than the PLY protein, reduces cytolytic activity of the pneumolysoid and/or reduces ability of the pneumolysoid to substantially bind to cell membranes. This pneumolysoid is referred to herein as the “G293S/L460D” or “PLY-DM” pneumolysoid and is disclosed in WO/2016/081839, herein incorporated by reference in its entirety.

The pneumolysoid set forth in SEQ ID NO:8, or an active variant or fragment thereof, comprises a substitution of asparagine in place of aspartic acid at amino acid position 385 and deletion of alanine 146 and arginine 147 of the wild-type pneumolysin sequence (A6N385 pneumolysoid). This A6N385 pneumolysoid is deficient in both hemolysis and complement activation and is disclosed in U.S. Patent Application No. 2010/0166795 and in T. J. Mitchell et al. (1991) Molecular Microbiology 5:1883-1888, herein incorporated by reference in their entirety.

The pneumolysoid set forth in SEQ ID NO: 17, or an active variant or fragment thereof, comprises an amino acid substitution of phenylalanine in place of tryptophan at amino acid position 433 of the wild-type pneumolysin sequence (PdB). This PdB pneumolysoid is deficient in hemolysis and is disclosed in U.S. Pat. No. 6,716,432, herein incorporated by reference in its entirety.

Active variants or fragments of the various pneumolysoids are provided herein. Such active variants can comprise at least 65%, 70%, 75%, 80%, 85%, 90%, 91%, 92%, 93%, 94%, 95%, 96%, 97%, 98%, 99% or more sequence identity to SEQ ID NOS: 40, 5, 7, 8, 17 or 39. An active variant will retain immunogenic activity. Active variants of pneumolysin are well known in the art and find use as pneumolysoids. See, for example, US 2010/0166795 and US 2009/0285846A1 and WO/2016/081839, each of which are herein incorporated by reference in their entirety. The art provides substantial guidance regarding the preparation of such variants, as described elsewhere herein. Thus, in one embodiment, the immunogenic CbpA fusion proteins can comprise the pneumolysoid set forth in SEQ ID NO: 40, 7, 8, 17 or 39 or an active variant thereof having at least 80%, 85%, 90%, 91%, 92%, 93%, 94%, 95%, 96%, 97%, 98%, 99% sequence identity to the amino acid sequence of SEQ ID NO: 40, 7, 8, 17 or 39, wherein the active variant is immunogenic.

iii. Non-limiting Examples of CbpA TCE Fusion Proteins

The immunogenic polypeptides as disclosed herein can be operably linked in a variety of ways to produce an immunogenic fusion protein. When a single CbpA polypeptide or active variant or fragment thereof is employed, the TCE can be fused to the N-terminal end or the C-terminal end of the CbpA polypeptide or active variant or fragment thereof. The fusion protein may comprise other protein components such as a linker peptide between the polypeptides of the fusion protein, or a peptide tag for affinity purification (for example at the N- or C-terminus).

In other embodiments, the CbpA immunogenic fusion proteins can comprise at least 1, 2, 3, 4, 5 or more of the R2₁ or R2₂ regions, or active variants or fragments thereof, operably linked to a heterologous TCE. In one embodiment, the immunogenic fusion protein can comprise a third polypeptide fused in frame to a first polypeptide or a second polypeptide comprising a TCE, wherein the third polypeptide is from a bacteria and is immunogenic. When multiple CbpA polypeptides or variants and fragments thereof are employed in the fusion protein, the TCE can be found at either the N-terminal or C-terminal end of the fusion protein, or alternatively can be located internally in the fusion protein so that it is flanked by CbpA polypeptide sequences. Using multiple regions of the same protein in the fusion protein, in combination with a TCE, may increase immunogenicity to the protein by inducing antibody responses to multiple regions of the protein.

In one embodiment, the immunogenic fusion protein comprises an R2₁ or R2₂ polypeptide in a loop conformation (i.e. SEQ ID NOS: 1, 2, 3, 4, 41 or 42) or active variants or fragments thereof, fused in frame to a heterologous TCE (i.e. a pneumococcal polypeptide or a pneumolysoid polypeptide such as those in SEQ ID NOS: 40, 5, 7, 8, 17 or 39) or active variants or fragments thereof, fused in frame to a second R2₁ or R2₂ polypeptide in a loop conformation (i.e. SEQ ID NOS: 1, 2, 3, 4, 41 or 42) or active variants or fragments thereof. Table 1 provides a non-limiting list of the various structures encompassed by the CbpA fusion proteins disclosed herein.

In a specific embodiment, the immunogenic CbpA fusion protein comprises an R2₁ polypeptide comprising SEQ ID NOS: 1 or 3 or an active variant or fragment thereof in a loop conformation, the L460D pneumolysoid of SEQ ID NO: 7 or 39 or an active variant or fragment thereof, and an R2₂ polypeptide comprising SEQ ID NOS: 2 or 4 or an active variant or fragment thereof in a loop conformation. In a particular embodiment, the immunogenic fusion protein comprises the amino acid sequence set forth in SEQ ID NO: 9 or an active variant or fragment thereof.

In one embodiment, the immunogenic CbpA fusion protein comprises an R2₁ polypeptide comprising SEQ ID NOS: 1 or 3 or an active variant or fragment thereof in a loop conformation, the L460D pneumolysoid of SEQ ID NO: 7 or 39 or an active variant or fragment thereof, and an R2₂ polypeptide comprising SEQ ID NOS: 2 or 4 or an active variant or fragment thereof in a loop conformation. In a particular embodiment, the immunogenic fusion protein comprises the amino acid sequence set forth in SEQ ID NO: 9 or an active variant or fragment thereof.

In other non-limiting embodiments, the immunogenic fusion protein comprises an R2₁ polypeptide comprising SEQ ID NOS: 1 or 3 or an active variant or fragment thereof in a loop conformation, the A6N385 pneumolysoid of SEQ ID NO: 8 or an active variant or fragment thereof, and an R2₂ polypeptide comprising SEQ ID NOS: 2 or 4 or an active variant or fragment thereof in a loop conformation. In a particular embodiment, the immunogenic fusion protein comprises the amino acid sequence set forth in SEQ ID NO: 11 or an active variant or fragment thereof.

In some embodiments, the immunogenic CbpA fusion protein comprises an R2₁ polypeptide comprising SEQ ID NOS: 1, 3 or 41 or an active variant or fragment thereof in a loop conformation, the G293S/L460D pneumolysoid of SEQ ID NO: 40 or an active variant or fragment thereof, and an R22 polypeptide comprising SEQ ID NOS: 2, 4 or 42 or an active variant or fragment thereof in a loop conformation. In a particular embodiment the immunogenic fusion protein comprises the amino acid sequence of SEQ ID NO: 43 or an active variant or fragment thereof.

### Exemplary Immunogenic Fusion Proteins of the Invention

An exemplary CbpA of the invention comprises the amino acid sequence of

(SEQ ID NO: 41)
MACKKAEDQKEEDRRNYPTNTYKTLELECAEGG

A Single underline indicates the Y peptide sequence of choline binding protein A (CbpA).

An exemplary CbpA of the invention comprises the amino acid sequence of

(SEQ ID NO: 42)
KECAKEPRNEEKVKQCK

Double underline indicates the N peptide sequence of CbpA.

A “MTRV001” (also referred to as “CbpA-G293S/L460D pneumolysoid-CbpA” or “CbpA-Y-PLY-DM-CbpA-N” or “CbpA-PLY-DM-CbpA”) immunogenic fusion protein of the invention comprises an amino acid sequence comprising:

(SEQ ID NO: 43)
MACKKAEDQKEEDRRNYPTNTYKTLELECAEGGANKAVNDFILAMNYDKKKLLTHQGESIEN
RFIKEGNQLPDEFVVIERKKRSLSTNTSDISVTATNDSRLYPGALLVVDETLLENNPTLLAV
DRAPMTYSIDLPGLASSDSFLQVEDPSNSSVRGAVNDLLAKWHQDYGQVNNVPARMQYEKIT
AHSMEQLKVKFGSDFEKTGNSLDIDFNSVHSGEKQIQIVNFKQIYYTVSVDAVKNPGDVFQD
TVTVEDLKORGISAERPLVYISSVAYGROVYLKLETTSKSDEVEAAFEALIKGVKVAPQTEW

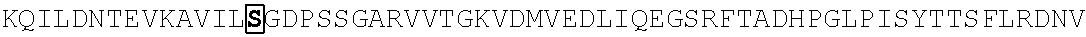

VATFQNSTDYVETKVTAYRNGDLLLDHSGAYVAQYYITWDELSYDHQGKEVLTPKAWDRNGQ

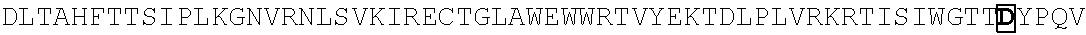

EDKVENDKECAKEPRNEEKVKQCK

A Single underline indicates the Y peptide sequence of choline binding protein A (CbpA). Double underline indicates the N peptide sequence of CbpA. Text with no underline indicate the G293S/L460D pneumolysoid. The 2 boxed letters indicate the amino acid changes (G293S and L460D) of double-mutant pneumolysin genetic toxoid component (PLY-DM) that reduce cytolytic activity. The bolded and underlined font represents sequence critical for binding to human epithelial polymeric immunoglobulin receptor. The bolded and double underlined font represents the sequence critical for binding to the laminin-specific integrin-receptor. The predicted molecular weight of the MTRV001 linear sequences is 58,634 Daltons.

TABLE 1
Examples of CbpA immunogenic fusion proteins

| First Polypeptide | Second Polypeptide | Third Polypeptide |
|---|---|---|
| SEQ ID NOs: 1, 2, 3, 4 or active | TCE | Any bacterial polypeptide |
| variants or fragments thereof | | |
| SEQ ID NOs: 1, 2, 3, 4 or active | Pneumococcal polypeptide or | |
| variants or fragments thereof | active variant or fragment thereof | |
| SEQ ID NOs: 1, 2, 3, 4 or active | Pneumolysoid or active variant or | |
| variants or fragments thereof | fragment thereof | |
| SEQ ID NOs: 1, 2, 3, 4 or active | L460D (SEQ ID NO: 7 or 39 or | |
| variants or fragments thereof | active variant or fragment thereof) | |
| SEQ ID NOs: 1, 2, 3, 4 or active | Δ6N385 (SEQ ID NO: 8 or active | |
| variants or fragments thereof | variant or fragment thereof) | |
| SEQ ID NOs: 1, 2, 3, 4 or active | PdB (SEQ ID NO: 17 or active | |
| variants or fragments thereof | variant or fragment thereof) | |
| SEQ ID NO: 1 or an active variant | Pneumococcal polypeptide or | SEQ ID NO: 1 or an active variant |
| or fragment thereof | active variant or fragment thereof | or fragment thereof |
| SEQ ID NO: 1 or an active variant | Pneumolysoid or active variant or | SEQ ID NO: 1 or an active variant |
| or fragment thereof | fragment thereof | or fragment thereof |
| SEQ ID NO: 1 or an active variant | L460D (SEQ ID NO: 7 or 39 or | SEQ ID NO: 1 or an active variant |
| or fragment thereof | active variant or fragment thereof) | or fragment thereof |
| SEQ ID NO: 1 or an active variant | Δ6N385 (SEQ ID NO: 8 or active | SEQ ID NO: 1 or an active variant |
| or fragment thereof | variant or fragment thereof) | or fragment thereof |
| SEQ ID NO: 1 or an active variant | PdB (SEQ ID NO: 17 or active | SEQ ID NO: 1 or an active variant |
| or fragment thereof | variant or fragment thereof) | or fragment thereof |
| SEQ ID NO: 1 or an active variant | Pneumococcal polypeptide or | SEQ ID NO: 2 or an active variant |
| or fragment thereof | active variant or fragment thereof | or fragment thereof |
| SEQ ID NO: 1 or an active variant | Pneumolysoid or active variant or | SEQ ID NO: 2 or an active variant |
| or fragment thereof | fragment thereof | or fragment thereof |
| SEQ ID NO: 1 or an active variant | L460D (SEQ ID NO: 7 or 39 or | SEQ ID NO: 2 or an active variant |
| or fragment thereof | active variant or fragment thereof) | or fragment thereof |
| SEQ ID NO: 1 or an active variant | Δ6N385 (SEQ ID NO: 8 or active | SEQ ID NO: 2 or an active variant |
| or fragment thereof | variant or fragment thereof) | or fragment thereof |
| SEQ ID NO: 1 or an active variant | PdB (SEQ ID NO: 17 or active | SEQ ID NO: 2 or an active variant |
| or fragment thereof | variant or fragment thereof) | or fragment thereof |
| SEQ ID NO: 1 or an active variant | Pneumococcal polypeptide or | SEQ ID NO: 4 or an active variant |
| or fragment thereof | active variant or fragment thereof | or fragment thereof |
| SEQ ID NO: 1 or an active variant | Pneumolysoid or active variant or | SEQ ID NO: 4 or an active variant |
| or fragment thereof | fragment thereof | or fragment thereof |
| SEQ ID NO: 1 or an active variant | L460D (SEQ ID NO: 7 or 39 or | SEQ ID NO: 4 or an active variant |
| or fragment thereof | active variant or fragment thereof) | or fragment thereof |
| SEQ ID NO: 1 or an active variant | Δ6N385 (SEQ ID NO: 8 or active | SEQ ID NO: 4 or an active variant |
| or fragment thereof | variant or fragment thereof) | or fragment thereof |
| SEQ ID NO: 1 or an active variant | PdB (SEQ ID NO: 17 or active | SEQ ID NO: 4 or an active variant |
| or fragment thereof | variant or fragment thereof) | or fragment thereof |
| SEQ ID NO: 2 or an active variant | Pneumococcal polypeptide or | SEQ ID NO: 1 or an active variant |
| or fragment thereof | active variant or fragment thereof | or fragment thereof |
| SEQ ID NO: 2 or an active variant | Pneumolysoid or active variant or | SEQ ID NO: 1 or an active variant |
| or fragment thereof | fragment thereof | or fragment thereof |
| SEQ ID NO: 2 or an active variant | L460D (SEQ ID NO: 7 or 39 or | SEQ ID NO: 1 or an active variant |
| or fragment thereof | active variant or fragment thereof) | or fragment thereof |
| SEQ ID NO: 2 or an active variant | Δ6N385 (SEQ ID NO: 8 or active | SEQ ID NO: 1 or an active variant |
| or fragment thereof | variant or fragment thereof) | or fragment thereof |
| SEQ ID NO: 2 or an active variant | PdB (SEQ ID NO: 17 or active | SEQ ID NO: 1 or an active variant |
| or fragment thereof | variant or fragment thereof) | or fragment thereof |
| SEQ ID NO: 2 or an active variant | Pneumococcal polypeptide or | SEQ ID NO: 2 or an active variant |
| or fragment thereof | active variant or fragment thereof | or fragment thereof |
| SEQ ID NO: 2 or an active variant | Pneumolysoid or active variant or | SEQ ID NO: 2 or an active variant |
| or fragment thereof | fragment thereof | or fragment thereof |
| SEQ ID NO: 2 or an active variant | L460D (SEQ ID NO: 7 or 39or | SEQ ID NO: 2 or an active variant |
| or fragment thereof | active variant or fragment thereof) | or fragment thereof |
| SEQ ID NO: 2 or an active variant | Δ6N385 (SEQ ID NO: 8 or active | SEQ ID NO: 2 or an active variant |
| or fragment thereof | variant or fragment thereof) | or fragment thereof |
| SEQ ID NO: 2 or an active variant | PdB (SEQ ID NO: 17 or active | SEQ ID NO: 2 or an active variant |
| or fragment thereof | variant or fragment thereof) | or fragment thereof |
| SEQ ID NO: 2 or an active variant | Pneumococcal polypeptide or | SEQ ID NO: 3 or an active variant |
| or fragment thereof | active variant or fragment thereof | or fragment thereof |
| SEQ ID NO: 2 or an active variant | Pneumolysoid or active variant or | SEQ ID NO: 3 or an active variant |
| or fragment thereof | fragment thereof | or fragment thereof |
| SEQ ID NO: 2 or an active variant | L460D (SEQ ID NO: 7 or 39 or | SEQ ID NO: 3 or an active variant |
| or fragment thereof | active variant or fragment thereof) | or fragment thereof |
| SEQ ID NO: 2 or an active variant | Δ6N385 (SEQ ID NO: 8 or active | SEQ ID NO: 3 or an active variant |
| or fragment thereof | variant or fragment thereof) | or fragment thereof |
| SEQ ID NO: 2 or an active variant | PdB (SEQ ID NO: 17 or active | SEQ ID NO: 3 or an active variant |
| or fragment thereof | variant or fragment thereof) | or fragment thereof |
| SEQ ID NO: 3 or an active variant | Pneumococcal polypeptide or | SEQ ID NO: 2 or an active variant |
| or fragment thereof | active variant or fragment thereof | or fragment thereof |
| SEQ ID NO: 3 or an active variant | Pneumolysoid or active variant or | SEQ ID NO: 2 or an active variant |
| or fragment thereof | fragment thereof | or fragment thereof |
| SEQ ID NO: 3 or an active variant | L460D (SEQ ID NO: 7 or 39 or | SEQ ID NO: 2 or an active variant |
| or fragment thereof | active variant or fragment thereof) | or fragment thereof |
| SEQ ID NO: 3 or an active variant | Δ6N385 (SEQ ID NO: 8 or active | SEQ ID NO: 2 or an active variant |
| or fragment thereof | variant or fragment thereof) | or fragment thereof |
| SEQ ID NO: 3 or an active variant | PdB (SEQ ID NO: 17 or active | SEQ ID NO: 2 or an active variant |
| or fragment thereof | variant or fragment thereof) | or fragment thereof |
| SEQ ID NO: 3 or an active variant | Pneumococcal polypeptide or | SEQ ID NO: 3 or an active variant |
| or fragment thereof | active variant or fragment thereof | or fragment thereof |
| SEQ ID NO: 3 or an active variant | Pneumolysoid or active variant or | SEQ ID NO: 3 or an active variant |
| or fragment thereof | fragment thereof | or fragment thereof |
| SEQ ID NO: 3 or an active variant | L460D (SEQ ID NO: 7 or 39 or | SEQ ID NO: 3 or an active variant |
| or fragment thereof | active variant or fragment thereof) | or fragment thereof |
| SEQ ID NO: 3 or an active variant | Δ6N385 (SEQ ID NO: 8 or active | SEQ ID NO: 3 or an active variant |
| or fragment thereof | variant or fragment thereof) | or fragment thereof |
| SEQ ID NO: 3 or an active variant | PdB (SEQ ID NO: 17 or active | SEQ ID NO: 3 or an active variant |
| or fragment thereof | variant or fragment thereof) | or fragment thereof |
| SEQ ID NO: 3 or an active variant | Pneumococcal polypeptide or | SEQ ID NO: 4 or an active variant |
| or fragment thereof | active variant or fragment thereof | or fragment thereof |
| SEQ ID NO: 3 or an active variant | Pneumolysoid or active variant or | SEQ ID NO: 4 or an active variant |
| or fragment thereof | fragment thereof | or fragment thereof |
| SEQ ID NO: 3 or an active variant | L460D (SEQ ID NO: 7 or 39 or | SEQ ID NO: 4 or an active variant |
| or fragment thereof | active variant or fragment thereof) | or fragment thereof |
| SEQ ID NO: 3 or an active variant | Δ6N385 (SEQ ID NO: 8 or active | SEQ ID NO: 4 or an active variant |
| or fragment thereof | variant or fragment thereof) | or fragment thereof |
| SEQ ID NO: 3 or an active variant | PdB (SEQ ID NO: 17 or active | SEQ ID NO: 4 or an active variant |
| or fragment thereof | variant or fragment thereof) | or fragment thereof |
| SEQ ID NO: 4 or an active variant | Pneumococcal polypeptide or | SEQ ID NO: 1 or an active variant |
| or fragment thereof | active variant or fragment thereof | or fragment thereof |
| SEQ ID NO: 4 or an active variant | Pneumolysoid or active variant or | SEQ ID NO: 1 or an active variant |
| or fragment thereof | fragment thereof | or fragment thereof |
| SEQ ID NO: 4 or an active variant | L460D (SEQ ID NO: 7 or 39 or | SEQ ID NO: 1 or an active variant |
| or fragment thereof | active variant or fragment thereof) | or fragment thereof |
| SEQ ID NO: 4 or an active variant | Δ6N385 (SEQ ID NO: 8 or active | SEQ ID NO: 1 or an active variant |
| or fragment thereof | variant or fragment thereof) | or fragment thereof |
| SEQ ID NO: 4 or an active variant | PdB (SEQ ID NO: 17 or active | SEQ ID NO: 1 or an active variant |
| or fragment thereof | variant or fragment thereof) | or fragment thereof |
| SEQ ID NO: 4 or an active variant | Pneumococcal polypeptide or | SEQ ID NO: 3 or an active variant |
| or fragment thereof | active variant or fragment thereof | or fragment thereof |
| SEQ ID NO: 4 or an active variant | Pneumolysoid or active variant or | SEQ ID NO: 3 or an active variant |
| or fragment thereof | fragment thereof | or fragment thereof |
| SEQ ID NO: 4 or an active variant | L460D (SEQ ID NO: 7 or 39 or | SEQ ID NO: 3 or an active variant |
| or fragment thereof | active variant or fragment thereof) | or fragment thereof |
| SEQ ID NO: 4 or an active variant | Δ6N385 (SEQ ID NO: 8 or active | SEQ ID NO: 3 or an active variant |
| or fragment thereof | variant or fragment thereof) | or fragment thereof |
| SEQ ID NO: 4 or an active variant | PdB (SEQ ID NO: 17 or active | SEQ ID NO: 3 or an active variant |
| or fragment thereof | variant or fragment thereof) | or fragment thereof |
| SEQ ID NO: 4 or an active variant | Pneumococcal polypeptide or | SEQ ID NO: 4 or an active variant |
| or fragment thereof | active variant or fragment thereof | or fragment thereof |
| SEQ ID NO: 4 or an active variant | Pneumolysoid or active variant or | SEQ ID NO: 4 or an active variant |
| or fragment thereof | fragment thereof | or fragment thereof |
| SEQ ID NO: 4 or an active variant | L460D (SEQ ID NO: 7 or 39 or | SEQ ID NO: 4 or an active variant |
| or fragment thereof | active variant or fragment thereof) | or fragment thereof |
| SEQ ID NO: 4 or an active variant | Δ6N385 (SEQ ID NO: 8 or active | SEQ ID NO: 4 or an active variant |
| or fragment thereof | variant or fragment thereof) | or fragment thereof |
| SEQ ID NO: 4 or an active variant | PdB (SEQ ID NO: 17 or active | SEQ ID NO: 4 or an active variant |
| or fragment thereof | variant or fragment thereof) | or fragment thereof |
| SEQ ID NO: 41 or an active | G293S/L460D (SEQ ID NO: 40 | SEQ ID NO: 42 or an active |
| variant or fragment thereof | or active variant or fragment | variant or fragment thereof |
| | thereof) | |

*Table 1 denotes a fusion protein with the first polypeptide fused in frame to the second polypeptide optionally fused in frame to the third polypeptide. Reference to active variants and fragments of SEQ ID NOS: 1, 2, 3, 4, 41 or 42 in Table 1 further includes the polypeptide having a loop conformation.

### B. Fusion Proteins Comprising Cytolysoids

As discussed above, the various CbpA fusion proteins provided herein can include a pneumolysoid polypeptide or active variant or fragment thereof to increase immunogenicity against pneumococcal infections. While CbpA is from pneumococcus, it is recognized polypeptides from other type of bacteria could be used to generate an immunogenic fusion protein which can produce protective antibodies against other forms of bacteria, for example, bacteria from the genera Clostridium, Streptococcus, Listeria, Bacillus, and Arcanobacterium.

In one embodiment, the immunogenic fusion protein can comprise a cytolysoid polypeptide or active variant or fragment thereof. As used herein, a “cytolysoid fusion protein” can comprise a full length cytolysoid polypeptide or active variants or fragments thereof or any immunogenic fragment of cytolysoid as discussed in further detail elsewhere herein. Cytolysins are a family of pore-forming toxins that are produced by more than 20 species from the genera Clostridium, Streptococcus, Listeria, Bacillus, and Arcanobacterium. Each cytolysin is produced as a monomer and upon encountering a eukaryotic cell the monomers convert into an oligomeric structure to form a pore complex. Cytolysins are well known as hemolytic proteins. As used herein, “cytolysoid” refers to a modified cytolysin, wherein the modification of the protein inactivates or reduces the oligomerization and/or hemolytic properties of the cytolysoid protein while still retaining immunogenic activity. A reduction in the toxicity of the cytolysin protein (i.e. a reduction in oligomerization, and/or hemolysis) comprises at least a 1%, 5%, 10%, 20%, 30%, 40%, 50%, 60%, 70%, 80%, 90% or greater statistically significant decrease relative to an appropriate control. Various methods to assay for cytolysin activity are known in the art and are the same as described elsewhere herein for pneumolysin.

The art provides substantial guidance regarding the modifications required to inactivate or reduce the toxic activity (i.e. oligomerization and/or hemolysis) of cytolysins. These modifications may be amino acid substitutions, deletions, and/or additions. Such modifications are well known in the art. Some examples include, but are not limited to, WO2005/108419 and WO2005/108580 which disclose cytolysoids having a mutation (e.g. a substitution or deletion) within the region corresponding to amino acids 144 to 161 of the wild-type pneumolysin protein. This region of pneumolysin has a consensus sequence that is shared among the cytolysins. These mutant cytolysins have reduced oligomerization and/or hemolytic activity as compared to the wild-type cytolysin, and are therefore less toxic. The mutant may have a substitution or deletion of one or more amino acids within the regions corresponding to amino acids 144 to 161 of the wild-type pneumolysin sequence. Thus, the cytolysoid may have a mutation at one or more of the amino acids residues corresponding to amino acids 144, 145, 146, 147, 148, 149, 150, 151, 152, 153, 154, 155, 156, 157, 158, 159, 160 or 161 of wild-type pneumolysin. Additional, non-limiting, examples of cytolysoids in the art are disclosed in U.S. Patent Application No. 2009/0285846A1 and U.S. Patent Application No. 2010/0166795, which are herein incorporated by reference.

Any cytolysin can be modified to a cytolysoid and employed in the fusion proteins presented herein. Examples include, but are not limited to, pneumolysin from Streptococcus pneumoniae, perfringolysin O from Clostridium perfringens, intermedilysin from Streptococcus intermedius, alveolysin from Bacillus alvei, anthrolysin from Bacillus anthracis, putative cereolysin from Bacillus cereus, ivanolysin O from Listeria ivanovii, pyolysin from Arcanobacterium pyogenes, seeligeriolysin O from Listeria seeligeri, streptolysin O from S. pyogenes, suilysin from Streptococcus suis, tetanolysin from Clostridium tetani, listeriolysin O from Listeria monocytogenes, streptolysin O from Streptococcus equisimilis, streptolysin O from S. canis, thuringiolysin O from Bacillus thuringiensis, latersporolysin O from B. laterosporus, botulinolysin from Clostridium botulinum, chauveolysin from C. chauvoei, bifermentolysin from C. bifermentans, sordellilysin from C. sordellii, histolyticolysin from Clostridium histiolyticum, novylysin from Clostridium novyi, and septicolysin O from Clostridium septicum. Other examples of cytolysins and cytolysoids can be found, for example in S. E. Gelber et al. (2008) J. Bacteriology 190:3896-3903; and B. H. Jost et al. (2003) Infection and Immunity 71:2966-2969, herein incorporated by reference in their entirety.

The immunogenic cytolysoid fusion proteins provided herein can comprise at least 1, 2, 3, 4, 5 or more immunogenic bacterial polypeptides. The bacterial polypeptide source can include, but is not limited to, the above listed examples of cytolysin comprising bacteria. The immunogenic polypeptides of the cytolysoid fusion proteins disclosed herein can be assembled in various combinations. The cytolysoid can be at either at the N-terminal or C-terminal end of the fusion protein, or it can be flanked by immunogenic bacterial polypeptides. The immunogenic bacterial polypeptides can be from the same bacteria as the cytolysoid or they can be from different bacteria.

In a specific embodiment, the cytolysoid fusion protein comprises a pneumolysoid (i.e. SEQ ID NOS: 40, 7, 8, 17 or 39 or active variants or fragments thereof) and the immunogenic bacterial polypeptides can comprise any immunogenic protein from pneumococcal bacteria.

Active variants or fragments of the various immunogenic cytolysoids are provided herein. Such active variants can comprise at least 65%, 70%, 75%, 80%, 85%, 90%, 91%, 92%, 93%, 94%, 95%, 96%, 97%, 98%, 99% or more sequence identity to a cytolysoid polypeptide provided herein in that they maintain immunogenic activity, as described elsewhere herein. Active variants of immunogenic cytolysoids are known in the art. See, for example, U.S. Patent Application No. 2009/0285846A1 and U.S. Patent Application No. 2010/0166795, herein incorporated by reference in their entirety.

### C. Polynucleotides Encoding the Immunogenic Fusion Proteins and Methods of Making the Immunogenic Fusion Proteins

Compositions further include isolated polynucleotides that encode the various immunogenic fusion proteins described herein above, and variants and fragments thereof. Exemplary polynucleotides comprising nucleotide sequences that encode the various polypeptides and the various fusion proteins are summarized in Table 2. Variants and fragments of the isolated polynucleotides disclosed herein are also encompassed.

TABLE 2
Exemplary polypeptide and nucleic acid sequences of the disclosure

| SEQ ID NO: | AA/NT | Description |
|---|---|---|
| 1 | AA | R2₁ fragment sequence of CbpA of S. pneumoniae |
| 2 | AA | R2₂ fragment sequence of CbpA of S. pneumoniae |
| 3 | AA | Cysteine mutant of R2₁ fragment sequence of S. pneumoniae (AKA “YPT”; |
| | | looped) |
| 4 | AA | Cysteine mutant of R2₂ fragment sequence of S. pneumoniae (AKA “NEEK”; |
| | | looped) |
| 5 | AA | Wild-type pneumolysin amino acid sequence of S. pneumoniae (AKA “PLY”) |
| 6 | NT | Wild-type pneumolysin nucleotide sequence of S. pneumoniae |
| 7 | AA | L460D pneumolysoid sequence |
| 8 | AA | Δ6N385 pneumolysoid sequence |
| 9 | AA | YPT-L460D-NEEK fusion protein sequence |
| 10 | NT | YPT-L460D-NEEK fusion protein nucleotide sequence |
| 11 | AA | YPT-Δ6N385-NEEK fusion protein sequence |
| 12 | AA | Full-length CbpA amino acid sequence of S. pneumoniae |
| 13 | AA | CbpA R1R2 amino acid sequence of S. pneumoniae |
| 14 | AA | R2 domain amino acid sequence of CbpA of S. pneumoniae |
| 15 | AA | TCE1 sequence |
| 16 | AA | TCE2 sequence |
| 17 | AA | PdB pneumolysoid amino acid sequence |
| 18 | AA | PdB-2TCEs-linear NEEK fusion protein sequence |
| 19 | AA | Looped YPT-PdB-2 TCEs-linear NEEK fusion protein sequence |
| 20 | AA | Δ6N385-NEEK fusion protein sequence |
| 21 | AA | Δ6N385-TCE-NEEK fusion protein sequence |
| 22 | AA | ΥPT-Δ6N385 fusion protein sequence |
| 23 | AA | YPT-Δ6N385-TCE-NEEK fusion protein sequence |
| 24 | AA | YPT-L460D-TCE-NEEK fusion protein sequence |
| 25 | AA | L460D-NEEK fusion protein sequence |
| 26 | NT | JAT201 primer |
| 27 | NT | C-term Fusion primer |
| 28 | NT | JAT209 primer |
| 29 | NT | JAT210 primer |
| 30 | NT | Construct 2 primer |
| 31 | NT | PLYNdel primer |
| 32 | NT | NEEKSac1 primer |
| 33 | NT | YPT primer |
| 34 | NT | JAT201b primer |
| 35 | NT | TCENEEK2 primer |
| 36 | NT | JAT215 primer |
| 37 | NT | YPTNDE primer |
| 38 | NT | NeekSac primer |
| 39 | AA | L460D pnemolysoid variant sequence |
| 40 | AA | G293S/L460D pneumolysoid sequence |
| 41 | AA | CbpA variant sequence |
| 42 | AA | CbpA variant sequence |
| 43 | AA | G293S/L460D immunogenic fusion protein |

Vectors and expression cassettes comprising the polynucleotides described herein are further disclosed. Expression cassettes will generally include a promoter operably linked to a polynucleotide and a transcriptional and translational termination region.

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues.

An “isolated” polynucleotide is substantially or essentially free from components that normally accompany or interact with the polynucleotide as found in its naturally occurring environment. Thus, an isolated polynucleotide is substantially free of other cellular material or culture medium when produced by recombinant techniques, or substantially free of chemical precursors or other chemicals when chemically synthesized.

Conventional molecular biology, microbiology, and recombinant DNA techniques within the skill of the art may be employed herein. Such techniques are explained fully in the literature. See, e.g., Sambrook et al., “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

The polypeptides and fusion proteins disclosed herein may be altered in various ways including amino acid substitutions, deletions, truncations, and insertions. Methods for such manipulations are generally known in the art. For example, amino acid sequence variants and fragments of the CbpA or cytolysoid proteins can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. In specific embodiments employing the looped conformation of the R2₁ and R2₂ polypeptides, the mutation comprises at least an insertion or a substitution of a cysteine residue in a CbpA polypeptide disclosed herein. In other embodiments, the mutations in CbpA (the R2 domain, the R2₁ or the R2₂ region) pneumolysin or cytolysins comprise at least a deletion, insertion, and/or amino acid substitution.

A vector which comprises the above-described polynucleotides operably linked to a promoter is also provided herein. A nucleotide sequence is “operably linked” to an expression control sequence (e.g., a promoter) when the expression control sequence controls and regulates the transcription and translation of that sequence. The term “operably linked” when referring to a nucleotide sequence includes having an appropriate start signal (e.g., ATG) in front of the nucleotide sequence to be expressed and maintaining the correct reading frame to permit expression of the sequence under the control of the expression control sequence and production of the desired product encoded by the sequence. If a gene that one desires to insert into a recombinant nucleic acid molecule does not contain an appropriate start signal, such a start signal can be inserted in front of the gene. A “vector” is a replicon, such as plasmid, phage or cosmid, to which another nucleic acid segment may be attached so as to bring about the replication of the attached segment. The promoter may be, or is identical to, a bacterial, yeast, insect or mammalian promoter. Further, the vector may be a plasmid, cosmid, yeast artificial chromosome (YAC), bacteriophage or eukaryotic viral DNA.

Other numerous vector backbones known in the art as useful for expressing protein may be employed. Such vectors include, but are not limited to: adenovirus, simian virus 40 (SV40), cytomegalovirus (CMV), mouse mammary tumor virus (MMTV), Moloney murine leukemia virus, DNA delivery systems, i.e. liposomes, and expression plasmid delivery systems. Further, one class of vectors comprises DNA elements derived from viruses such as bovine papilloma virus, polyoma virus, baculovirus, retroviruses or Semliki Forest virus. Such vectors may be obtained commercially or assembled from the sequences described by methods well-known in the art.

A host vector system for the production of a polypeptide which comprises the vector of a suitable host cell is provided herein. Suitable host cells include, but are not limited to, prokaryotic or eukaryotic cells, e.g. bacterial cells (including gram positive cells), yeast cells, fungal cells, insect cells, and animal cells. Numerous mammalian cells may be used as hosts, including, but not limited to, the mouse fibroblast cell NIH 3T3, CHO cells, HeLa cells, Ltk-cells, etc. Additional animal cells, such as R1.1, B—W and L-M cells, African Green Monkey kidney cells (e.g., COS 1, COS 7, BSC1, BSC40, and BMT10), insect cells (e.g., Sf9), and human cells and plant cells in tissue culture can also be used.

A wide variety of host/expression vector combinations may be employed in expressing the polynucleotide sequences presented herein. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAS, e.g., the numerous derivatives of phage 2, e.g., NM989, and other phage DNA, e.g., M13 and filamentous single stranded phage DNA; yeast plasmids such as the 2p plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences (sequences that control the expression of a nucleotide sequence operably linked to it) may be used in these vectors to express the polynucleotide sequences provided herein. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast α-mating factors, and other sequences known to control the expression of genes of prokaryotic or eukaryotic cells or their viruses, and various combinations thereof.

It will be understood that not all vectors, expression control sequences and hosts will function equally well to express the polynucleotide sequences provided herein. Neither will all hosts function equally well with the same expression system. One skilled in the art will be able to select the proper vectors, expression control sequences, and hosts without undue experimentation to accomplish the desired expression without departing from the scope of this invention. In selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular nucleotide sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the nucleotide sequences to be expressed, and the ease of purification of the expression products.

In preparing the expression cassette, the various polynucleotides may be manipulated, so as to provide for the polynucleotide sequences in the proper orientation and, as appropriate, in the proper reading frame. Toward this end, adapters or linkers may be employed to join the polynucleotides or other manipulations may be involved to provide for convenient restriction sites, removal of superfluous DNA, removal of restriction sites, or the like. For example, linkers such as two glycines may be added between polypeptides. Methionine residues encoded by atg nucleotide sequences may be added to allow initiation of gene transcription. For this purpose, in vitro mutagenesis, primer repair, restriction, annealing, resubstitutions, e.g., transitions and transversions, may be involved.

Further provided is a method of producing a polypeptide which comprises expressing a polynucleotide encoding a fusion protein disclosed herein in a host cell under suitable conditions permitting the production of the polypeptide and recovering the polypeptide so produced.

### D. Variants and Fragments of the Disclosed Polynucleotides and Polypeptides

Active variants and fragments of the disclosed polynucleotides and polypeptides are also employed in the immunogenic fusion proteins described herein. “Variants” refer to substantially similar sequences. As used herein, a “variant polypeptide” is intended to mean a polypeptide derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant polypeptides continue to possess the desired biological activity of the native polypeptide, that is, they are immunogenic. A variant of a polypeptide or polynucleotide sequence disclosed herein (i.e. SEQ ID NOS: 1-25 or 39, 40) will typically have at least about 65%, 70%, 75%, 80%, 85%, 90%, 91%, 92%, 93%, 94%, 95%, 96%, 97%, 98%, 99% or more sequence identity with the reference sequence.

The term “fragment” refers to a portion of an amino acid or nucleotide sequence comprising a specified number of contiguous amino acid or nucleotide residues. In particular embodiments, a fragment of a polypeptide disclosed herein may retain the biological activity of the full-length polypeptide and hence be immunogenic. Fragments of a polynucleotide may encode protein fragments that retain the biological activity of the protein and hence be immunogenic. Alternatively, fragments of a polynucleotide that are useful as PCR primers generally do not retain biological activity. Thus, fragments of a nucleotide sequence disclosed herein (i.e. SEQ ID NOS: 6 or 10) may range from at least about 15, 20, 30, 40, 50, 60, 70, 80, 90, 100, 110, 120, 130, 140, 150, 175, 200, 225, 250, 300, 400, 500, 600, 700, 800, 900, 1000, 1100, 1200, 1300, 1400, or 1500 contiguous nucleotides or up to the full-length polynucleotide. Fragments of a polypeptide sequence disclosed herein (i.e. SEQ ID NOS: 1-5, 7-9, 11, 12-14, 17-25 or 39) may comprise at least 10, 15, 25, 30, 50, 60, 70, 80, 90, 100, 110, 120, 130, 140, 150, 160, 170, 180, 190, 200, 225, 250, 275, 300, 400, 425, 450, 475, or 500 contiguous amino acids, or up to the total number of amino acids present in a full-length protein.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, California, USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence provided herein. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix. By “equivalent program” is intended any sequence comparison program that, for any two sequences in question, generates an alignment having identical nucleotide or amino acid residue matches and an identical percent sequence identity when compared to the corresponding alignment generated by GAP Version 10.

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The above-defined terms are more fully defined by reference to the specification as a whole.

### II. PHARMACEUTICAL COMPOSITIONS AND FORMULATIONS

Compositions further include immunogenic compositions and vaccines comprising an immunogenic fusion protein disclosed herein. Immunogenic compositions provided herein comprise at least one immunogenic fusion protein as described herein in combination with a pharmaceutically acceptable carrier. In some embodiments, the immunogenic fusion protein is present in an amount effective to elicit antibody production when administered to an animal. Methods for detecting antibody production in an animal are well known in the art.

Vaccines for treating or preventing bacterial infection are provided and comprise at least one fusion protein provided herein in combination with a pharmaceutically acceptable carrier, wherein the fusion protein is present in an amount effective for treating or preventing a bacterial infection. In particular embodiments, the vaccine elicits production of protective antibodies against the bacteria when administered to an animal. In specific embodiments, the vaccine comprises an immunogenic fusion protein comprising a cytolysoid. In other embodiments, the vaccine comprises an immunogenic fusion protein comprising a cytolysoid and one or more immunogenic polypeptides from the same bacterial source or a different bacterial source as the cytolysoid.

Vaccines for treating or preventing pneumococcal infection are also provided and comprise at least one fusion protein provided herein in combination with a pharmaceutically acceptable carrier, wherein the fusion protein is present in an amount effective for treating or preventing a pneumococcal infection. In particular embodiments, the vaccine elicits production of protective antibodies against Streptococcus pneumoniae when administered to an animal. In specific embodiments, the vaccine comprises an immunogenic fusion protein comprising a CbpA polypeptide(s) (i.e. such as those fusion proteins presented in Table 1).

In addition, compositions comprising an immunogenic fusion protein or biologically active variant or fragment thereof and an adjuvant in combination with a pharmaceutically acceptable carrier are provided. The immunogenic fusion proteins presented herein can be prepared in an admixture with an adjuvant to prepare a vaccine. Pharmaceutically acceptable carriers and adjuvants are well known in the art. Methods for formulating pharmaceutical compositions and vaccines are generally known in the art. A thorough discussion of formulation and selection of pharmaceutical acceptable carriers, stabilizers, and isomolytes can be found in Remington's Pharmaceutical Sciences (18^{th} ed.; Mack Publishing Company, Eaton, Pennsylvania, 1990), herein incorporated by reference. As provided herein, a vaccine may comprise, for example, at least one of the fusion proteins disclosed in Table 1 or a biologically active variant or fragment thereof.

As described elsewhere herein, the R2₁ region of CbpA is believed to be involved in bacterial colonization of the nasopharynx while both the R2₁ and R2₂ region of CbpA mediates bacterial invasion of host cells. Thus, a vaccine that comprises a fusion protein comprising both an R2₁ and an R2₂ polypeptide can provide protection against both steps involved in pneumococcal infection. In specific embodiments, a vaccine comprising a fusion protein comprising both an R2₁ and an R2₂ polypeptide, for example, the fusion protein of SEQ ID NO: 43 or active variants or fragments thereof, may provide protection against both steps involved in pneumococcal infection.

The immunogenic compositions and vaccines disclosed herein may comprise a mixture of 1 or more fusion proteins with 1 or more polypeptides provided herein. A vaccine may comprise, for example, any one of the immunogenic fusion proteins described in Table 1 or active variants or fragments thereof combined as a mixture with one or more of the polypeptides of SEQ ID NOS: 1, 2, 3, 4, 5, 7, 8, 12, 13, 14, 17, 39, 40, 41, 42, 43 or active variants or fragments thereof. In some embodiments, the vaccine comprises SEQ ID NO: 43.

The immunogenic compositions may be formulated in liquid form (i.e. solutions or suspensions) or in a lyophilized form. Liquid formulations may advantageously be administered directly from their packaged form and are thus ideal for injection without the need for reconstitution in aqueous medium as otherwise required for lyophilized compositions of the invention.

Formulation of the immunogenic composition of the present disclosure can be accomplished using art-recognized methods. For instance, the individual polysaccharides and/or conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions. The present disclosure provides a formulation comprising any of combination of the immunogenic compositions disclosed herein and a pharmaceutically acceptable excipient, carrier, or diluent.

In another embodiment, the immunogenic compositions of the present invention are administered orally and are thus, formulated in a form suitable for oral administration, i.e., as a solid or a liquid preparation. Solid oral formulations include tablets, capsules, pills, granules, pellets and the like. Liquid oral formulations include solutions, suspensions, dispersions, emulsions, oils and the like.

Pharmaceutically acceptable carriers for liquid formulations are aqueous or non-aqueous solutions, suspensions, emulsions or oils. Examples of nonaqueous solvents are propylene glycol, polyethylene glycol, and injectable organic esters such as ethyl oleate. In an embodiment, the immunogenic composition of the disclosure is in liquid form, preferably in aqueous liquid form.

Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

In one embodiment, the present disclosure provides a container filled with any of the immunogenic compositions disclosed herein. In one embodiment, the container is selected from the group consisting of a vial, a syringe, a flask, a fermentor, a bioreactor, a bag, ajar, an ampoule, a cartridge and a disposable pen. In certain embodiments, the container is siliconized.

In an embodiment, the container of the present disclosure is made of glass, metals (e.g., steel, stainless steel, aluminum, etc.) and/or polymers (e.g., thermoplastics, elastomers, thermoplastic-elastomers). In an embodiment, the container of the present disclosure is made of glass.

In one embodiment, the present disclosure provides a syringe filled with any of the immunogenic compositions disclosed herein. In certain embodiments, the syringe is siliconized and/or is made of glass. The immunogenic compositions of the invention can be formulated as single dose vials, multi-dose vials or as pre-filled glass or plastic syringes.

The immunogenic compositions of the instant invention may be isotonic, hypotonic or hypertonic. However, it is often preferred that a composition for infusion or injection be essentially isotonic, when administrated. Hence, for storage, a composition may preferably be isotonic or hypertonic. If the composition is hypertonic for storage, it may be diluted to become an isotonic solution prior to administration.

The isotonic agent may be an ionic isotonic agent such as a salt or a non-ionic isotonic agent such as a carbohydrate. Examples of ionic isotonic agents include but are not limited to NaCl, CaCh, KCl and MgCh. Examples of non-ionic isotonic agents include but are not limited to mannitol, sorbitol and glycerol.

It is also preferred that at least one pharmaceutically acceptable additive is a buffer. For some purposes, for example, when the pharmaceutical composition is meant for infusion or injection, it is often desirable that the composition comprises a buffer, which is capable of buffering a solution to a pH in the range of 4 to 10, such as 5 to 9, for example 6 to 8.

In some embodiments, the composition or the formulation of the disclosure has a pH level between pH of 6 to pH 9. In some embodiments, the composition or the formulation of the disclosure has a pH level between pH of 5.5 to pH 7.5. In some embodiments, the composition or the formulation has a pH of about 7.4.

The compositions or the formulations of the disclosure may comprise at least one buffer. The buffer may be selected from USP compatible buffers for parenteral use, in particular, when the pharmaceutical formulation is for parenteral use. For example, the buffer may be selected from the group consisting of monobasic acids such as acetic, benzoic, gluconic, glyceric and lactic; dibasic acids such as aconitic, adipic, ascorbic, carbonic, glutamic, malic, succinic and tartaric, polybasic acids such as citric and phosphoric; and bases such as ammonia, diethanolamine, glycine, triethanolamine, and TRIS. Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, glycols such as propylene glycols or polyethylene glycol, Polysorbate 80 (PS-80), Polysorbate 20 (PS-20), and Poloxamer 188 (P188) are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs. The buffer may, for example, be selected from the group consisting of TRIS, acetate, glutamate, lactate, maleate, tartrate, phosphate, citrate, carbonate, glycinate, histidine, glycine, succinate, HEPES (4-(2-hydroxyethyl)-1-piperazineethanesulfonic acid), MOPS (3-(N-morpholino)propanesulfonic acid), MES (2-(/V-morpholino)ethanesulfonic acid) and triethanolamine buffer.

In some embodiments, the concentration of buffer will range from about 1 mM to about 100 mM. In some embodiments, the concentration of buffer will range from about 10 mM to about 80 mM. In some embodiments, the concentration of buffer will range from about 1 mM to about 50 mM, or about 5 mM to about 50 mM.

In some embodiments, the buffer is a phosphate buffer. In some embodiments, the buffer is a sodium phosphate buffer. In some embodiments, the composition or the formulation comprises a sodium phosphate buffer. In some embodiments, the concentration of the sodium phosphate buffer is between about 1 mM to about 50 mM. In some embodiments, the concentration of the sodium phosphate buffer is between about 5 mM to about 50 mM, about 5 mM to about 45 mM, about 5 mM to about 40 mM, about 5 mM to about 35 mM, about 5 mM to about 30 mM, about 5 mM to about 25 mM, about 5 mM to about 20 mM, about 5 mM to about 15 mM, about 5 mM to about 10 mM, about 10 mM to about 50 mM, 10 mM to about 45 mM, about 10 mM to about 40 mM, about 10 mM to about 35 mM, about 10 mM to about 30 mM, about 10 mM to about 25 mM, about 10 mM to about 20 mM, about 10 mM to about 15 mM, about 15 mM to about 50 mM, about 15 mM to about 45 mM, about 15 mM to about 40 mM, about 15 mM to about 35 mM, about 15 mM to about 30 mM, about 15 mM to about 25 mM, about 15 mM to about 20 mM, about 20 mM to about 45 mM, about 20 mM to about 40 mM, about 20 mM to about 35 mM, about 20 mM to about 30 mM, about 20 mM to about 25 mM, about 25 mM to about 45 mM, about 25 mM to about 40 mM, about 25 mM to about 35 mM, about 25 mM to about 30 mM, about 30 mM to about 45 mM, about 30 mM to about 40 mM, about 30 mM to about 35 mM, about 35 mM to about 45 mM, about 35 mM to about 40 mM, or about 40 mM to about 45 mM. In some embodiments, the final concentration of the sodium phosphate buffer is at a final concentration of about 5 mM, about 6 mM, about 7 mM, about 8 mM, about 9 mM, about 10 mM, about 11 mM, about 12 mM, about 13 mM, about 14 mM, or about 15 mM. In some embodiments, the composition or the formulation comprises a sodium phosphate buffer at a concentration of about 10 mM. In some embodiments, the final concentration of the sodium phosphate buffer is about 9 mM.

In an embodiment, the composition or the formulation of the disclosure comprises a salt. In some embodiments, the salt is selected from the groups consisting of magnesium chloride, potassium chloride, calcium chloride, sodium chloride and a combination thereof. In one particular embodiment, the salt is sodium chloride. Non-ionic isotonic agents including but not limited to sucrose, trehalose, mannitol, sorbitol and glycerol may be used in lieu of a salt. Suitable salt ranges include, but are not limited to, 20 mM to 500 mM or 40 mM to 170 mM. In one embodiment, the immunogenic compositions of the invention comprise sodium chloride. In certain embodiments, the sodium chloride is at a final concentration of about 100 mM to about 500 mM, about 100 mM to about 400 mM, about 100 mM to about 300 mM or of about 100 mM to about 200 mM. In certain embodiments, the buffer is sodium chloride at a final concentration of about 125 mM to about 175 mM. In certain embodiments, the final concentration of the sodium chloride is about 130 mM to about 160 mM. In certain embodiments, the final concentration of the sodium chloride is about 135 mM, about 136 mM, about 137 mM, about 138 mM, about 139 mM, about 140 mM, about 141 mM, about 142 mM, about 143 mM, about 144 mM, about 145 mM, about 146 mM about 147 mM, about 148 mM, about 149 mM, about 150 mM, about 151 mM, about 152 mM, about 153 mM, about 154 mM or about 155 mM. In some embodiments, the final concentration of the sodium chloride is about 154 mM. In some embodiments, the final concentration of the sodium chloride is about 138.6 mM. In some embodiments, the final concentration of the sodium chloride is about 139 mM.

In an embodiment, the immunogenic compositions of the disclosure comprise a surfactant. Surfactants may include, but are not limited to polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxy stearate (PEG-15, Solutol H 15), polyoxyethylene-35-ricinoleate (CREMOPHOR® EL), soy lecithin, a poloxamer, Poloxamer-188 (P188; Pluoronic; F68 NF), copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypoly ethoxy ethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Mixtures of surfactants can be used.

Preferred amounts of surfactants (% by weight) are: polyoxyethylene sorbitan esters (such as PS-80) of from 0.01 to 1%, in particular about 0.01%; octyl- or nonylphenoxy polyoxyethanols (such as Triton X-100, or other detergents in the Triton series) of from 0.001 to 0.1%, in particular 0.005 to 0.02%; polyoxyethylene ethers (such as laureth 9) of from 0.1 to 20%, preferably 0.1 to 10% and in particular 0.1 to 1% or about 0.5%.

In some embodiments, the composition or the formulation of the invention comprises a surfactant. In one embodiment, the surfactant is polysorbate 20 (Tween 20). In some embodiments, the Tween 20 is at a final concentration of about 1 μg/mL to about 600 μg/mL. In certain embodiments, the Tween 20 is at a final concentration of about 100 μg/mL to about 200 μg/mL, about 200 μg/mL to about 300 μg/mL, about 300 μg/mL to about 400 μg/mL, about 400 μg/mL to about 500 μg/mL or about 500 μg/mL to about 600 μg/mL.

In some embodiments, the Tween 20 is at a final concentration of about 100 μg/mL to about 500 μg/mL, about 100 μg/mL to about 475 μg/mL, about 100 μg/mL to about 450 μg/mL, about 100 μg/mL to about 425 μg/mL, about 100 μg/mL to about 400 μg/mL, about 100 μg/mL to about 375 μg/mL, about 100 μg/mL to about 350 μg/mL, about 100 μg/mL to about 325 μg/mL, about 100 μg/mL to about 300 μg/mL, about 100 μg/mL to about 275 μg/mL, about 100 μg/mL to about 250 μg/mL, about 100 μg/mL to about 225 μg/mL, about 100 μg/mL to about 200 μg/mL, about 100 μg/mL to about 175 μg/mL, about 100 μg/mL to about 150 μg/mL, about 100 μg/mL to about 125 μg/mL, about 125 μg/mL to about 500 μg/mL, about 125 μg/mL to about 475 μg/mL, about 125 μg/mL to about 450 μg/mL, about 125 μg/mL to about 425 μg/mL, about 125 μg/mL to about 400 μg/mL, about 125 μg/mL to about 375 μg/mL, about 125 μg/mL to about 350 μg/mL, about 125 μg/mL to about 325 μg/mL, about 125 μg/mL to about 300 μg/mL, about 125 μg/mL to about 275 μg/mL, about 125 μg/mL to about 250 μg/mL, about 125 μg/mL to about 225 μg/mL, about 125 μg/mL to about 200 μg/mL, about 125 μg/mL to about 175 μg/mL, about 125 μg/mL to about 150 μg/mL, about 175 μg/mL to about 500 μg/mL, about 175 μg/mL to about 475 μg/mL, about 175 μg/mL to about 450 μg/mL, about 175 μg/mL to about 425 μg/mL, about 175 μg/mL to about 400 μg/mL, about 175 μg/mL to about 375 μg/mL, about 175 μg/mL to about 350 μg/mL, about 175 μg/mL to about 325 μg/mL, about 175 μg/mL to about 300 μg/mL, about 175 μg/mL to about 275 μg/mL, about 175 μg/mL to about 250 μg/mL, about 175 μg/mL to about 225 μg/mL, about 175 μg/mL to about 200 μg/mL, about 200 μg/mL to about 500 μg/mL, about 200 μg/mL to about 475 μg/mL, about 200 μg/mL to about 450 μg/mL, about 200 μg/mL to about 425 μg/mL, about 200 μg/mL to about 400 μg/mL, about 200 μg/mL to about 375 μg/mL, about 200 μg/mL to about 350 μg/mL, about 200 μg/mL to about 325 μg/mL, about 200 μg/mL to about 300 μg/mL, about 200 μg/mL to about 275 μg/mL, about 200 μg/mL to about 250 μg/mL, about 200 μg/mL to about 225 μg/mL, about 225 μg/mL to about 500 μg/mL, about 225 μg/mL to about 475 μg/mL, about 225 μg/mL to about 450 μg/mL, about 225 μg/mL to about 425 μg/mL, about 225 μg/mL to about 400 μg/mL, about 225 μg/mL to about 375 μg/mL, about 225 μg/mL to about 350 μg/mL, about 225 μg/mL to about 325 μg/mL, about 225 μg/mL to about 300 μg/mL, about 225 μg/mL to about 275 μg/mL, about 225 μg/mL to about 250 μg/mL, about 250 μg/mL to about 500 μg/mL, about 250 μg/mL to about 475 μg/mL, about 250 μg/mL to about 450 μg/mL, about 250 μg/mL to about 425 μg/mL, about 250 μg/mL to about 400 μg/mL, about 250 μg/mL to about 375 μg/mL, about 250 μg/mL to about 350 μg/mL, about 250 μg/mL to about 325 μg/mL, about 250 μg/mL to about 300 μg/mL, about 250 μg/mL to about 275 μg/mL, about 275 μg/mL to about 500 μg/mL, about 275 μg/mL to about 475 μg/mL, about 275 μg/mL to about 450 μg/mL, about 275 μg/mL to about 425 μg/mL, about 275 μg/mL to about 400 μg/mL, about 275 μg/mL to about 375 μg/mL, about 275 μg/mL to about 350 μg/mL, about 275 μg/mL to about 325 μg/mL, about 275 μg/mL to about 300 μg/mL, about 300 μg/mL to about 500 μg/mL, about 300 μg/mL to about 475 μg/mL, about 300 μg/mL to about 450 μg/mL, about 300 μg/mL to about 425 μg/mL, about 300 μg/mL to about 400 μg/mL, about 300 μg/mL to about 375 μg/mL, about 300 μg/mL to about 350 μg/mL, about 300 μg/mL to about 325 μg/mL, about 325 μg/mL to about 500 μg/mL, about 325 μg/mL to about 475 μg/mL, about 325 μg/mL to about 450 μg/mL, about 325 μg/mL to about 425 μg/mL, about 325 μg/mL to about 400 μg/mL, about 325 μg/mL to about 375 μg/mL, about 325 μg/mL to about 350 μg/mL, about 350 μg/mL to about 500 μg/mL, about 350 μg/mL to about 475 μg/mL, about 350 μg/mL to about 450 μg/mL, about 350 μg/mL to about 425 μg/mL, about 350 μg/mL to about 400 μg/mL, about 350 μg/mL to about 375 μg/mL, about 375 μg/mL to about 500 μg/mL, about 375 μg/mL to about 475 μg/mL, about 375 μg/mL to about 450 μg/mL, about 375 μg/mL to about 425 μg/mL, about 375 μg/mL to about 400 μg/mL, about 400 μg/mL to about 500 μg/mL, about 400 μg/mL to about 475 μg/mL, about 400 μg/mL to about 450 μg/mL, about 400 μg/mL to about 425 μg/mL, about 425 μg/mL to about 500 μg/mL, about 425 μg/mL to about 475 μg/mL, about 425 μg/mL to about 450 μg/mL, about 450 μg/mL to about 500 μg/mL, about 450 μg/mL to about 475 μg/mL or 475 μg/mL to about 500 μg/mL. In some embodiments, the Tween 20 is at a final concentration of about 275 μg/mL.

In another embodiment, the Tween 20 is at a final concentration of about 1 μg/mL to about 100 μg/mL. In some embodiments, the Tween 20 is at a final concentration of about 4 μg/mL to about 8 μg/mL. In some embodiments, the Tween 20 is at a final concentration of about 12 μg/mL to about 24 μg/mL. In some embodiments, the Tween 20 is at a final concentration of about 23 μg/mL to about 38 μg/mL. In some embodiments, the Tween20 is at about 35 μg/mL to about 73 μg/mL. In some embodiments, the Tween 20 is at a final concentration of about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80 μg/mL.

In some embodiments, the immunogenic compositions disclosed herein may further comprise at least one, two or three adjuvants. In some embodiments, the immunogenic compositions disclosed herein may further comprise one adjuvant. The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Exemplary adjuvants to enhance effectiveness of the immunogenic compositions as disclosed herein include, but are not limited to: (1) oil-in-water emulsion formulations (with or without other specific immunostimulating agents such as muramyl peptides (see below) or bacterial cell wall components), such as for example (a) SAF, containing 10% Squalane, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, MT) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, MA), ABISCO® (Isconova, Sweden), or ISCOMATRIX® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+IM2 (optionally+a sterol) (e.g., WO 98/57659); (13) other substances that act as immunostimulating agents to enhance the efficacy of the composition. Muramyl peptides include but are not limited to N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE.

In an embodiment, the composition or the formulation disclosed herein comprises aluminum salts (alum) as the adjuvant. In some embodiments the composition or the formulation comprises aluminum phosphate, aluminum sulfate or aluminum hydroxide. In some embodiments, the final concentration of the adjuvant is between about 0.01 mg/mL to about 3.0 mg/mL. In some embodiments, the final concentration of the adjuvant is between about 0.01 mg/mL to about 1.0 mg/mL. In some embodiments, the final concentration of the adjuvant is between about 0.5 mg/mL to about 2 mg/mL.

In some embodiments, the composition or the formulation disclosed herein comprises aluminum phosphate or aluminum hydroxide as adjuvant. In some embodiments the adjuvant is aluminum hydroxide. In some embodiments, the aluminum hydroxide is Alhydrogel®.

In one embodiment, the adjuvant is at a final concentration of about 0.1 mg/mL to about 2.0 mg/mL. In some embodiments, the adjuvant is at a final concentration of about 0.5 mg/mL to about 1.5 mg/mL, about 0.6 mg/mL to about 1.4 mg/mL, about 0.7 mg/mL to about 1.3 mg/mL, about 0.8 mg/mL to about 1.2 mg/mL or about 0.9 mg/mL to about 1.1 mg/mL. In some embodiments, the adjuvant is at a final concentration of about 1 mg/mL.

The composition or the formulation of the disclosure comprises an immunogenic fusion protein (e.g., SEQ ID NO: 43). In some embodiments, the immunogenic fusion protein is at a final concentration of about 1 μg/mL to about 100 μg/mL, about 1 μg/mL to about 200 μg/mL, about 1 μg/mL to about 300 μg/mL, about 1 μg/mL to about 400 μg/mL or about 1 μg/mL to about 500 μg/mL. In some embodiments, the immunogenic fusion protein is at a concentration of about 10 to about 30 μg/mL, about 48 μg/mL to about 72 μg/mL, about 96 μg/mL to about 124 μg/mL or about 144 μg/mL to about 216 μg/mL. In some embodiments, the immunogenic fusion protein is at a concentration of about 10 μg/mL, about 15 μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, about 100 μg/mL, about 105 μg/mL, about 110 μg/mL, about 115 μg/mL, about 120 μg/mL, about 125 μg/mL, about 130 μg/mL, about 135 μg/mL, about 140 μg/mL, about 145 μg/mL, about 150 μg/mL, about 155 μg/mL, about 160 μg/mL, about 165 μg/mL, about 170 μg/mL, about 175 μg/mL, about 180 μg/mL, about 185 μg/mL, about 190 μg/mL, about 195 μg/mL, about 200 μg/mL, about 205 μg/mL, about 210 μg/mL, about 215 μg/mL, about 220 μg/mL, about 225 μg/mL, about 230 μg/mL, about 235 μg/mL, about 240 μg/mL, about 245 μg/mL, about 250 μg/mL, about 255 μg/mL, about 260 μg/mL, about 265 μg/mL, about 270 μg/mL, about 275 μg/mL, about 280 μg/mL, about 285 μg/mL, about 290 μg/mL or about 300 μg/mL or any concentration in between. In some embodiments, the immunogenic fusion protein is at a final concentration of about 20 μg/mL. In some embodiments, the immunogenic fusion protein is at a final concentration of about 60 μg/mL. In some embodiments, the immunogenic fusion protein is at a final concentration of about 120 μg/mL. In some embodiments, the immunogenic fusion protein is at a final concentration of about 180 μg/mL.

The amount of immunogenic fusion protein in each dose of the composition or the formulation is selected as an amount that induces an immuno-protective response without significant, adverse effects. In some embodiments of the invention, the dose of the immunogenic fusion protein is from about 1 μg to about 150 μg, about 1 μg to about 200 μg, about 1 μg to about 250 μg, about 1 μg to about 300 μg, about 1 μg to about 350 μg, about 1 μg to about 400 μg, about 1 μg to about 450 μg or about 1 μg to about 500 μg. In some embodiments, the dose of the immunogenic fusion protein is from about 5 μg to about 15 μg, about 48 μg to about 72 μg, about 96 μg to about 116 μg, about 144 μg to about 216 μg. In some embodiments, the dose of the immunogenic fusion protein is about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 105 μg, about 110 μg, about 115 μg, about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, about 200 μg, about 205 μg, about 210 μg, about 215 μg, about 220 μg, about 225 μg, about 230 μg, about 235 μg, about 240 μg, about 245 μg, about 250 μg, about 255 μg, about 260 μg, about 265 μg, about 270 μg, about 275 μg, about 280 μg, about 285 μg, about 290 μg or about 300 μg or any amount in between. In some embodiments, the dose of the immunogenic fusion protein is about 10 μg. In some embodiments, the dose of the immunogenic fusion protein is about 30 μg. In some embodiments, the dose of the immunogenic fusion protein is about 60 μg. In some embodiments, the dose of the immunogenic fusion protein is about 90 μg.

The disclosure provides an injectable formulation comprising an immunogenic fusion protein of SEQ ID NO: 43 at a final concentration of about 0.5 mg/mL to about 1.5 mg/mL, sodium phosphate buffer at final concentration of about 10 mM, NaCl at a final concentration of about 154 mM, and polysorbate 20 (Tween20) at a final concentration of about 275 μg/mL, wherein the pH of the injectable formulation is about 7.4.

The disclosure provides an injectable formulation comprising an immunogenic fusion protein of SEQ ID NO: 43 at a final concentration of about 0.5 mg/mL to about 1.5 mg/mL, sodium phosphate buffer at final concentration of about 10 mM, NaCl at a final concentration of about 154 mM, and Tween 20 at a final concentration of about 275 μg/mL, wherein the pH of the injectable formulation is about 7.4.

Exemplary formulations of the immunogenic compositions of the invention are shown in Table 3 and Table 4.

TABLE 3
Drug Substance Formulation

| Formulation | Amounts | |
|---|---|---|
| Immunogenic fusion protein | 1.0 ± 0.2 | mg/mL |
| Sodium phosphate buffer | 10 | mM |
| NaCl | 154 | mM |
| Tween 20 | 275 | μg/mL |

| | |
|---|---|
| pH | 7.4 |

TABLE 4
Drug Product Formulation

| | Formulation | Formulation | Formulation | Formulation |
|---|---|---|---|---|
| | 1 | 2 | 3 | 4 |
| Dose Level of | 10 μg | 30 μg | 60 μg | 90 μg |
| immunogenic fusion | | | | |
| protein^{a}: | | | | |
| Immunogenic fusion | 10-30 | 48-72 | 96-124 | 144-216 |
| protein (μg/mL) | | | | |
| Phosphate buffer (mM) | 9 | 9 | 9 | 9 |
| NaCl (mM) | 139 | 139 | 139 | 139 |
| Tween 20 (μg/mL) | 4-8 | 12-24 | 23-38 | 35-73 |
| Alhydrogel (mg/mL) | 1.0 ± 0.1 | 1.0 ± 0.1 | 1.0 ± 0.1 | 1.0 ± 0.1 |
| Fill Volume (mL) | 1.0 ± 0.1 | 1.0 ± 0.1 | 1.0 ± 0.1 | 1.0 ± 0.1 |
| pH | 7.4 | 7.4 | 7.4 | 7.4 |

^{a}Dose is 0.5 mL of the relevant strength.

In some embodiments, the drug product formulation comprises 180 μg/mL of the immunogenic fusion protein, 1 mg/mL aluminum (in the form of aluminum hydroxide) in 9 mM sodium phosphate, 139 mM sodium chloride, 275 μg/mL polysorbate 20, pH 7.4.

The disclosure also provides and injectable formulation in a multiple unit dose vial containing about 1 mL wherein the injectable formulation comprises an immunogenic fusion protein of SEQ ID NO: 43 at a final concentration of about 20 μg/mL, sodium phosphate buffer at final concentration of about 9 mM, NaCl at a final concentration of about 139 mM, polysorbate 20 at a final concentration of about 275 μg/mL, and aluminum hydroxide at a final concentration of about 1 mg/mL, wherein the pH of the injectable formulation is about 7.4.

The disclosure also provides and injectable formulation in a multiple unit dose vial containing about 1 mL wherein the injectable formulation comprises an immunogenic fusion protein of SEQ ID NO: 43 at a final concentration of about 60 μg/mL, sodium phosphate buffer at final concentration of about 9 mM, NaCl at a final concentration of about 139 mM, polysorbate 20 at a final concentration of about 275 μg/mL, and aluminum hydroxide at a final concentration of about 1 mg/mL, wherein the pH of the injectable formulation is about 7.4.

The disclosure also provides and injectable formulation in a multiple unit dose vial containing about 1 mL wherein the injectable formulation comprises an immunogenic fusion protein of SEQ ID NO: 43 at a final concentration of about 120 μg/mL, sodium phosphate buffer at final concentration of about 9 mM, NaCl at a final concentration of about 139 mM, polysorbate 20 at a final concentration of about 275 μg/mL, and aluminum hydroxide at a final concentration of about 1 mg/mL, wherein the pH of the injectable formulation is about 7.4.

The disclosure also provides and injectable formulation in a multiple unit dose vial containing about 1 mL wherein the injectable formulation comprises an immunogenic fusion protein of SEQ ID NO: 43 at a final concentration of about 180 μg/mL, sodium phosphate buffer at final concentration of about 9 mM, NaCl at a final concentration of about 139 mM, polysorbate 20 at a final concentration of about 275 μg/mL, and aluminum hydroxide at a final concentration of about 1 mg/mL, wherein the pH of the injectable formulation is about 7.4.

Optimal amounts of components for a particular immunogenic composition can be ascertained by standard studies involving observation of appropriate immune responses in subjects. For example, in another embodiment, the dosage for human vaccination is determined by extrapolation from animal studies to human data. In another embodiment, the dosage is determined empirically.

In some embodiments, the pharmaceutical composition is delivered in a controlled release system. For example, the agent can be administered using intravenous infusion, a transdermal patch, liposomes, or other modes of administration. In another embodiment, polymeric materials are used; e.g. in microspheres in or an implant.

In certain embodiments, the compositions of the invention are administered to a subject by one or more methods known to a person skilled in the art, such as parenterally, transmucosally, transdermally, intramuscularly, intravenously, intra-dermally, intra-nasally, subcutaneously, intra-peritoneally, and formulated accordingly. In one embodiment, compositions of the present invention are administered via epidermal injection, intramuscular injection, intravenous, intra-arterial, subcutaneous injection, or intra-respiratory mucosal injection of a liquid preparation. Liquid formulations for injection include solutions and the like.

### III. METHODS OF USE

These fusion proteins disclosed herein comprising two or more distinct immunogenic polypeptides represent a novel, cost effective, way to improve vaccine efficacy. The CbpA, cytolysoid fusion proteins provided herein (such as those examples provided in Tables 1 and 2) are immunogenic and depending on the design of the fusion protein and the choice of the polypeptide components, they find use in the treatment and prevention of a variety of bacterial infections.

The compositions provided herein find use in methods for preventing and treating bacterial infections. As used herein, “preventing a bacterial infection” is intended administration of a therapeutically effective amount of an immunogenic fusion protein, immunogenic composition, or vaccine provided herein to an animal in order to protect the animal from the development of a bacterial infection or the symptoms thereof. In some embodiments, a composition presented herein is administered to a subject, such as a human, that is at risk for developing a bacterial infection. By “treating a bacterial infection” is intended administration of a therapeutically effective amount of a fusion protein, immunogenic composition, or vaccine provided herein to an animal that has a bacterial infection or that has been exposed to a bacterium, where the purpose is to cure, heal, alleviate, relieve, alter, remedy, ameliorate, improve, or affect the condition or the symptoms of the bacterial infection.

A “therapeutically effective amount” as used herein refers to that amount which provides a therapeutic effect for a given condition and administration regimen. Thus, the phrase “therapeutically effective amount” is used herein to mean an amount sufficient to cause an improvement in a clinically significant condition in the host. In particular aspects, a “therapeutically effective amount” refers to an amount of an immunogenic fusion protein, immunogenic composition, or vaccine provided herein that when administered to an animal brings about a positive therapeutic response with respect to the prevention or treatment of a subject for a bacterial infection. A positive therapeutic response with respect to preventing a bacterial infection includes, for example, the production of antibodies by the subject in a quantity sufficient to protect against development of the disease. Similarly, a positive therapeutic response in regard to treating a bacterial infection includes curing or ameliorating the symptoms of the disease. In the present context, a deficit in the response of the host can be evidenced by continuing or spreading bacterial infection. An improvement in a clinically significant condition in the host includes a decrease in bacterial load, clearance of bacteria from colonized host cells, reduction in fever or inflammation associated with infection, or a reduction in any symptom associated with the bacterial infection.

In particular aspects, methods for preventing a pneumococcal infection in an animal comprise administering to the animal a therapeutically effective amount of an immunogenic fusion protein disclosed herein, an immunogenic composition comprising an immunogenic fusion protein disclosed herein in combination with a pharmaceutically acceptable carrier, or a vaccine disclosed herein, thereby preventing a pneumococcal infection. When treating or preventing pneumococcal infections, at least one of the various immunogenic fusion proteins comprising at least one polypeptide from pneumococcus will be used (e.g., a CbpA fusion protein, a fusion peptide from any other immunogenic pneumococcal protein or a pneumolysoid fusion protein, as discussed elsewhere herein). In other embodiments, methods for treating a pneumococcal infection in an animal infected with or exposed to a pneumococcal bacterium comprise administering to the animal a therapeutically effective amount of a fusion protein, an immunogenic composition comprising a fusion protein in combination with a pharmaceutically acceptable carrier, or a vaccine disclosed herein, thereby treating the animal. For example, in an individual already infected with a pneumococcal bacterium, an immunogenic fusion protein provided herein could be used as protection against the spread of the infection from the blood to the brain.

A method of inducing an immune response in a subject which has been exposed to or infected with a pneumococcal bacterium is further provided comprising administering to the subject a therapeutically effective amount of an immunogenic fusion protein provided herein (i.e., such as the fusion proteins listed in Tables 1 or 2), or a biologically active variant or fragment thereof, an immunogenic composition, or a vaccine as disclosed herein, thereby inducing an immune response.

Pneumococcal infection involves bacterial colonization of nasopharyngeal epithelial cells and subsequent bacterial entry into the bloodstream, lungs, and, possibly, the brain. While not being bound by any theory, CbpA mediated binding to pIgR and the laminin receptor contribute to nasopharyngeal colonization and invasion into the bloodstream and the brain. The two binding activities have been localized to specific regions of the R2 domain of CbpA. In particular, the R2₁ region is responsible for binding to pIgR and bacterial colonization in the nasopharynx and invasion via transcytosis, whereas the R2₂ region is involved in binding to the laminin receptor and subsequent bacterial invasion of brain and other host tissues. This information can be utilized to develop immunogenic compositions and vaccines that are protective against both steps of pneumococcal infection, namely colonization of the nasopharynx and bacterial entry into the bloodstream.

In some embodiments, a fusion protein comprising, but not limited to, a CbpA polypeptide, or a biologically active variant or fragment thereof, can be employed in various methods to decrease pneumococcal colonization of the nasopharynx (i.e. a fusion protein comprising the R2₁ region of SEQ ID NOS: 1 or 3 or an active variant or fragment thereof, wherein the R2₁ region is in the loop conformation) or to decrease bacterial entry into the bloodstream and brain (i.e. a fusion protein comprising the R2₂ region of SEQ ID NOS: 2 or 4 or an active variant or fragment thereof, wherein said R2₂ region is in the loop conformation), or in other embodiments, can be used to decrease bacterial entry into the lung, into the bloodstream or across the blood brain barrier (i.e. a fusion protein comprising both an R2₁ and R2₂ sequence such as those sequences of (SEQ ID NOS: 1, 2, 3, 4, 41 or 42 or active variants or fragments thereof, wherein the R2₁ and/or the R2₂ are in the loop conformation). As used herein a “decrease” is meant at least a 1%, 5%, 10%, 20%, 30%, 40%, 50%, 60%, 70%, 80%, or 90% decrease relative to an appropriate control, or alternatively, decreased to a sufficient level to produce a desired therapeutic effect in the animal. Various methods to measure bacterial colonization are known in the art. For example, bacteria in the blood can be measured by taking a blood sample and spreading the blood out on an agar plate which contains the appropriate medium for bacterial growth. Bacteria in the nasopharynx can be measured by culturing bacteria from a swab or lavage of the nasopharynx of an animal. Bacteria that have crossed the blood brain barrier can be measured in a sample of cerebrospinal fluid or by detecting the physical attributes of meningitis in an animal, such as spinning.

Embodiments of the invention also include one or more of the immunogenic fusion proteins described herein (i) for use in, (ii) for use as a medicament or composition for, or (iii) for use in the preparation of a medicament for: (a) therapy (e.g., of the human body); (b) medicine; (c) inhibition of infection with Streptococcus pneumoniae, (d) induction of an immune response or a protective immune response against S. pneumoniae, (e) prophylaxis of infection by S. pneumoniae, (1) prevention of recurrence of S. pneumoniae infection; (g) reduction of the progression, onset or severity of pathological symptoms associated with S. pneumoniae infection including the prevention of associated complications such as brain damage, hearing loss, and seizures, (h) reduction of the likelihood of a S. pneumoniae infection or, (i) treatment, prophylaxis of, or delay in the onset, severity, or progression of pneumococcal disease(s), including, but not limited to: pneumococcal pneumonia, pneumococcal bacteremia, pneumococcal meningitis, otits media and sinusitis. In these uses, the immunogenic fusion protein compositions of the invention can optionally be employed in combination with one or more adjuvants, or without an adjuvant.

Accordingly, the invention provides methods for the prophylactic treatment of (i.e. protection against) S. pneumoniae infection or pneumococcal disease comprising administering one or more of the immunogenic fusion protein compositions of the invention to a patient in need of treatment.

The compositions and formulations of the present invention can be used to protect or treat a human susceptible to infection, e.g., a pneumococcal infection, by means of administering such composition or formulation via a systemic or mucosal route.

In one embodiment, the invention provides a method of inducing an immune response to S. pneumoniae, comprising administering to a patient an immunologically effective amount of an immunogenic fusion protein of the invention. In another embodiment, the invention provides a method of vaccinating a human against a pneumococcal infection, comprising the step of administering to the human an immunologically effective amount of an immunogenic fusion protein composition of the invention.

Thus, in one aspect, the invention provides a method for (1) inducing an immune response in a human patient, (2) inducing a protective immune response in a human patient, (3) vaccinating a human patient against an infection with S. pneumoniae, or (4) reducing the likelihood of a S. pneumoniae infection in a human patient and the method comprising administering a immunogenic fusion protein composition of the invention to the patient.

In one embodiment, the invention provides a method for the prevention of pneumococcal pneumonia and/or invasive pneumococcal disease in an infant (less than 1 year of age), toddler (approximately 12 to 24 months), or young child (approximately 2 to 5 years). In another embodiment, the invention provides a method for the prevention of pneumococcal pneumonia and/or invasive pneumococcal disease in a 6 month through 17 year old patient. In another embodiment, the invention provides a method for the prevention of pneumococcal pneumonia and/or invasive pneumococcal disease in adults 18 years of age and older. In another embodiment, the invention provides a method for the prevention of pneumococcal pneumonia and/or invasive pneumococcal disease in adults 50 years of age and older. In another embodiment, the invention provides a method for the prevention of pneumococcal pneumonia and/or invasive pneumococcal disease in adults 65 years of age and older.

The invention provides a method of inducing an immune response, vaccinating, or inducing a protective immune response against S. pneumoniae in a patient, comprising administering an immunogenic fusion protein composition to the patient, wherein the patient had previously been vaccinated against S. pneumoniae. In embodiments of this aspect of the invention, the immunogenic composition can be any immunogenic fusion protein composition described herein.

In additional embodiments of the method above, the patient was previously treated with PREVNAR® 13 (Pneumococcal 13-valent Conjugate Vaccine [Diphtheria CRM197 Protein], Pfizer, Inc., Philadelphia, PA, USA). In further embodiments of the method above, the patient was previously treated with PNEUMOVAX® 23 (Pneumococcal Vaccine Polyvalent, Merck & Co., Inc., Kenilworth, NJ, USA), SYNFLORIX™ (Pneumococcal polysaccharide conjugate vaccine (adsorbed), GlaxoSmithKline Biologicals s.a., Rixensart, Belgium), PREVNAR 20™ (20 valent conjugate vaccine; Pfizer), VAXNEUVANCE™ (valent; Merck), or any combination thereof.

In yet another embodiment, an immunogenic fusion protein provided herein can be employed in various methods to treat and prevent Neisseria meningitidis infection. Neisseria meningitidis is another bacterium that crosses the blood brain barrier and causes meningitis. As disclosed in U.S. Patent Publication No. 2010-0143394-A1, herein incorporated by reference, Neisseria meningitidis binds to the laminin receptor to cross the blood brain barrier. This is the same mechanism used by Streptococcus pneumoniae. Disclosed herein are fusion proteins comprising, but not limited to, the R2₁ or R2₂ regions, R2₁ or R2₂ regions having loop conformations or active variants or fragments thereof, of CbpA can cross-protect against Neisseria meningitidis. Therefore, the fusion proteins provided herein have use as a vaccine for the treatment and prevention of infections of other bacteria that utilize similar infectious mechanisms.

A fusion protein comprising a cytolysoid can be employed in various methods to treat and prevent bacterial infections. As discussed above, the cytolysoid polypeptides (or active variant or fragment thereof) can be modified from any bacterial cytolysin and be employed to create a fusion protein with one or more immunogenic polypeptides from the same bacterial source or a different bacterial source as the cytolysoid. In this way, methods to treat and prevent various bacterial infections are encompassed herein. Some examples of bacteria that may cause bacterial infections are disclosed elsewhere herein.

The immunogenic fusion proteins provided herein could also be used in various methods to treat or prevent multiple bacterial infections in an animal. The immunogenic fusion proteins could comprise a combination of immunogenic polypeptides from two or more bacteria. In a particular aspect, the immunogenic polypeptides of the fusion protein would originate from bacterial sources that are frequently found simultaneously in a given animal. For example, infections caused by Streptococcus pneumoniae and Haemophilus influenzae, which can simultaneously infect the nasopharynx, could be treated or prevented by a fusion protein comprising immunogenic polypeptides from both bacteria.

### IV. METHODS OF ADMINISTRATION

The immunogenic fusion proteins (e.g. MTRV001), vaccines, compositions and formulations provided herein can be administered via any parenteral route, which include but not limited to intravenous, intramuscular, subcutaneous, intraperitoneal, intradermal, oral (e.g., inhalation), transdermal (i.e., topical), transmucosal, and rectal administration. In some embodiments, the immunogenic fusion protein is administered intramuscularly. Preferably, since the desired result of the administration is to elucidate an immune response to the antigen, and thereby to the pathogenic organism, administration directly, or by targeting or choice of a viral vector, indirectly, to lymphoid tissues, e.g., lymph nodes or spleen, is desirable. Since immune cells are continually replicating, they are ideal targets for retroviral vector-based nucleic acid vaccines, since retroviruses require replicating cells.

It will be appreciated that administration of therapeutic entities in accordance with the invention will be administered with suitable carriers, excipients, and other agents that are incorporated into formulations to provide improved transfer, delivery, tolerance, and the like as described above. A multitude of appropriate formulations can be found in the formulary known to all pharmaceutical chemists: Remington's Pharmaceutical Sciences (15th ed., Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

### A. Dosages Regimens

A subject in whom administration of an active component as set forth above is an effective therapeutic regimen for a bacterial infection is preferably a human, but can be any animal. Thus, as can be readily appreciated by one of ordinary skill in the art, the methods and pharmaceutical compositions provided herein are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., i.e., for veterinary medical use.

In the therapeutic methods and compositions provided herein, a therapeutically effective dosage of the active component is provided. A therapeutically effective dosage can be determined by the ordinary skilled medical worker based on patient characteristics (age, weight, sex, condition, complications, other diseases, etc.), as is well known in the art. Furthermore, as further routine studies are conducted, more specific information will emerge regarding appropriate dosage levels for treatment of various conditions in various patients, and the ordinary skilled worker, considering the therapeutic context, age and general health of the recipient, is able to ascertain proper dosing. Generally, for intravenous injection or infusion, dosage may be lower than for intraperitoneal, intramuscular, or other route of administration. The dosing schedule may vary, depending on the circulation half-life, and the formulation used. The compositions are administered in a manner compatible with the dosage formulation in the therapeutically effective amount. Precise amounts of active ingredient required to be administered depend on the judgment of the practitioner and are peculiar to each individual. However, suitable dosages may range from about 0.1 mg/kg to 20 mg/kg, preferably about 0.5 mg/kg to about 10 mg/kg, and more preferably one to several, milligrams of active ingredient per kilogram body weight of individual per day and depend on the route of administration. Common ranges for therapeutically effective dosing of the immunogenic fusion protein of the invention may be, by way of nonlimiting example, from about 0.1 mg/kg body weight to about 50 mg/kg body weight. Preferred doses may include 1, 3, 6, 10 mg/kg body weight. Common dosing frequencies may range, for example, from once monthly. Treatment may last 2, 3, 4, 5, 6, 7, 8, 9, 10, 11, 12 or more months. Suitable regimens for initial administration and booster shots are also variable, but are typified by an initial administration followed by repeated doses at one or more hour intervals by a subsequent injection or other administration. Alternatively, continuous injections (e.g., subcutaneous or intramuscular) sufficient to maintain concentrations of ten nanomolar to ten micromolar in the blood are contemplated.

The disclosure provides a method of treating, prophylactically preventing, or reducing the occurrence of a condition, disease, or infection caused by Streptococcus pneumoniae, in a subject in need thereof comprising administering to the subject at least one dose of a composition comprising an immunogenic fusion protein (e.g. MTRV001). In some embodiments, a dose of the immunogenic fusion protein comprises about 5 μg to about 150 μg. In some embodiments, a dose of the immunogenic fusion protein comprises about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 105 μg, about 110 μg, about 115 μg, about 120 μg, about 125 μg, about 130 μg, about 140 μg, about 145 μg or about 150 μg. In some embodiments, the dose of the immunogenic fusion protein is about 10 μg. In some embodiments, the dose of the immunogenic fusion protein is about 30 μg. In some embodiments, the dose of the immunogenic fusion protein is about 60 μg. In some embodiments, the dose of the immunogenic fusion protein is about 90 μg.

In some embodiments, the composition comprising a dose of the immunogenic fusion protein is administered in at least one dose. In some embodiments, the composition comprising a dose of the immunogenic fusion protein is administered in no more than two doses, in no more than three doses, in no more than four doses or no more than five doses. In some embodiments, the composition comprising a dose of the immunogenic fusion protein is administered in no more than two doses. In some embodiments, the composition comprising a dose of the immunogenic fusion protein is administered in two doses.

In some embodiments, the dose is administered in at least a first dose and a second dose. In some embodiments the first dose is higher than the second dose. In some embodiments, the second dose is higher than the first dose. In some embodiments, the first dose and the second dose are equal.

In some embodiments, the amount of time between each dose is from about 4 weeks to about one year. In some embodiments, the amount of time between each dose is one week, two weeks, three weeks, four weeks, five weeks, six weeks, seven weeks or eight weeks. In some embodiments, the amount of time between each dose is 2 months, 3 months, 4 months, 5 months, 6 months, 7 months, 8 months, 9 months, 10 months, 11 months, 12 months, 13 months, 14 months or 15 months. In some embodiments, the second dose is administered 14, 15, 16, 17, 18, 19, 20, 21, 22, 23, 24, 25, 26, 27, 28, 29, 30, 31, 32, 33, 34 or 35 days after the first dose. In some embodiments, the second dose is administered 28 days after the first dose.

B. Administration with other compounds. For treatment of a bacterial infection, one may administer the present active component in conjunction with one or more pharmaceutical compositions used for treating bacterial infection, including but not limited to (1) antibiotics; (2) soluble carbohydrate inhibitors of bacterial adhesin; (3) other small molecule inhibitors of bacterial adhesin; (4) inhibitors of bacterial metabolism, transport, or transformation; (5) stimulators of bacterial lysis, or (6) anti-bacterial antibodies or vaccines directed at other bacterial antigens. Other potential active components include anti-inflammatory agents, such as steroids and non-steroidal anti-inflammatory drugs. Administration may be simultaneous (for example, administration of a mixture of the present active component and an antibiotic) or may be in seriatim.

### V. METHODS OF MANUFACTURE

For recombinant production of an immunogenic fusion protein of the invention, the nucleic acid encoding it is isolated and inserted into a replicable vector for further cloning (amplification of the DNA) or for expression. DNA encoding the immunogenic fusion protein is readily isolated and sequenced using conventional procedures (e.g., by using oligonucleotide probes that are capable of binding specifically to genes encoding the immunogenic fusion protein). Many vectors are available. The choice of vector depends in part on the host cell to be used. Generally, preferred host cells are of either prokaryotic or eukaryotic (generally mammalian, but also including fungi (e.g., yeast), insect, plant, and nucleated cells from other multicellular organisms) origin.

This method provides a method of purifying an immunogenic fusion protein (e.g. MTRV001) comprising a) providing a vector comprising an nucleic acid encoding the polypeptide; b) introducing the vector into a population of host cells; c) culturing the population of host cells under conditions that allow for expression of the polypeptide; d) disrupting the cell membranes of the host cells; and e) recovering the polypeptide. The method may further comprise at least one purification step comprising contacting the polypeptide with a separation means, eluting the polypeptide from the separation means under conditions that allow for preferential detachment of the polypeptide. The method may further comprise a filtration step comprising contacting the eluted polypeptide with a filter.

### A. Generating Immunogenic Fusion Proteins Using Prokaryotic Host Cells

Polynucleotide sequences encoding polypeptide components of the immunogenic fusion protein of the invention can be obtained using standard recombinant techniques. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular immunogenic fusion proteins are described in detail in Carter et al., U.S. Pat. No. 5,648,237.

The expression vector of the invention may comprise two or more promoter-cistron pairs, encoding each of the polypeptide components. A promoter is an untranslated regulatory sequence located upstream (5′) to a cistron that modulates its expression.

Prokaryotic promoters typically fall into two classes, inducible and constitutive. An inducible promoter is a promoter that initiates increased levels of transcription of the cistron under its control in response to changes in the culture condition, e.g., the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the T7 promoter, PhoA promoter, the β-galactosidase and lactose promoter systems, a tryptophan (tip) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker to ligate them to cistrons encoding the target light and heavy chains (Siebenlist et al., (1980) Cell 20:269) using linkers or adaptors to supply any required restriction sites.

Prokaryotic host cells suitable for expressing immunogenic fusion proteins of the invention include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In one embodiment, Gram-negative cells are used. In one embodiment, E. coli cells are used as hosts for the invention. Examples of E. coli strains include strain HMS174 (DE3), strain W3110 (Bachmann, Cellular and Molecular Biology, vol. 2 (Washington, D.C.: American Society for Microbiology, 1987), pp. 1 190-1219; ATCC Deposit No. 27,325) and derivatives thereof, including strain 33D3 having genotype W31 10 AfhuA (AtonA) ptr3 lac Iq lacL8 AompTA(nmpc-fepE) degP41 kanR (U.S. Pat. No. 5,639,635). Other strains and derivatives thereof, such as E. coli 294 (ATCC 31,446), E. coli B, E. coli λ 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins 8:309-314 (1990). It is generally necessary to select the appropriate bacteria taking into consideration replicability of the replicon in the cells of a bacterium. For example, E. coli, Serratia, or Salmonella species can be suitably used as the host when well-known plasmids such as pBR322, pBR325, pACYC 177, or pKN410 are used to supply the replicon. Typically the host cell should secrete minimal amounts of proteolytic enzymes or other contaminants, and additional protease inhibitors may desirably be incorporated in the cell culture.

In some embodiments, the immunogenic fusion protein of the invention is cloned into an E. coli expression vector. In some embodiments, the E. coli expression vector comprises a T7 promoter. In some embodiments, the E. coli expression vector is a pET24a+.

### B. Immunogenic Fusion Protein Production

Host cells are transformed with the above-described expression vectors and cultured in conventional nutrient media modified as appropriate for inducing promoters, selecting transformants, or amplifying the genes encoding the desired sequences.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the invention are grown in media known in the art and suitable for culture of the selected host cells. Examples of suitable media include Luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol, and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the invention, protein expression is induced under conditions suitable for the activation of the promoter. In one aspect of the invention, IPTG is used for controlling expression of the polypeptides. A variety of other inducers may be used, according to the vector construct employed, as is known in the art.

In one embodiment, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by a separation means. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In some embodiments, the separation means is a resin, a membrane, a magnetic bead or a particle.

In some embodiments, the separation means is affinity chromatography. Exemplary affinity chromatography methods include but are not limited to hydrophobic interaction chromatography, anion exchange chromatography, cation exchange chromatography, hydroxyapatite (mixed-mode) chromatography, gel filtration chromatography, size exclusion chromatography, hydrophilic interaction chromatography and/or a combination thereof.

In some embodiments, the separation means is a hydrophobic interaction chromatography resin. In some embodiments, the hydrophobic interaction chromatography resin is a Phenyl Sepharose™ FF resin.

In some embodiments, the separation means is an anion exchange chromatography resin. In some embodiments, the hydrophobic interaction chromatography resin is a Q Sepharose™ HP resin.

In some embodiments, the separation means is a combination of at least two separation means. In some embodiments, the separation means is a combination of at least two affinity chromatography resins. In some embodiments, the separation means is a combination of at least three separation means. In some embodiments, the separation means is a combination of at least three affinity chromatography resins. In some embodiments, the separation means is a combination of more than two affinity chromatography resins, e.g., three or more, four or more, and/or five or more affinity chromatography resins.

In some embodiments, the separation means includes the use of an anion exchange chromatography resin followed by the use of a hydrophobic interaction chromatography resin. In some embodiments, the separation means includes the use of Q Sepharose™ HP resin followed by the use of a Phenyl Sepharose™ FF resin.

In some embodiments, the separation means includes the use of a hydrophobic interaction chromatography resin followed by the use of an anion exchange chromatography resin. In some embodiments, the separation mean includes the use of a Phenyl Sepharose™ FF resin followed by the use of a Q Sepharose™ HP resin.

In some embodiments, the separation means includes the use of a hydrophobic interaction chromatography resin, followed by a flow through anion exchange resin, followed by a multi-modal (hydroxyapatite) chromatography resin.

In some embodiments, the binding and/or elution conditions include a step variation in the pH level and/or a step variation in conductivity corresponding to salt concentration variation. In some embodiments, the binding and/or elution conditions include a step variation in the inorganic salt concentration such as sodium chloride (NaCl) concentration or the concentration of other inorganic salts such as by way of non-limiting and non-exhaustive example, inorganic salt combinations from the Hofmeister series of ions, for example, a sulfate. In some embodiments, the methods include the step of varying the concentration of ammonium sulfate for binding and/or elution. In some embodiments, the methods do not include the step of varying the concentration of ammonium sulfate for binding and/or elution.

The present disclosure provides a method of producing an immunogenic fusion protein, comprising the steps of: a) culturing a population of the host cells expressing an immunogenic fusion protein comprising the amino acid sequence of SEQ ID NO: 43 in a condition suitable for the population of host cells to produce the immunogenic fusion protein; b) disrupting the cell membranes of the host cells; c) recovering a sample comprising the immunogenic fusion protein and one or more impurities; d) contacting the sample comprising the immunogenic fusion protein with a hydrophobic interaction chromatography resin and eluting the immunogenic fusion protein from the hydrophobic interaction chromatography resin under conditions that allow for preferential detachment of the immunogenic fusion protein, thereby obtaining an eluate comprising the immunogenic fusion protein; e) subjecting the eluate comprising the immunogenic fusion protein of step d) to a flow through anion exchange resin, thereby obtaining an eluate comprising the immunogenic fusion protein; and f) contacting the eluate comprising the immunogenic fusion protein of step e) with a multi-modal chromatography resin and eluting the immunogenic fusion protein from the multi-modal chromatography resin under conditions that allow for preferential detachment of the immunogenic fusion protein, thereby obtaining an eluate comprising the immunogenic fusion protein.

Generally, the samples contain various impurities in addition to the target molecule (e.g., immunogenic fusion protein). Such impurities include media components, cells, cell debris, nucleic acids, host cell proteins (HCP), viruses, endotoxins, etc. Other impurities include non-monomeric forms of the target molecule (e.g., immunogenic fusion protein) or non-full length forms of the target molecule (e.g., N-terminal truncations of the immunogenic fusion protein or C-terminal truncations of the immunogenic fusion protein). All such target molecule related impurities may decrease the immunogenicity and impact the quality of an immune response in a therapeutic application. The methods herein provide a specific order of purification steps to produce compositions with a high purity of immunogenic fusion proteins (e.g. full length, monomeric forms) and a low level of impurifies (e.g. HCP, endotoxins, DNA), suitable for therapeutic applications, which was not previously achievable through means known in the art.

In preferred embodiments, flow-through purification further includes one or more additional flow-through steps, e.g., for aggregate removal and virus filtration. In some embodiments, the sample is passed through an adsorptive depth filter, or a charged or modified microporous layer or layers in a normal flow filtration mode of operation, for aggregate removal. Examples of flow-through steps which may be used for aggregate removal can be found in, e.g., U.S. Pat. Nos. 7,118,675 and 7,465,397, incorporated by reference herein. Accordingly, in some embodiments, a two-step filtration process for removing protein aggregates and viral particles may be used, wherein a sample is first filtered through one or more layers of adsorptive depth filters, charged or surface modified porous membranes, or a small bed of chromatography media to produce a protein aggregate-free sample. This may be followed by the use of an ultrafiltration membrane for virus filtration, as described in more detail below. Ultrafiltration membranes used for virus filtration are typically referred to as nanofiltration membranes

In some embodiments, the methods include a further step of determining the purity of the immunogenic fusion protein in the eluted fraction. This step can be accomplished using any of a variety of art-recognized techniques, such as by way of non-limiting and non-exhaustive example, hydrophobic interaction-high performance liquid chromatography (HIC-HPLC), ion exchange-high performance liquid chromatography (IEX-HPLC), cation exchange-high performance liquid chromatography (CEX-HPLC) or reverse phase-high performance liquid chromatography (RP-HPLC).

In some embodiments, the method further comprises the step of: g) contacting the eluate comprising the immunogenic fusion protein of step f) with a flow through anion exchange membrane; thereby obtaining an eluate comprising the immunogenic fusion protein. In some embodiments, the method further comprises the steps of: h) contacting the eluate comprising the immunogenic fusion protein of step g) with an ultrafiltration/diafiltration membrane; and i) washing the immunogenic fusion protein from the ultrafiltration/diafiltration membrane under conditions that allow for preferential detachment of the immunogenic fusion protein, thereby obtaining an eluate comprising the immunogenic fusion protein. In some embodiments, the method further comprises the step of: j) contacting the eluate comprising the immunogenic fusion protein of step i) with a 0.2 μm filter.

The resulting compositions comprises lower levels of impurities, such as media components, cells, cell debris, nucleic acids, host cell proteins (HCP), viruses, endotoxins, etc. Other impurities include non-monomeric forms of the target molecule (e.g., immunogenic fusion protein) or non-full-length forms of the target molecule (e.g. N-terminal truncations of the immunogenic fusion protein).

In some embodiments, the composition comprising the immunogenic fusion protein (e.g. SEQ ID NO: 43) comprises at least about 90%, at least about 91%, at least about 92%, at least about 93%, at least about 94%, at least about 95%, at least about 96%, at least about 97%, at least about 98%, at least about 99%, or 100% or any percentage in between, of the monomeric form of the immunogenic fusion protein. In some embodiments, the composition comprising the immunogenic fusion protein comprises about 98% of the monomeric form of the immunogenic fusion protein. In some embodiments, the composition comprising the immunogenic fusion protein comprises about 99% of the monomeric form of the immunogenic fusion protein. In some embodiments, the composition comprising the immunogenic fusion protein comprises about 100% of the monomeric form of the immunogenic fusion protein.

In some embodiments, the composition comprising the immunogenic fusion protein (e.g. SEQ ID NO: 43) comprises less than 50 EU/mg, less than 45 EU/mg, less than 30 EU/mg, less than 25 EU/mg, less than 20 EU/mg, less than 10 EU/mg, less than 5 EU/mg, less than 4 EU/mg, less than 3 EU/mg, less than 2 EU/mg, less than 1 EU/mg or less than 0.1 EU/mg of endotoxin per mg of immunogenic fusion protein. In some embodiments, the composition comprises less than 2 EU/mg, less than 1.9 EU/mg, less than 1.8 EU/mg, less than 1.7 EU/mg, less than 1.6 EU/mg, less than 1.5 EU/mg, less than 1.4 EU/mg, less than 1.3 EU/mg, less than 1.2 EU/mg, less than 1.1 EU/mg, less than 1.0 EU/mg, less than 0.9 EU/mg, less than 0.8 EU/mg, less than 0.7 EU/mg, less than 0.6 EU/mg, less than 0.5 EU/mg, less than 0.4 EU/mg, less than 0.3 EU/mg, less than 0.2 EU/mg or less than 0.1 EU/mg of endotoxin per mg of immunogenic fusion protein. In some embodiments, the composition comprises about 17 EU/mg of endotoxin per mg of immunogenic fusion protein. In some embodiments, the composition comprises less than 10 EU/mg of endotoxin per mg of immunogenic fusion protein. In some embodiments, the composition comprises about 1.9 EU/mg of endotoxin per mg of immunogenic fusion protein. In some embodiments, the composition comprises less than 0.1 EU/mg of endotoxin per mg of immunogenic fusion protein.

In some embodiments, the composition comprising the immunogenic fusion protein comprises less than 80000 ng/mg, less than 75000 mg/mg, less than 70000 ng/mg, less than 65000 ng/mg, 60000 ng/mg, less than 55000 mg/mg, less than 50000 ng/mg, less than 45000 ng/mg, 40000 ng/mg, less than 35000 mg/mg, less than 30000 ng/mg, less than 25000 ng/mg, 20000 ng/mg, less than 15000 mg/mg, less than 10000 ng/mg, less than 5000 ng/mg, less than 4000 ng/mg, less than 3000 mg/mg, less than 2000 ng/mg, less than 1000 ng/mg, less than 900 ng/mg, less than 800 ng/mg, less than 700 ng/mg, less than 600 ng/mg, less than 500 ng/mg less than 400 ng/mg, less than 300 ng/mg, less than 200 ng/mg or less than 100 ng/mg of host cell protein (HCP) per mg of immunogenic fusion protein. In some embodiments, the composition comprising the immunogenic fusion protein comprises less than 90 ng/mg, less than 80 ng/mg, less than 70 ng/mg, less than 60 ng/mg, less than 50 ng/mg less than 40 ng/mg, less than 30 ng/mg, less than 20 ng/mg or less than 10 ng/mg, or any value in between, of host cell protein (HCP) per mg of immunogenic fusion protein. In some embodiments, the composition comprising the immunogenic fusion protein comprises about less than 200 ng/mg of host cell protein (HCP) per mg of immunogenic fusion protein. In some embodiments, the composition comprising the immunogenic fusion protein comprises about 76,600 ng/mg of host cell protein (HCP) per mg of immunogenic fusion protein. In some embodiments, the composition comprising the immunogenic fusion protein comprises about 30 ng/mg of HCP per mg of immunogenic fusion protein.

In one aspect of the invention, immunogenic fusion protein production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small-scale fermentation refers generally to fermentation in a fermentor that is no more than approximately 100 liters in volumetric capacity and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease HI, OmpT, DegP, Tsp, Protease I, Protease Mi, Protease V, Protease VI, and combinations thereof. Some E. coli protease-deficient strains are available and described in, for example, Joly et al., (1998), Proc. Natl. Acad. Sci. USA 95:2773-2777; Georgiou et al., U.S. Pat. No. 5,264,365; Georgiou et al., U.S. Pat. No. 5,508,192; Hara et al., Microbial Drug Resistance, 2:63-72 (1996). In one embodiment, E. coli strains deficient for proteolytic enzymes and transformed with plasmids overexpressing one or more chaperone proteins are used as host cells in the expression system of the invention.

### VI. EXAMPLES

The following examples are provided by way of illustration, not by way of limitation. All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

### Example 1: Construction and Pharmacodynamic Study of MTRV001

### Overview

Disclosed herein is a pneumococcal vaccine candidate for the active immunization for prevention of pneumonia and invasive disease caused by Streptococcus pneumoniae. The detailed in the pharmacodynamic and toxicology summaries, supports the evaluation of MTRV001 in the proposed Phase 1 clinical study.

### Construction of MTRV001

As depicted by the schematic in FIGS. 1A-1B, MTRV001 is a 520 amino acid fusion protein consisting of a genetically detoxified pneumolysin (PLY) component with conserved peptide sequences derived from choline binding protein A (CbpA) fused to the amino- and carboxy-termini of the PLY protein.

The PLY component (470 amino acids from serotype 4 S. pneumoniae strain TIGR4) of MTRV001 includes two highly attenuating amino acid substitutions (G293S and L460D) that abrogate the cytolytic activity of native PLY, as depicted in FIG. 1A. The G293S mutation locks the protein in a pre-pore confirmation which inhibits oligomerization of the PLY molecules and results in a soluble, monomeric protein (Oloo, 2011). The L460D mutation is intended to prevent cholesterol binding (Farrand, 2010), a critical aspect of PLY pore forming activity.

The CbpA components of MTRV001 are derived from the R2 domain (2nd repeat domain) of the S. pneumoniae strain TIGR4 native protein. A schematic representation of the R2 domain of CbpA is depicted in FIG. 2A. The R2 domain is comprised of 12 imperfect copies of the leucine zipper motif (Luo, 2005). The highly conserved loops between antiparallel helices 1 and 2, and 2 and 3, are important for binding to epithelial polymeric immunoglobulin receptor and binding to the laminin-specific integrin receptor, respectively (Mann, 2014). As shown in FIG. 2A, the CbpA-Y sequence (31 amino acids) contains the highly conserved RRNYPT from the Helix 1-Helix 2 loop. The CbpA-N sequence (17 amino acids) contains the highly conserved sequence EPRNEEK from the Helix 2-Helix 3 loop. Two nonhelical loop regions (FIG. 2A; boxes) link the 3 antiparallel α-helices. The RRNYPT motif binds to the pIgR receptor on epithelial cells, and the EPRNEEK motif binds to laminin receptor of endothelial cells. Amino acid numbers of the R2 domain are indicated in FIG. 2A. The percentage conservation of sequence from 30 clinical isolates is listed. As depicted in FIG. 2B, regions of R2 were expressed as wildtype (referred to as linear: L-YPT-long, L-NEEK-long: 62 and 82 amino acids, respectively) or dual Cys-containing (YPT-long or NEEK-long) polypeptides made by substituting cysteine residues as indicated (referred to as “looped”). Cysteine residues have been engineered into the CbpA sequences to promote disulfide bridge formation and mimic the native structural confirmation of the CbpA loops (Mann, 2014).

### General Properties of MTRV001

MTRV001 is a fusion protein consisting of a detoxified pneumolysin (PLY) with conserved peptide sequences of the choline binding protein A (CbpA) at the amino- and carboxy-termini. Two attenuating mutations in the PLY sequence, G293S and L460D, are intended to abrogate cytolytic activity by locking PLY in a monomeric, pre-pore confirmation, and prevent cholesterol binding, respectively. It has been shown that the pre pore conformation enables functional antibodies that neutralize PLY toxin cytolytic activity but lack hemolytic activity (Oloo, 2011). The N-terminal CbpA moiety (CbpA-Y) is responsible for CbpA mediated binding to the human epithelial polymeric immunoglobulin receptor and the C terminal CbpA moiety (CbpA-N) binds to the laminin-specific integrin receptor.

TABLE 5
General Properties of MTRV001 Drug Substance

| Physical/Chemical Properties | Description |
|---|---|
| Physical description | Clear, colorless liquid |
| Theoretical extinction coefficient | 1.29 (mg/mL)⁻¹ cm⁻¹ |
| Theoretical pI | 5.2 |
| Target concentration | 1.0 ± 0.2 mg/mL |
| Formulation buffer | 10 mM sodium phosphate, 154 mM sodium chloride, |
| | 275 μg/mL polysorbate 20, pH 7.4 |

During the initial development of MTRV001, a PLY genetic toxoid containing a unique single amino acid substitution, L460D (“PLY-SM”), that disrupted the ability of PLY to effectively bind cholesterol in membranes and lyse cells (Farrand, 2010), was evaluated in development studies. PLY-SM, however, retained residual levels of cytolytic activity and therefore, additional attenuating constructs were developed. As depicted in FIG. 1B, a second PLY genetic toxoid (“PLY-DM”) was evaluated which harbors two amino acid substitutions, G293S and L460D, which lock the protein in a pre pore conformation and prevent cholesterol binding, respectively, and together reduce cytolytic activity to undetectable levels (Farrand, 2010; Oloo, 2011; Thanawastien, 2021). In murine immunogenicity studies, PLY-DM elicited a comparable immune response to that of PLY-SM with respect to anti-PLY IgG titers, functional antibody levels as measured in an in vitro hemolysis neutralization assay, and in conferring protection to mice in an intranasal (IN) S. pneumoniae challenge sepsis model. Subsequently, it demonstrated that immunization of mice with PLY-DM conferred broad and significant protection against lethal IN challenges with 17 of 20 S. pneumoniae serotypes, that included both Prevnar 13@serotypes as well as emerging serotypes (Thanawastien, 2021). Since PLY-DM contained the additional G293 S mutation that further attenuated PLY cytolytic activity while still eliciting high-titer functional antibodies, it was selected as the PLY toxoid component that comprises the final MTRV001 construct.

PLY-SM is a PLY genetic toxoid harboring a single amino acid (aa) attenuating mutation (L460D) (FIG. 1B). PLY-SM was exploited as a template to generate both i) YLN, a recombinant fusion antigen with CbpA peptides flanking the N- and C-termini and ii) PLY-DM, a highly attenuated PLY toxoid harboring two (2) aa substitutions (G293S and L460D). PLY-DM was subsequently used to generate MTRV001, a fusion construct of PLY-DM with flanking N- and C-terminal CbpA peptides, as depicted in FIG. 1B.

“YLN”, a protein that varies by one amino acid from MTRV001, consists of “PLY-SM” as the core antigen and two conserved peptides from the S. pneumoniae cell surface adhesin CbpA fused to its N- and C-termini (Mann, 2014) (FIG. 1B). The CbpA-Y and CbpA-N peptide sequences of YLN originate from the R2 domain (2nd repeat domain) of the native CbpA protein. The CbpA-Y sequence (31 amino acids) contains the highly conserved RRNYPT from the Helix 1-Helix 2 loop and the CbpA-N sequence (17 amino acids) contains the highly conserved sequence EPRNEEK from the Helix 2-Helix 3 loop. In the native protein, the CbpA-Y sequence is responsible for CbpA-mediated binding to the human epithelial polymeric immunoglobulin receptor, whereas the CbpA-N sequence binds to the laminin-specific integrin receptor. In murine studies, YLN was highly immunogenic and conferred superior protection compared to PLY-SM in pneumococcal models of infection, including otitis media and meningitis. Furthermore, YLN immunized mice exhibited significantly less lung pathology following pneumococcal challenge infection than PLY-SM immunized and unimmunized animals.

Based on the published YLN preclinical immunogenicity and efficacy results, MTRV001 was constructed using PLY-DM as the core toxoid component fused to the identical CbpA peptides found in YLN at the N- and C-termini of the toxoid. Preclinical murine immunogenicity and challenge studies demonstrated that MTRV001 elicited comparable anti-PLY immunoglobulin G (IgG) antibody titers and protective efficacy as PLY-DM. Similar to YLN, MTRV001 immunized mice exhibited no observable lung pathology following virulent pneumococcal challenge whereas the lungs of mice immunized with PLY-DM or administered saline exhibited active signs of inflammation and pathology. These data clearly demonstrate the additional protection conferred by the CbpA epitope(s).

MTRV001 was subsequently evaluated for safety, tolerability, and immunogenicity in a GLP repeat-dose toxicity study in rabbits. In the toxicology study, rabbits were administered three injections of 10, 30, or 90 μg of MTRV001 intramuscularly (IM) every two weeks. MTRV001 was well tolerated and demonstrated no evidence of toxicology at any dose level evaluated. Additionally, MTRV001 was immunogenic in a dose-dependent fashion.

### Pharmacology Studies

Primary pharmacology studies were conducted to evaluate the immunogenicity and efficacy of MTRV001 as well as the development construct, PLY-DM. In accordance with World Health Organization (WHO) guidelines, local tolerance was evaluated in the repeat-dose toxicity study. Nonclinical immunogenicity studies of MTRV001 were conducted in both mice and rabbits whereas efficacy studies were performed in murine models of pneumococcal disease. Collectively, these nonclinical studies demonstrate the safety, tolerability, immunogenicity, and protective capacity of MTRV001 for the prevention of pneumonia and invasive disease caused by S. pneumoniae.

MTRV001 efficacy against virulent pneumococcal bacterial challenge was evaluated in a series of murine studies. These studies not only demonstrated that mice immunized with MTRV001 were protected from challenge with three S. pneumoniae serotypes, but also served to bridge the MTRV001 immunogenicity and efficacy data to the precursor antigen PLY-DM. MTRV001 and PLY-DM elicited highly comparable levels of anti-PLY IgG antibodies and conferred comparable levels of protection against both WT PLY toxin challenge and lethal IN S. pneumoniae challenge. The presence of CbpA epitopes in MTRV001 did not improve protection in this bacterial challenge model despite eliciting antibodies to CbpA. This apparent lack of added protection from the CbpA peptides of MTRV001, however, is likely due to the reduced role CbpA plays in this lethal murine sepsis model.

To demonstrate the contribution of the MTRV001 CbpA epitope(s) in mitigating S. pneumoniae pathology, murine studies were conducted comparable in design to those performed with YLN (Mann, 2014). Following immunization of groups of mice with either MTRV001, PLY-DM, or sterile saline, animals were challenged intratracheally (IT) with a pneumococcal serotype 4 strain to ensure delivery of the bacteria into the lungs. As expected, following immunization, MTRV001 and PLY-DM elicited similar levels of anti-PLY IgG antibodies whereas only MTRV001 elicited anti-CbpA IgG antibodies. While mice immunized with MTRV001 and PLY-DM showed similar survival rates following infection, the MTRV001 immunized mice had vastly improved lung pathology compared to PLY-DM and sterile saline immunized mice, as shown in Table 6. These data demonstrate an important role for the CbpA epitope(s) in prevention of pneumococcal-induced lung pathology. While the reduced lung pathology in MTRV001 immunized mice did not translate into improved survival, this is likely due to the mechanism of death, which occurs primarily via bacteremia and sepsis rather than impairment of lung function. Since CbpA functions as an adhesin, it is anticipated to play a more important role in pneumococcal colonization of the upper airways and invasion of tissues. Additionally, the role of CbpA in murine models of S. pneumoniae disease may be reduced given that CbpA-mediated invasion via the polymeric immunoglobulin receptor is specific for the human receptor. Thus, the anticipated contribution of the MTRV001 CbpA epitopes in the prevention of pneumococcal disease will not be realized until human clinical studies (Mann, 2014).

TABLE 6
Lung Pathology Results following Intratracheal Infection of Mice Immunized with
MTRV001, PLY-DM, or Sterile Saline with a Serotype 4 S. pneumoniae Strain

| | | MTRV001 | | |
|---|---|---|---|---|
| Treatment | Severity | (N = 5) | PLY-DM (N = 5) | Saline (N = 5) |
| Lung inflammation, mixed | Minimal | 0 | 3 | 3 |
| cell, alveolus, interstitium | Mild | 0 | 1 | 1 |

The impact of Alhydrogel® as an MTRV001 adjuvant was evaluated in a nonclinical study comparing no adjuvant and Alhydrogel®. BALB/c mice were immunized three times every two weeks with 2 μg and 0.2 μg MTRV001 dose levels. The preclinical immunogenicity ELISA data from this study demonstrated that immunization of mice with unadjuvanted and adjuvanted MTRV001 at 2 μg and 0.2 μg dose levels elicited robust anti-PLY IgG antibody titers. Unadjuvanted MTRV001, however, required 3 immunizations to elicit a high titer anti-PLY response and elicited lower anti-PLY titers than adjuvanted MTRV001 following both dose level regimens. For example, the MTRV001 2 μg dose level adjuvanted with aluminum hydroxide elicited antibody titers >10-fold higher than unadjuvanted MTRV001 at day 27 (two weeks following the second dose). The disparity in unadjuvanted versus adjuvanted MTRV001 induced titers was more pronounced at the 0.2 μg dose level where adjuvanted MTRV001 elicited anti-PLY IgG antibody titers >3 orders of magnitude higher than unadjuvanted MTRV001 at day 27. Anti-CbpA IgG antibody responses were generally low during the early stages of the dosing regimen (Day 0 to Day 27) before a rapid increase was observed two weeks following the third immunization (Day 41). As observed with the PLY antibody response, unadjuvanted MTRV001 required 3 immunizations to elicit a high-titer anti-CbpA antibody response and elicited lower anti-CbpA antibody titers than aluminum hydroxide adjuvanted MTRV001 at both dose levels. Collectively, these data demonstrate that aluminum hydroxide adjuvanted MTRV001 elicits higher titer anti-PLY and anti CbpA IgG antibody responses than unadjuvanted MTRV001 following a two-dose regimen at two different dose levels supporting the use of aluminum hydroxide for adjuvanting MTRV001 in a clinical study.

### Primary Pharmacodynamics Studies of MTRV001

Murine Preclinical Immunogenicity of MTRV001 and PLY-DM Followed by Intranasal Challenge with Clinical Isolates of S. pneumoniae Serotype 19F, 6B, and 22F and WT PLY Toxin Challenge

The MTRV001 precursor antigen, PLY-DM, was previously shown to be broadly protective against 17 of 20 different S. pneumoniae serotypes in a lethal intranasal (IN) murine challenge model (Thanawastien, 2021). To determine if MTRV001 immunization conferred protection, three murine studies (M227, M230, and M231) were performed to compare the immunogenicity and protective capacity of MTRV001 to PLY-DM against challenge with three S. pneumoniae serotypes.

In the initial protective efficacy study (M227), 30 female BALB/c mice were immunized intramuscularly (IM) once every two weeks for 3 injections with either 2 μg MTRV001, 2 μg of PLY DM, or phosphate buffered saline (PBS), each adjuvanted with 50 μg aluminum hydroxide (Alhydrogel®); for the other two studies (M230 and M231), 20 female BALB/c mice were immunized. Two weeks following the third immunization (Day 42), all mice in each group were bled, sera collected and pooled, and anti-PLY and anti-CbpA titers assessed by enzyme-linked immunosorbent assay (ELISA) (Table 7). In M227, 10 of the 30 mice from each group were used to assess protection against lethal WT PLY toxin challenge and the remaining 20 mice were used to assess protection against lethal IN challenge with two dose levels (10 mice/dose level) of a serotype 19F S. pneumoniae strain. For studies M230 and M231, all 20 mice were used to assess protection from lethal IN challenge using two dose levels of serotype 6B and 22F S. pneumoniae strains.

As shown in Table 7, mice immunized with MTRV001 and PLY-DM in each of the studies developed high and comparable levels of serum anti-PLY IgG antibodies that inhibited the in vitro hemolytic activity of WT PLY. In study M227, the anti-PLY response elicited from both MTRV001 and PLY DM conferred 100% protection against a lethal IV-administered dose of WT PLY toxin (100% survival of mice at 2 days post-toxin challenge with WT PLY), indicating that both antigens elicited protective levels of neutralizing anti-PLY antibodies in vivo. Conversely, the group of mice receiving PBS treatment showed 0% survival at 2 days post-toxin challenge with WT PLY. Anti-hemolytic titer was assayed from sera collected on Day 42 from one of the pools of sera from each group.

As expected, anti-CbpA antibodies were detected in sera from mice immunized with MTRV001 and titers were similar in all 3 studies. For M227, titer data is averaged from 3 pools of sera (10 mice/pool) while for M230 and M231, data is the average of 2 pools of sera (10 mice/pool). Surprisingly, anti-CbpA titers were detected in mice immunized with PLY-DM, although at much lower levels compared to MTRV001 immunized mice (ranging from 24-fold to 511-fold less). The reason for this is unclear, and in subsequent studies comparing MTRV001 and PLY-DM, immunization of mice with PLY-DM did not elicit a detectable anti-CbpA response.

TABLE 7
Survival, Anti-PLY and Anti-CbpA IgG Antibody Responses, and
Anti-Hemolytic Functional Antibody Titers Following Immunizations
with MTRV001 and PLY-DM and Intravenous Challenge with WT PLY

| | | | | | | S. |
|---|---|---|---|---|---|---|
| | | Day 42 | | | % | pneumoniae |
| | | Anti- | | Anti- | Survival | Challenge |
| Study | | CbpA | Anti-PLY | Hemolytic | WT PLY | Serotype |
| Number | Test Article | Titer ^{b} | Titer ^{b} | Titer ^{c} | challenge ^{(N=10) a} | (N = 20) |
| M227 | 2 μg | 37,333 | 320,000 | 150 | 100 | 19F |
| (N = 30 | MTRV001 | | | | | |
| mice/ | 2 μg PLY- | 73 | 640,000 | 300 | 100 | |
| group) ^{d} | DM | | | | | |
| | PBS | 10 | 10 | 75 | 0 | |
| M230 | 2 μg | 40,000 | 1,280,000 | 200 | NT | 6B |
| (N = 20 | MTRV001 | | | | | |
| mice/ | 2 μg PLY- | 1,640 | 1,280,000 | 200 | NT | |
| group) ^{d} | DM | | | | | |
| | PBS | 10 | 10 | 50 | NT | |
| M231 | 2 μg | 64,000 | 320,000 | 200 | NT | 22F |
| (N = 20 | MTRV001 | | | | | |
| mice/ | 2 μg PLY- | 480 | 240,000 | 150 | NT | |
| group) | DM | | | | | |
| | PBS | 10 | 10 | 50 | NT | |

^{a} Percent survival of mice at 2 days post-toxin challenge with WT PLY.
^{b} For M227, titer data is average from 3 pools of sera (10 mice/pool) while for M230 and M231, data is the average of 2 pools of sera (10 mice/pool).
^{c} Anti-hemolytic titer assayed from sera collected on Day 42 from one of the pools of sera from each group.
^{d} Groups of mice were immunized with 2 μg of antigen adjuvanted with 50 μg of Alhydrogel every two weeks for a total of 3 injections.
CbpA = choline binding protein A;
GMT = geometric mean titer;
IgG = immunoglobulin G;
PBS = phosphate buffered saline;
PLY-DM = pneumolysin double mutant;
WT PLY = wild-type pneumolysin.

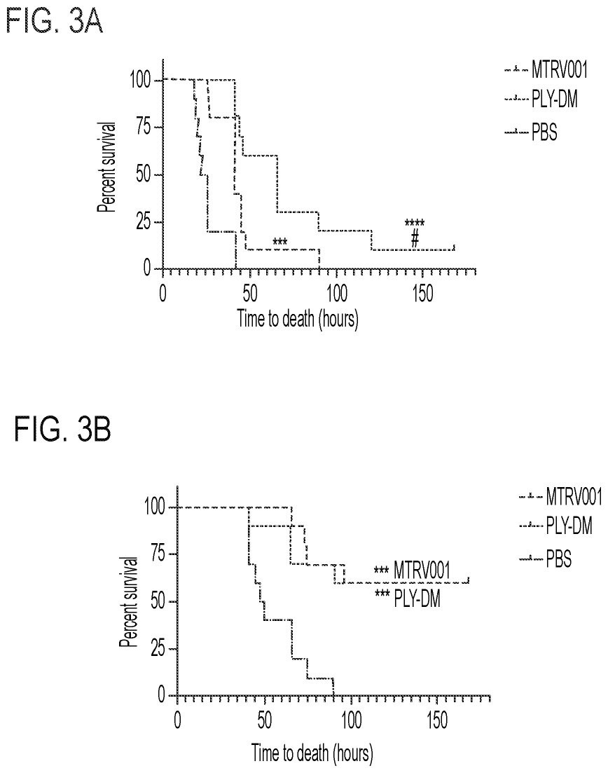

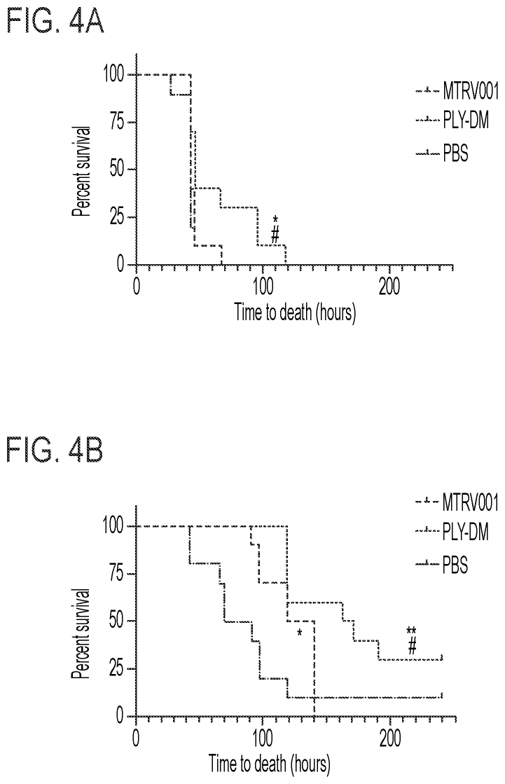

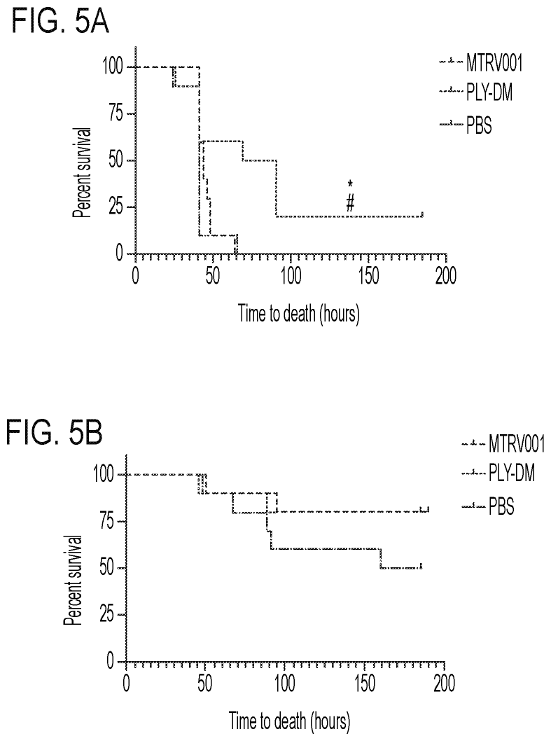

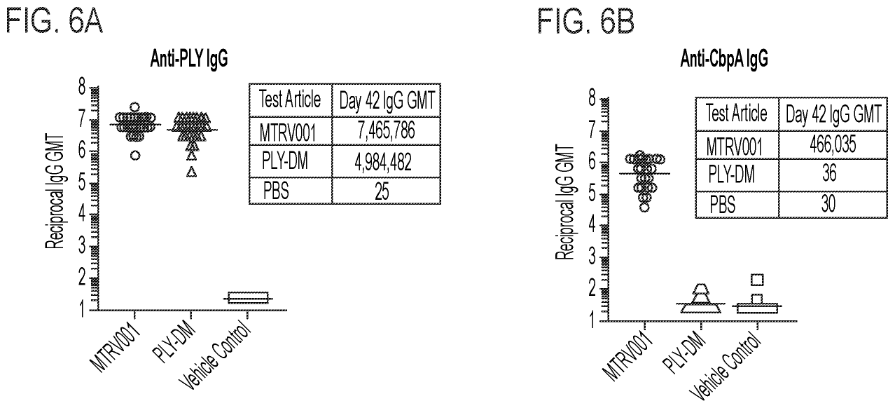

As shown in FIGS. 3-4, both MTRV001 and PLY-DM conferred statistically significant protection, prolonging time to death against the serotype 19F (FIGS. 3A-3B) and 6B (FIGS. 4A-4B) strains compared with the PBS control. Only PLY-DM conferred significant protection against the 22F strain at the high dose level compared to the PBS control (FIG. 5A), while both MTRV001 and PLY-DM immunized mice showed prolonged times to death at the lower infectious dose level (0.5×), although not statistically different from the PBS control (FIG. 5B). Despite MTRV001 and PLY DM eliciting similar anti-PLY titers (ELISA and functional), PLY-DM appeared to provide superior protection against bacterial challenge than MTRV001. However, because the number of mice challenged with each dose level of the S. pneumoniae strains was relatively low (N=10), further studies with higher numbers of mice would need to be performed to determine if the two antigens truly differ in their protective capacity.

Overall, MTRV001 elicits similar immunogenicity and protective immunity against WT PLY toxin challenge as PLY-DM. Furthermore, MTRV001 also provides significant protection against serotype 19F and 6B S. pneumoniae challenge.

Evaluation of MTRV001 and PLY-DM Efficacy following Intratracheal Infection with a Serotype 4 S. pneumoniae Strain

The efficacy contribution of the MTRV001 CbpA epitope(s) was evaluated in an animal model of S. pneumoniae disease. In this study, groups of mice were immunized with either adjuvanted MTRV001, adjuvanted PLY-DM, or a vehicle control and then challenged intratracheally (IT) with a lethal dose of a virulent serotype 4 S. pneumoniae strain (T4X). In addition to monitoring for survival, histopathology of heart, brain, and lung tissue was performed. A similar study was performed with YLN, which demonstrated that the CbpA epitope(s) significantly reduce lung pathology (Mann, 2014 and Table 6).

In the MTRV001 study, groups of 27 female BALB/c mice were immunized intraperitoneally (IP) every two weeks for a total of 3 injections with 10 μg of MTRV001, 10 μg of PLY-DM, or sterile saline (vehicle control), each adjuvanted with 130 μg of aluminum hydroxide (Alhydrogel®). Two weeks following the third immunization, all mice were bled, sera collected, and anti-PLY and anti-CbpA titers determined by ELISA. The mice were then infected IT with a lethal dose of the T4× S. pneumoniae strain. At 72 hours post-infection (hpi), 10 of the mice from each group were sacrificed, lungs removed and homogenized, and S. pneumoniae colony forming units (CFU)/mL determined. An additional 5 mice from each group were sacrificed and hearts, lungs, and brain removed for histopathologic analysis. The remaining mice were monitored for survival.

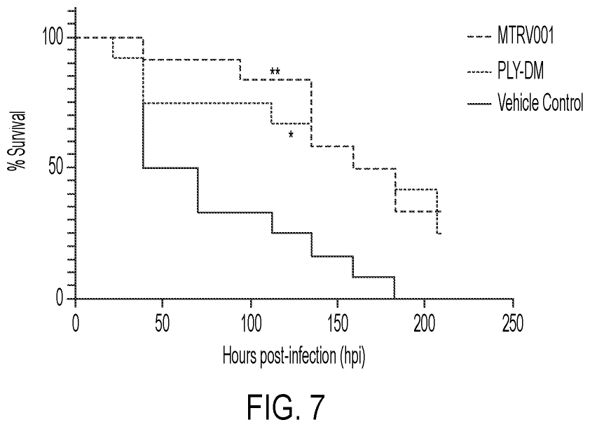

Individual mouse antibody ELISA titers at Day 42 (14 days following third immunization) as well as calculated GMT titers are shown in FIGS. 6A-6B. As shown in FIG. 6A, mice immunized with MTRV001 or PLY-DM developed robust anti-PLY IgG antibody responses compared to mice that received the vehicle control. As expected, only mice immunized with MTRV001 developed anti-CbpA titers, as shown in FIG. 6B. Immunization with both MTRV001 and PLY-DM conferred significant levels of protection to mice following challenge with S. pneumoniae strain T4X, compared to challenged vehicle control mice (**=p-value <0.01; *=p-value <0.1; compared to vehicle control). As shown in FIG. 7, although MTRV001 immunized mice demonstrated improved survival at early timepoints post-infection compared to mice immunized with PLY-DM (0-125 hours post-infection), by the end of the monitoring period no significant difference between the two groups of mice was observed.

As shown in Table 8, evaluation of bacterial dissemination to the lungs following the challenge indicated that MTRV001 and PLY-DM immunized mice had fewer T4× colony forming units (CFU) in the lungs at 72 hours post-infection compared to vehicle controls; however, only the CFU in the lungs of PLY DM immunized were significantly lower than the saline control group (*p<0.0001 by unpaired student T test). PLY-DM=pneumolysin double mutant; CFU=colony forming unit; SD=standard deviation.

TABLE 8
S. pneumoniae CFU in Mouse Lungs 72 Hours Post-Infection
with Virulent Serotype 4 S. pneumoniae Strain

| Treatment | Mean log₁₀ CFU/lung (N = 10) | SD |
|---|---|---|
| MTRV001 | 3.76 | 0.87 |
| PLY-DM | 2.86* | 0.59 |
| Vehicle control | 4.14 | 0.67 |

*p < 0.0001 by unpaired student T-test.
PLY-DM = pneumolysin double mutant; CFU = colony forming unit; SD = standard deviation.

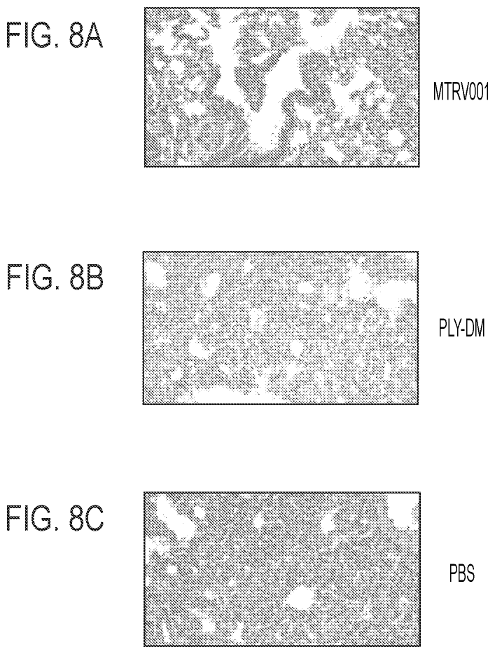

Histopathology was performed on lung, heart, and brain tissue from immunized and unimmunized mice following T4× challenge. No microscopic abnormalities were observed in the heart and brains of any animals. However, as shown in FIGS. 8A-8C, the lungs from unimmunized and PLY-DM immunized mice exhibited mixed immune cell inflammation of the alveoli and interstitium, indicating pathology in 4 of 5 animals per group. In sharp contrast, all five mice administered MTRV001 exhibited normal lung architecture with no evidence of lung inflammation of alveoli or interstitium (FIG. 8A).

Collectively, these studies indicate that whereas immunization of mice with either MTRV001 or PLY-DM conferred similar levels of protection against death, only MTRV001 conferred protection from lung inflammation and pathology, demonstrating an important role of the MTRV001 CbpA epitope(s) in eliciting protective antibodies.

### Dose-Immunogenicity Study of Alhydrogel®-AdjuvantedMTRV001

Next, the impact of the Alhydrogel® adjuvant on the MTRV001 anti-PLY and anti-CbpA antibody response as a function of the onset of antibody induction, the overall antibody response following all immunizations, and the durability of the antibody response were examined.

Groups of 5 female BALB/c mice were immunized IM every two weeks for three injections (Day 0, 14, and 28) with 2 μg or 0.2 μg MTRV001 either unadjuvanted or adjuvanted with 50 μg Alhydrogel®. Sterile saline without adjuvant was administered to a group of mice as a control. Mice were bled on Day 0, 6, 13, 27, 41, 70, and 98, sera were collected, and anti-PLY and anti-CbpA titers determined by ELISA. At Day 0, 6, 13, and 27 individual mouse antibody titers were determined, and a GMT calculated. At Day 41, Day 70, and Day 98, collected sera were pooled and a group titer determined.

At the 2 μg MTRV001 dose level, unadjuvanted and adjuvanted MTRV001 elicited robust anti-PLY IgG antibody responses that were similar in magnitude following 3 immunizations (Day 41) and comparably durable at Day 98 (Table 9). However, the adjuvanted 2 μg MTRV001 dose level elicited a more rapid rise in anti-PLY response than the 2 μg unadjuvanted dose level, with a 30-fold higher titer observed two weeks after the second immunization (Day 27). At the 0.2 μg MTRV001 dose level, the adjuvanted MTRV001 elicited both a faster induction of anti-PLY response and a greater magnitude response by Day 41 than the unadjuvanted MTRV001. Both adjuvanted and unadjuvanted 0.2 μg MTRV001 elicited a durable anti-PLY response out to 98 days.

TABLE 9:
Anti-PLY IgG Serum GMT and Pooled Titers Following IM
Immunization of BALB/c Mice with MTRV001 ± Adjuvant

| | Anti-PLY GMT (N = 5) | | | | Pooled Sera Titers (from N = 5) | | |
|---|---|---|---|---|---|---|---|
| Test Article | Day 0 | Day 6 | Day 13 | Day 27 | Day 41 | Day 70 | Day 98 |
| 2 μg MTRV001 | 10 | 11 | 10 | 13,929 | 800,000 | 800,000 | 1,600,000 |
| 0.2 μg MTRV001 | 10 | 10 | 10 | 277 | 192,000 | 320,000 | 640,000 |
| 2 μg MTRV001 + | 10 | 630 | 27,858 | 422,243 | 1,600,000 | 1,600,000 | 3,200,000 |
| adjuvant | | | | | | | |
| 0.2 μg MTRV001 + | 10 | 20 | 6,370 | 175,770 | 1,600,000 | 1,600,000 | 3,200,000 |
| adjuvant | | | | | | | |
| Sterile saline | 10 | 10 | 10 | 10 | 10 | 10 | 10 |

GMT = geometric mean titer;
IgG = immunoglobulin G;
IM = intramuscular(ly).

As shown in Table 10, a similar anti-CbpA response profile was elicited by the adjuvanted and unadjuvanted MTRV001 at the 2 μg dose level at Day 41 that was durable out to 98 days. However, like the anti-PLY response, a higher anti-CbpA titer was observed at Day 27 following administration of adjuvanted MTRV001 compared to unadjuvanted MTRV001. Similarly, the adjuvanted 0.2 μg MTRV001 dose level regimen elicited higher anti-CbpA titers at early timepoints compared to the unadjuvanted dose regimen. However, by Day 41, similar titers were observed between the adjuvanted and unadjuvanted 0.2 μg MTRV001 dose levels. The anti-CbpA antibody response with adjuvanted 0.2 μg MTRV001 increased at Day 70 and Day 98 while a decrease in titers was observed with the unadjuvanted MTRV001, indicating the presence of adjuvant at this dose level promoted a more durable anti-CbpA antibody response.

TABLE 10
Anti-CbpA IgG Serum GMT and Pooled Serum Titers following IM
Immunization of BALB/c Mice with MTRV001 ± Adjuvant

| | Anti-CbpA GMT (N = 5) | | | | Pooled Sera Titers (from N = 5) | | |
|---|---|---|---|---|---|---|---|
| Test Article | Day 0 | Day 6 | Day 13 | Day 27 | Day 41 | Day 70 | Day 98 |
| 2 μg MTRV001 | 10 | 10 | 10 | 61 | 32,000 | 80,000 | 64,000 |
| 0.2 μg MTRV001 | 10 | 10 | 10 | 17 | 64,000 | 16,000 | 16,000 |
| 2 μg MTRV001 + | 10 | 13 | 30 | 420 | 12,800 | 40,000 | 32,000 |
| adjuvant | | | | | | | |
| 0.2 μg MTRV001 + | 10 | 10 | 10 | 192 | 32,000 | 80,000 | 64,000 |
| adjuvant | | | | | | | |
| Sterile saline | 10 | 10 | 10 | 10 | 10 | 10 | 10 |

GMT = geometric mean titer;
IgG = immunoglobulin G;
IM = intramuscular(ly).

These data demonstrate that although adjuvant does not enhance the magnitude of the anti-PLY and anti-CbpA titers following three immunizations of a high dose of MTRV001 (2 μg), it does engender a more rapid induction of antibody response. Furthermore, at a 10-fold lower dose level of MTRV001 (0.2 μg), adjuvant enhanced the magnitude of the anti-PLY antibody response as well as improved the durability of the anti-CbpA antibody response. Overall, the data support the use of aluminum hydroxide as an adjuvant to augment the immunogenicity of MTRV001.

### In Vivo Immunogenicity of MTRV001 in New Zealand White Rabbits

The immunogenicity of MTRV001, the desired effect, and the potential toxicological impact of the immune response was assessed in a GLP repeat-dose toxicity study in rabbits. [0309]5 dose groups with 10 rabbits/sex/group administered 10 μg, 30 μg, and 90 μg MTRV001 adjuvanted with aluminum hydroxide, 90 μg MTRV001 unadjuvanted, or the adjuvanted vehicle control. Rabbits were dosed via intramuscular (IM) injection on days 1, 15, and 29, using a constant dosing volume of 0.5 mL. Blood samples for antibody determination were collected pre-test, prior to dosing on days 15 and 29, and prior to scheduled necropsies on days 31 and 43. Serum anti-PLY IgG and anti-CbpA titers for individual rabbits were determined by PLY- and CbpA-ELISAs and GMT calculated for the group. Individual rabbit antibody responses were compared to baseline serum collected from all rabbits prior to administration of the first dose at each dose level (Day −5) to determine seroconversion (defined as an antibody response ≥5-fold over pre-immunization levels)

TABLE 11
Anti-PLY IgG Antibody Geometric Mean Titer and Seroconversion

| | | | | Anti-CbpA IgG Geometric Mean Titer | | | |
|---|---|---|---|---|---|---|---|
| | | Target Dose | | (Percent Seroconversion) ^{a} | | | |
| | | MTRV001 | Adjuvant | Day −5 | Day 15 | Day 29 | Day 43 |
| Group | Treatment | (μg) | (mg) | (N = 20) ^{b} | (N = 20) ^{b} | (N = 20) ^{b} | (N = 10) ^{c} |
| 1 | Vehicle control + | 0 | 0.5 | 25 | 31 | 27 | 84* |
| | adjuvant | | | | (5) | (0) | (22) |
| 2 | Low dose | 10 | 0.5 | 16 | 664 | 30,901* | 32,000*^{#} |
| | vaccine + adjuvant | | | | (90) | (100) | (100) |
| 3 | Mid dose | 30 | 0.5 | 25 | 2,471 | 68,431* | 48,503*^{#} |
| | vaccine + adjuvant | | | | (95) | (100) | (100) |
| 4 | High dose | 90 | 0 | 26 | 2,909 | 76,019* | 90,510*^{A} |
| | vaccine only | | | | (90) | (100) | (100) |
| 5 | High dose | 90 | 0.5 | 21 | 2,965 | 119,145*† | 103,968*^{A} |
| | vaccine + | | | | (95) | (100) | (100) |
| | adjuvant | | | | | | |

^{a} Percent seroconversion defined as anti-PLY titer ≥ 10-fold over pre-immune titer.
^{b} Days −5 (pre-immune), 15, and 29 (post-immunization) serum antibody GMTs assayed from all 20 rabbits per group.
^{c} Day 43 GMT assayed for the 10 remaining rabbits per group designated for recovery necropsy.
*p < 0.05, compared to Day −5 pre-immune, Dunnett's.
†p < 0.05, compared to unadjuvanted 90 μg MVX0I at Day 29, Tukey's multiple comparisons.
^{#}p < 0.05, compared to vehicle control at Day 43, Tukey's multiple comparisons.
^{A}p < 0.05, compared to 10 and 30 μg MTRV001 and vehicle control at Day 43, Tukey's multiple comparisons.
IgG: immunoglobulin G;
GMT: geometric mean titer;
PLY: pneumolysin.

As shown in Table 11, MTRV001 elicited a dose-dependent IgG antibody response against PLY. Days 5 (pre-immune), 15, and 29 (post-immunization) serum antibody GMTs were assayed from all 20 rabbits per group. Day 43 GMT was assayed for the 10 remaining rabbits per group designated for recovery necropsy. There was a statistically significant increase in anti-PLY GMTs on days 15, 29, and 43 in rabbits that were administered any dose level of adjuvanted MTRV001 (10 g, 30 g, and 90 g) compared to pre-immunization controls. Rabbits immunized with adjuvanted dose levels of MTRV001 developed anti-PLY GMTs in a dose-dependent fashion ranging from 2,000 to 4,950-fold above baseline (10 and 90 μg dose levels, respectively). The unadjuvanted 90 μg MTRV001 dose level elicited a ˜3,500-fold increase in GMT above baseline at day 43 (2 weeks after the third and final immunization). The unadjuvanted 90 μg MTRV001 dose regimen also elicited a significantly higher anti-PLY titer over time compared to pre-immune controls. Seroconversion against PLY was observed for 90% to 95% of rabbits, at all dose levels, at 14 days after the first immunization (day 15). Fourteen days after the second vaccination (day 29), 100% of rabbits seroconverted for all adjuvanted and unadjuvanted dose levels, and this seroconversion level was maintained to day 43 (study termination). Percent seroconversion is defined as anti-PLY titer ≥10-fold over pre-immune titer. *=p<0.05, compared to Day −5 pre-immune, Dunnett's. †=p<0.05, compared to unadjuvanted 90 μg MVX0I at Day 29, Tukey's multiple comparisons. ^{#}=p<0.05, compared to vehicle control at Day 43, Tukey's multiple comparisons. ^{A}=p<0.05, compared to 10 and 30 μg MTRV001 and vehicle control at Day 43, Tukey's multiple comparison. As shown in Table 12, the CbpA peptides also elicited CbpA-specific titers in rabbits immunized with the adjuvanted MTRV001. Overall, the anti-CbpA titers were much lower than those observed for anti-PLY and was confounded by a high background as evidenced by the higher anti-CbpA titer at day 43 in the vehicle control group relative to the pre-immunization sera. Three adjuvanted MTRV001 immunizations were required to elicit significant levels of anti-CbpA antibodies above pre-immune titers. At day 43, only animals immunized at the 30 μg MTRV001 dose level elicited a significantly higher anti-CbpA GMT relative to the vehicle control. The lower anti-CbpA titers were also reflected in the rate of CbpA-specific seroconversion. The higher background (or more non-specific response) in rabbits against CbpA was reflected in a 30% seroconversion rate of rabbits immunized with vehicle control at day 43 (study termination). The CbpA-specific percent seroconversion increased after each administration for rabbits immunized with adjuvanted MTRV001 at all dose levels. The highest seroconversion rate for CbpA-specific responses induced by MTRV001 (70%) was observed at the 30 μg dose level following the third immunization (days 43).

TABLE 12
Anti-CbpA IgG Antibody Geometric Mean Titer and Seroconversion

| | | | | Anti-CbpA IgG Geometric Mean Titer | | | |
|---|---|---|---|---|---|---|---|
| | | Target Dose | | (Percent Seroconversion) ^{a} | | | |
| | | MTRV001 | Adjuvant | Day −5 | Day 15 | Day 29 | Day 43 |
| Group | Treatment | (μg) | (mg) | (N = 20) ^{b} | (N = 20) ^{b} | (N = 20) ^{b} | (N = 10) ^{c} |
| 1 | Vehicle | 0 | 0.5 | 32 | 29 | 36 | 130* |
| | control + | | | | (0) | (5) | (30) |
| | adjuvant | | | | | | |
| 2 | Low dose | 10 | 0.5 | 83 | 110 | 121 | 393* |
| | vaccine + | | | | (10) | (25) | (40) |
| | adjuvant | | | | | | |
| 3 | Mid dose | 30 | 0.5 | 33 | 75 | 146 | 495*† |
| | vaccine + | | | | (21) | (42) | (70) |
| | adjuvant | | | | | | |
| 4 | High dose | 90 | 0 | 71 | 79 | 275* | 243 |
| | vaccine only | | | | (5) | (30) | (30) |
| 5 | High dose | 90 | 0.5 | 94 | 49 | 197 | 544* |
| | vaccine + | | | | (0) | (21) | (40) |
| | adjuvant | | | | | | |

^{a} Percent seroconversion defined as anti-CbpA titer ≥ 5-fold over background (pre-immune titer)
^{b} Days −5 (pre-immune), 15, and 29 (post-immunization) serum antibody GMTs assayed from all 20 rabbits per group.
^{c} Day 43 GMT assayed for the 10 remaining rabbits per group designated for recovery necropsy.
*p < 0.05, compared to Day −5 pre-immune, Dunnett's.
†p < 0.05, compared to unadjuvanted 90 μg MVX0I at Day 29, Tukey's multiple comparisons.
CbpA = choline binding protein A;
IgG = immunoglobulin G;
GMT = geometric mean titer.

[symbol]

anti-CbpA IgG antibody response in rabbits, in a dose-dependent fashion, following the three dose immunization regimens.

### Primary Pharmacodynamics Studies of PLY-DM

PLY-DM is an antigen that preceded MTRV001 in development and lacks the CbpA moieties of MTRV001. The PLY-DM genetic toxoid contains two amino acid substitutions (G293S and L460D) that render the antigen nontoxic (undetectable levels of cytolytic activity) and is the PLY moiety of MTRV001, as depicted in the schematic in FIG. 1B.

### In Vivo Immunogenicity and Efficacy of PLY-DM in Naïve Mice

A series of 14 nonclinical studies were conducted to evaluate whether mice actively immunized with PLY-DM were protected from lethal IN challenge with a variety of S. pneumoniae strains and serotypes (Thanawastien, 2021). Collectively, 28 different S. pneumoniae strains, representing 20 different serotypes from various parts of the world and isolated from different body sites (bacteremia, pneumonia, carriage), with differing degrees of virulence, were evaluated. The 20 serotypes include those covered by Prevnar 13® as well as selected emerging serotypes.

In each of the studies, groups of 20 naive BALB/c mice were immunized IM with 2 g PLY-DM adjuvanted with 50 μg of aluminum hydroxide (Alhydrogel®) or a PBS control every two weeks for 3 injections. Two weeks following the third immunization, mice were bled, sera collected and pooled, and serum anti-PLY antibodies levels determined by ELISA. The mice were then infected IN with 2 dose levels (10 mice/dose level) of various virulent S. pneumoniae strains. Challenge dose levels were determined from the preliminary 50% of the lethal dose (LD50) studies. The first challenge dose (1×) was the fewest number of bacteria that were 100% lethal in the LD50 study and the second dose (0.5×) of bacteria was half the lethal dose.

With nearly every S. pneumoniae strain, except the serotype 23B strain, the 1× dose level resulted in the death of most of the mice in the unimmunized group. The survival curves of the immunized and unimmunized groups were compared using a log-rank Mantel-Cox test. The cumulative results from these studies demonstrate that active immunization of mice with PLY-DM conferred statistically significant protection against 22 (79%) of the 28 strains tested and 17 (85%) of the 20 representative serotypes evaluated (Table 13 and Thanawastien, 2021). For some of the serotypes, multiple strains were evaluated and for some, differing levels of protection were observed between the strains. For serotype 22F, significant protection was only observed for two of the three strains evaluated; however, because PLY-DM elicited protective was not considered a protected serotype since only one of the four serotype 3 strains evaluated showed a significant improvement in time to death. Both serotype 1 and 3 strains displayed a high level of virulence in this model with infectious dose levels that were 5 to 100-fold lower than used for other serotypes and 100% lethality was observed at even the 0.5× dose level. While it could not be claimed that immunization with PLY-DM provided significant protection against serotype 1 and 3 based on the results, it is possible that a larger difference in survival or time to death would have been observed at lower challenge dose levels. Serotype 23B strain was not infectious in this mouse model and was not included in the protected serotypes. It is possible that immunization with PLY-DM would be protective against this serotype with a more virulent 23B strain.

Overall, immunization with PLY-DM demonstrated broad, non-serotype dependent protection of mice from lethal IN S. pneumoniae infection. Moreover, it was observed that PLY-DM immunized mice were protected from lethal infection regardless of whether the strains were isolated from different geographical locations, different body locations (i.e., nasopharynx, blood, sputum), or different disease indications (i.e., pneumonia, bacteremia).

TABLE 13
Anti-PLY IgG Antibody Titers and Survival Results of Mice Immunized with
PLY-DM or PBS Following Challenge with Virulent S. pneumoniae Strains

| | | Percent Mouse Survival - Time to Death (Hours Post-Infection) | | | | | | | |
|---|---|---|---|---|---|---|---|---|---|
| | | 1 × S. pneumoniae Infection | | | | 0.5 × S. pneumoniae Infection | | | |
| | Day 42 | Dose ^{c} | | | | Dose ^{c} | | | |
| | Anti-PLY | PLY-DM | | | | PLY-DM | | | |
| Serotype ^{a} | IgG Titer ^{b} | Immunized | | PBS | | Immunized | | PBS | |
| 1 | 1,280,000 | 0 | (51) | 0 | (47) | 0 | (53) | 0 | (52) |
| 3 | 1,026,043 | 0 | (42)* | 0 | (39) | 0 | (54)† | 0 | (42) |
| | 1,280,000 | 0 | (45) | 0 | (40) | 0 | (62) | 0 | (64) |
| | 3,200,000 | 0 | (58) | 0 | (57) | 0 | (67) | 0 | (63) |
| | 2,560,000 | 0 | (62) | 10 | (66) | 0 | (79) | 10 | (86) |
| 5 | 1,280,000 | 100 | (240)^{A} | 0 | (71) | 100 | (240) | 100 | (240) |
| 6A | 565,386 | 0 | (58)* | 0 | (39) | 100 | (142)^{A} | 0 | (50) |
| 6B | 1,280,000 | 0 | (35)^{A} | 0 | (20) | 60 | (166)^{A} | 0 | (36) |
| 8 | 2,560,000 | 0 | (46)* | 0 | (43) | 0 | (57) | 0 | (50) |
| | 720,000 | 20 | (54)^{A} | 0 | (17) | 90 | (149)† | 30 | (68) |
| 11A | 985,092 | 0 | (30)† | 0 | (22) | 90 | (154)^{#} | 10 | (49) |
| 14 | 836,807 | 100 | (168)^{A} | 10 | (81) | 100 | (168) | 100 | (168) |
| 15A | 1,200,000 | 40 | (153)^{A} | 0 | (19) | 100 | (240)^{A} | 0 | (51) |
| 15B/C | 723,598 | 80 | (198)^{A} | 0 | (20) | 100 | (264)† | 30 | (78) |
| 18C | 721,406 | 90 | (128)^{A} | 0 | (26) | NA | | NA | |
| 19F | 627,830 | 10 | (43) | 10 | (40) | 100 | (216)† | 30 | (82) |
| | 2,560,000 | 0 | (31)^{#} | 0 | (19) | 20 | (73)^{#} | 0 | (31) |
| | 3,200,000 | 20 | (77)^{#} | 0 | (24) | 70 | (144)† | 20 | (55) |
| 19A | 507,230 | 10 | (58)^{A} | 0 | (18) | 100 | (168)† | 40 | (70) |
| 22F | 866,050 | 0 | (45) | 0 | (52) | 80 | (222) | 70 | (218) |
| | 1,280,000 | 40 | (95)^{A} | 0 | (23) | 90 | (227)^{#} | 17 | (54) |
| | 1,200,000 | 78 | (132)† | 10 | (60) | 100 | (168) | 75 | (121) |
| 23F | 600,000 | 50 | (110)* | 10 | (55) | 100 | (168) | 100 | (168) |
| 23A | 960,000 | 90 | (157)* | 30 | (105) | 100 | (168) | 90 | (162) |
| 23B | 1,200,000 | 100 | (168) | 90 | (144) | 100 | (168) | 100 | (168) |
| 33F | 760,490 | 0 | (47)^{#} | 0 | (25) | 100 | (168)† | 40 | (96) |
| 35B | 1,280,000 | 22 | (82)† | 0 | (30) | 90 | (148) | 100 | (168) |

*p-value 0.05-0.01
†p-value 0.01-0.001
^{#}p-value 0.001-0.0001
^{A}p-value < 0.0001 Log-rank Mantel-Cox test
^{a} S. pneumoniae challenge serotype
^{b} Anti-PLY antibody titers induced by IM administration of PLY-DM at a biweekly interval (Days 0, 14, and 28). Sera was assayed by ELISA at Day 42, 2 weeks following the third and final immunization. The anti-PLY IgG titer was determined by using endpoint titer cut-off method.
^{c} Challenge dose of clinical isolates were determined by LD50. 1 × dose was the highest dose from LD50 study that caused near 100% lethality. The 0.5 × dose was a 1:1 dilution of the 1 × dose.
ELISA: enzyme-linked immunosorbent assay; IgG: immunoglobulin G; IM: intramuscular(ly); LD50: lethal dose, 50%; PBS: phosphate-buffered saline; PLY: pneumolysin; PLY-DM: double-mutant pneumolysin.

### Supportive Studies of the MTRV001 Related Antigen YLN

Data from nonclinical studies performed with YLN, a MTRV001 precursor antigen that differs in sequence by a single amino acid in the PLY moiety, have been previously published (Mann, 2014). The findings from these studies are summarized below.

Studies Evaluating the MTRV001 Variant YLN (PLY-SM/CbpA Fusion Protein) and its Components (Mann, 2014) [symbol]
fragments as MTRV001 fused to the N- and C-termini of a PLY genetic toxoid that harbors a single amino acid substitution L460D (FIG. 1B, “PLY-SM”). YLN was constructed and evaluated for immunogenicity and efficacy relative to PLY-SM. In this evaluation, YLN and PLY-SM adjuvanted with aluminum hydroxide and administered IP every two weeks for three injections at a 10 μg dose level elicited similar anti-PLY titers that inhibited the in vitro hemolytic activity of WT PLY. YLN also elicited a robust anti-CbpA antibody response that was greater than that observed with a truncated form of CbpA (CbpA R2). Following immunization, mice were challenged IT with a serotype 4 S. pneumoniae strain and evaluated for survival, presence of bacteria in cerebrospinal fluid (CSF) (meningitis), and lung pathology. A separate group of immunized mice were challenged IN with a serotype 19F strain and assessed for presence of bacteria in the nasopharynx (colonization) and ear (otitis media).

Although no difference in survival was observed following IT challenge between mice immunized with YLN and PLY-SM (Mann, 2014; FIG. 5A), significantly fewer mice immunized with YLN had detectable bacteria in the CSF (Mann, 2014; FIG. 5E). Furthermore, mice immunized with YLN exhibited normal lung architecture following infection whereas unimmunized mice and mice immunized with PLY-SM demonstrated overt signs of pathology including inflammation, immune cell infiltration, and hemorrhage (Mann, 2014; FIG. 5D). YLN immunized mice challenged IN with the 19F strain demonstrated significantly less bacterial load in the ears relative to mice immunized with PLY-SM (Mann, 2014; FIG. 5C) and in the nasopharynx at Day 7 post-infection (Mann, 2014; FIG. 5B). Collectively, these data demonstrate that the addition of the CbpA peptide(s) to the PLY-SM toxoid did not impact elicitation of an anti-PLY antibody response and conferred additional protection against bacterial invasion of CSF, the ear, and lung pathology as well as showing some reduction in the density of colonization of the nasopharynx.

### MTRV001 In Vivo Studies to Develop an Immunization Protocol for PLY and CbpA Immunopotency Release and Stability Analyses

Studies were conducted to define the optimal immunization regimen and MTRV001 dose level for evaluating the in vivo PLY and CbpA immunopotency of MTRV001 for routine quality control testing of MTRV001 drug substance and drug product.

In the primary pharmacology studies evaluating MTRV001 efficacy, the dosing regimen consisted of three injections administered once every two weeks (Day 0, 14, and 28). Given that sera are not collected until 14 days following the last immunization and the time required for ELISA testing of the sera, investigations were conducted to determine if a suitable obtain results. For the immunization protocol to support the quality control testing, the dosing regimen was changed to once weekly for 3 injections based on a study demonstrating that ≥2 μg MTRV001 dose levels elicited high titer antibodies to both PLY and CbpA. The PLY and CbpA titers were lower than observed with the regimen of every other week dosing but were deemed sufficient for further optimization to define the immunization protocol for quality control testing. The nonclinical studies performed to optimize the immunization protocol are described in this section.

### Dose-Ranging Study of In Vivo Immunogenicity and Efficacy of MTRV001 in Naïve Mice

The purpose of this dose-ranging study was to evaluate the immunogenicity and efficacy of MTRV001 after weekly administration for 2 or 3 injections to determine if the immunization schedule could be shortened further from the 3-dose weekly regimen.

In this study, groups of naïve BALB/c mice (10 females per group) were immunized IM 2 times (Days 0 and 7) or 3 times (Day 0, 7, and 14) with 3 different dose levels (0.5, 3, or 5 μg) of MTRV001 adjuvanted prior to administration (as would be done for drug substance quality control testing), with 3 μg of MTRV001 adjuvanted at the time of manufacture (representative of drug product samples), or with buffer (PBS) control. All test articles and PBS control contained 50 μg aluminum hydroxide (Alhydrogel®) adjuvant. Two weeks after the final immunization, mice were bled, sera collected, and the anti-PLY and anti-CbpA titer determined by ELISA. For the 2-dose regimen (Table 14), sera were pooled and a titer determined for the group; however, for the 3-dose regimen (Table 15), the titer was determined from individual mice and a GMT calculated for the group. The serum samples from the 3-dose regimen were also assayed for toxin-neutralizing, or functional, antibody responses using an in vitro anti-hemolysis assay. The mice in each group (2- and 3-dose regimen) were challenged IV with a lethal dose of WT PLY toxin (1.5 μg) and monitored for survival to evaluate the protective immune response from the MTRV001 dose levels/regimens.

Pooled serum anti-PLY IgG titers remained consistent between the MTRV001 dose levels adjuvanted prior to administration (drug substance) and were also consistent with the pre adjuvanted (drug product) MTRV001 (Table 14). Two weeks after the second and final immunization, the pooled serum anti-CbpA titer elicited by MTRV001 drug substance increased from the 0.5 μg dose to the 3 μg dose but then decreased slightly with the 5 μg dose. The 3 μg dose of MTRV001 drug product elicited a similar titer to the equivalent dose of drug substance. Following the MTRV001 2-dose weekly regimen, the percent of survival of mice (5 μg drug substance) and 78% (3 μg drug substance), as shown in Table 14. All mice administered a MTRV001 immunization regimen showed a significant increase in survival and time to death compared to the PBS control group. However, the survival rate was not statistically significant between MTRV001 immunized mice at any dose level administered in the 2-dose weekly regimen.

TABLE 14
2-Dose Weekly Regimen: Anti-CbpA and Anti-PLY IgG
Titers and Survival following WT PLY Challenge

| | | Anti-PLY Response | | | |
|---|---|---|---|---|---|
| | Anti-CbpA | Day 23 | WT PLY Challenge | | |
| | Response | Anti- | | | Geometric |
| | Day 23 Anti- | PLY IgG | Number | % Survival | mean TTD |
| Test Article | CbpA IgG Titer | Titer | of Mice | (1440 mpc) | (minutes) |
| 3 μg MTRV001 | 6,400 | 80,000 | 9 | 67% *** | 395.7 |
| DP | | | | | |
| 0.5 μg MTRV001 | 800 | 80,000 | 10 | 60% **** | 304.3 |
| DS | | | | | |
| 3 μg MTRV001 | 8,000 | 160,000 | 9 | 78% **** | 656.7 |
| DS | | | | | |
| 5 μg MTRV001 | 3,200 | 80,000 | 10 | 50% **** | 95.4 |
| DS | | | | | |
| PBS Control | 10 | 10 | 10 | 0% | 2.0 |

*** p value of < 0.001 when compared with PBS; log-rank Mantel-Cox Test
**** p value of < 0.0001 when compared with PBS; log-rank Mantel-Cox Test
CbpA = choline binding protein A; DS = drug substance; DP = drug product; IgG = immunoglobulin G; mpc = minutes post-challenge; PBS = phosphate buffered saline; PLY = pneumolysin; TTD = time to death.

Following the MTRV001 3-dose immunization regimen, sera from mice administered any MTRV001 test article exhibited high anti-PLY and anti-CbpA titers, as shown in Table 15. No significant differences were observed in anti-PLY and anti-CbpA titers between the drug substance dose levels; however, the titers were similar between drug substance and drug product samples at the same dose level. The high anti-PLY IgG titers correlated with an ability of the sera to inhibit the cytolytic activity of WT PLY in an in vitro hemolytic assay (i.e., more functional antibody). All of the mice immunized with MTRV001 test articles survived IV challenge with a lethal dose of WT PLY toxin, indicating the immune response was sufficient at all dose levels to neutralize toxin activity.

Given that the 3-dose weekly regimen of MTRV001 conferred superior protection to mice compared to the 2-dose regimen, subsequent immunopotency studies were performed elicit a dose related effect, an additional dose-ranging study evaluating dose levels below 0.5 μg MTRV001 was performed.

TABLE 15
3-Dose Weekly Regimen: Anti-CbpA Anti-PLY IgG Titers, Anti-
Hemolytic Titers, and Survival following WT PLY Challenge

| | Anti-CbpA | Anti-PLY Response | | | | |
|---|---|---|---|---|---|---|
| | Response | Day 28 | Day 28 | WT PLY Challenge | | |
| | Day 28 | Anti- | Anti- | | % | Geometric |
| | Anti-CbpA | PLY IgG | hemolytic | Number | Survival | mean TTD |
| Test Article | IgG Titer | Titer | Titer | of Mice | (1440 mpc) | (minutes) |
| 3 μg MTRV001 | 15,647 | 393,966 | 150 | 10 | 100% | 1440.0 |
| DP | | | | | | |
| 0.5 μg MTRV001 | 16,969 | 211,121 | 75 | 10 | 100% | 1440.0 |
| DS | | | | | | |
| 3 μg MTRV001 | 15,935 | 393,966 | 150 | 10 | 100% | 1440.0 |
| DS | | | | | | |
| 5 μg MTRV001 | 14,446 | 226,274 | 150 | 10 | 100% | 1440.0 |
| DS | | | | | | |
| PBS Control | 14 | 10 | NT | 10 | 0% | 3.8 |

CbpA = choline binding protein A;
DS = drug substance;
DP = drug product;
IgG = immunoglobulin G;
mpc = minutes post-challenge;
NT = not tested;
PBS = phosphate buffered saline;
PLY = pneumolysin;
TTD = time to death.

### Further Dose-Ranging Study of In Vivo Immunogenicity and Efficacy of MTRV001 in Naïve Mice

The initial dose-ranging study indicated that anti-PLY and anti-CbpA titers in mice remained robust and comparable at MTRV001 dose levels between 0.5 and 5 μg administered once weekly for 3 injections. Since the effective dose level was not determined in that study, this study was performed using dose levels less than 0.5 μg MTRV001 to determine the effective dose level for the quality control testing immunization procedure.

Groups of 5 female BALB/c mice were immunized IM once weekly for 3 injections (Days 0, 7, and 14) with 0.01, 0.03, 0.1, 0.3, and 3 μg dose levels of MTRV001 (pre-adjuvanted at time of manufacture) or PBS containing 1 mg/mL aluminum hydroxide. The MTRV001 doses were prepared from a lot of MTRV001 that contained 60 μg/mL of MTRV001 and 1 mg/mL aluminum hydroxide using sterile saline as the diluent. Therefore, as the dose level decreased, the level of aluminum hydroxide decreased. Fourteen days after the third immunization, mice were bled, sera collected and pooled, and anti-PLY and anti-CbpA titers were assayed by ELISA. The sera were also analyzed for the induced functional anti-PLY titer red blood cells by WT PLY. In addition, immunized mice were evaluated for protection against IV challenge with a lethal dose of WT PLY toxin.

Dose-dependent anti-PLY and anti-CbpA titers were observed for mice administered MTRV001 containing test articles (Table 16). Anti-PLY IgG antibody titers were high in the sera of mice immunized with MTRV001 dose levels between 0.03 and 3 μg (reciprocal antibody titers between 160,000 and 640,000). However, mice immunized with 0.01 g MTRV001 developed a 32-fold lower anti-PLY titer compared to mice immunized with 0.03, 0.1, and 0.3 μg of MTRV001, and a 128-fold lower anti-PLY titer compared with mice immunized with 3 μg of MTRV001. High anti-PLY titers corresponding with high anti-hemolytic titers indicated a direct correlation of the ELISA titer with functional antibody titer. Anti-CbpA titers were lower than anti-PLY titers, as generally observed in all studies, but increased with higher MTRV001 dose levels; at the lowest dose level of MTRV001 administered (0.01 g), the anti-CbpA titer was no different than mice administered PBS.

A correlation between MTRV001 dose level and protection was clearly evident in this study. Mice immunized with higher dose levels of MTRV001 followed by challenge with a lethal dose of WT PLY toxin had increased rates of survival and increased geometric mean time to death (Table 16). For example, all (100%) of the mice administered 3 μg MTRV001 survived, whereas 80% of the mice administered 0.3, 0.1, and 0.03 μg MTRV001 survived. The groups administered either 3, 0.3, 0.1, or 0.03 μg MTRV001 had statistically significant survival compared to the PBS control group. For the group administered the lowest dose level of MTRV001 (0.01 μg), there was no significant difference in survival from WT PLY toxin challenge compared to the PBS control group. None of the mice administered PBS survived the IV challenge.

TABLE 16
Anti-PLY and Anti-CbpA IgG Titers, Anti-Hemolytic Titers,
and Survival following WT PLY Toxin Challenge

| | | Anti-PLY Response | | | |
|---|---|---|---|---|---|
| | Anti-CbpA | | WT PLY Challenge | | |
| | Response | Day 28 | Day 28 Anti- | | Geometric |
| | Day 28 | Anti-PLY | Hemolytic | % Survival | Mean TTD |
| Test Article | Anti-CbpA Titer | Titer | Titer | (1440 mpc) | (minutes) |
| 3 μg MTRV001 | 32,000 | 640,000 | 100 | 100% | 1440 |
| 0.3 μg | 4,000 | 160,000 | 50 | 80% | 481 |
| MTRV001 | | | | | |
| 0.1 μg | 4,000 | 160,000 | 75 | 80% | 336 |
| MTRV001 | | | | | |
| 0.03 μg | 80 | 160,000 | 37.5 | 80% | 464 |
| MTRV001 | | | | | |
| 0.01 μg | 10 | 5,000 | 37.5 | 20% | 20 |
| MTRV001 | | | | | |
| PBS Control | 10 | 10 | 25 | 0% | 4 |

CbpA = choline binding protein A; IgG = immunoglobulin G; mpc = minutes post-challenge; PBS = phosphate buffered saline; PLY = pneumolysin; TTD = time to death.

Based on the results from this study, for the anti-PLY immune response, the lowest MTRV001 dose level that yielded statistically significant protection from IV challenge with WT PLY toxin was 0.03 μg MTRV001. This group developed a 16,000-fold higher serum anti-PLY titer compared to PBS control group on Day 28 (2 weeks after the third and final immunization). For the anti CbpA antibody response, the titer was still increasing from the 0.3 μg to 3 μg (the highest dose tested in this study). However, the anti-CbpA titer results from the previous study demonstrated that a 3 μg MTRV001 dose plateaued in its anti-CbpA antibody response. Based on the results from these studies, the effective MTRV001 dose level for evaluating the immunogenicity/immunopotency of the CbpA moieties of MTRV001 was determined to be between 0.3 to 3 μg MTRV001 whereas for the anti-PLY response it was determined to be 0.03 to 0.3 μg MTRV001.

### Determination of Stability Indicating Dose Level of MTRV001 for Immunopotency Assay

The purpose of this study was to determine if effective MTRV001 dose levels previously identified for evaluating the anti-PLY and anti-CbpA antibody/immune responses are capable of detecting changes in MTRV001 that could impact immunopotency. Therefore, a dose-ranging study was performed to compare the immunogenicity and efficacy of MTRV001 subjected to thermal stress conditions versus an unstressed control.

To prepare a stressed sample of MTRV001, an MTRV001 sample (pre-adjuvanted at time of manufacture) was stored at 37° C.±4° C. for 3 months. A control sample was placed at the long term storage condition (5° C.±3° C.) in parallel. In this study, groups of mice were immunized IM once weekly for 3 injections (Days 0, 7, and 14) with 0.005, 0.05, and 0.5 μg immunization, mice were bled, sera were collected, and the anti-PLY and anti-CbpA titers were determined by ELISA on pooled sera from each group. Additionally, efficacy was assessed in a WT PLY toxin murine challenge model.

Dose-dependent anti-PLY and anti-CbpA titers were observed in the sera from mice immunized with either stressed or unstressed MTRV001; however, exposure to thermal stress resulted in significant decreases in both antibody titer and efficacy (Table 17). For example, at the 0.05 μg dose level, anti-PLY titers were 16-fold lower in the sera of animals that received stressed MTRV001 relative to the matched dose of unstressed MTRV001. Similarly, at the 0.005 μg dose level, anti PLY titers were >8-fold lower in the sera of animals that received stressed MTRV001 relative to the matched dose of unstressed MTRV001. When mice were challenged 2 weeks after the third immunization via IV administration of WT PLY toxin, mice immunized with the 0.05 μg dose of stressed MTRV001 had a 60% survival rate compared to 100% survival observed with mice administered 0.005 μg of unstressed MTRV001. This difference in survival correlated with the observed differences in anti PLY titers, whereby anti-PLY reciprocal antibody titers of 10,000 and 40,000 were observed for animals immunized with the stressed and unstressed (at one-tenth the dose level) MTRV001, respectively.

TABLE 17
Anti-CbpA IgG Titers, Anti-PLY IgG Titers, and Survival following
WT PLY Challenge for Mice Immunized with Stressed or Unstressed MTRV001

| | | | Anti-PLY Response | | | |
|---|---|---|---|---|---|---|
| | | | | WT PLY Challenge | | |
| | | Anti-CbpA | | | | Geometric |
| Test | | Response | Day 28 | Day 28 Anti- | | Mean |
| Article | MTRV001 | Day 28 Anti- | Anti-PLY | Hemolytic | % Survival | TTD |
| Condition | Dose | CbpA Titer | Titer | Titer | (1440 mpc) | (minutes) |
| Stressed | 0.5 μg | 400 | 80,000 | 50 | 100 | 1440 |
| (37 ± 4° C.) | 0.05 μg | 100 | 10,000 | 25 | 60 | 143 |
| | 0.005 μg | 100 | <5000 | 25 | 0* | 18 |
| Unstressed | 0.05 μg | 3200 | 160,000 | 200 | NT | NA |
| (5 ± 3° C.) | 0.005 μg | 100 | 40,000 | 50 | 100 | 1440 |

*p value of <0.001 when compared with unstressed 0.005 μg MTRV001 dose; log-rank Mantel-Cox Test.
CbpA = choline binding protein A;
IgG = immunoglobulin G;
mpc = minutes post-challenge;
NA = not available;
NT = not tested;
mpc = minutes post-challenge;
PBS = phosphate buffered saline;
PLY = pneumolysin;
TTD = time to death.

[symbol]

three injections, was selected for the immunization protocol to evaluate the anti-PLY titer for quality control testing of MTRV001.

In evaluating the CbpA response, the unstressed MTRV001 at the highest dose tested (0.05 μg) elicited an anti-CbpA titer of 3200 while stressed MTRV001 at this dose level elicited a background level response. Even at a 10-fold higher dose level (0.5 μg) of stressed MTRV001, an anti-CbpA titer of only 400 was observed. A dose level of 0.5 μg stressed MTRV001 corresponding to a CbpA titer of 400 was considered too low for the range of the assay. A dose 2-fold higher, 1 μg dose, administered weekly for three injections was selected for the immunization protocol to evaluate the anti-CbpA titer for quality control testing of MTRV001. A 1 μg dose is below the maximal CbpA titers observed at 3 μg with unstressed MTRV001 in prior studies.

### DISCUSSION

Disclosed herein is MTRV001, a protein-based pneumococcal vaccine candidate and a method of making and using the same. Through screening and selection of highly conserved pneumococcal protein antigens, PLY and CbpA emerged as antigens that could confer protection against a broad array of virulent pneumococcal strains and serotypes. MTRV001 is designed as a serotype-independent pneumococcal vaccine that confers protection well beyond the currently commercialized polysaccharide conjugate vaccines.

MTRV001 is an aluminum hydroxide adjuvanted recombinant fusion protein consisting of a PLY genetic toxoid and conserved CbpA peptide fragments at the N- and C-termini of the toxoid. The inclusion of both CbpA epitopes and the PLY genetic toxoid in MTRV001 is designed to elicit antibodies that inhibit the ability of S. pneumoniae to colonize and invade host tissues (anti-CbpA antibodies; upper and lower airway stages of pathogenesis) as well as neutralize PLY toxin, the primary cause of tissue damage, inflammation, and disease symptoms (anti-PLY antibodies; lower airway stages of pathogenesis).

A series of pharmacodynamic studies were performed to evaluate the in vitro and in vivo pharmacology of MTRV001. To evaluate whether immunization with MTRV001 and its precursor antigen, PLY-DM, could elicit protection against S. pneumoniae infection, a murine intranasal S. pneumoniae challenge model was utilized that mimics bacteremic pneumonia and sepsis. Immunization with PLY-DM demonstrated broad protective efficacy in this model providing significant protection against 17 of 20 S. pneumoniae serotypes (85%) that encompassed both Prevnar13® serotypes and emerging serotypes. Although the protective efficacy of MTRV001 was only assessed against challenge with four serotypes (19F, 6B, 22F nearly equivalent levels of protection from lethal pneumococcal challenge.

In a separate study, MTRV001 was evaluated for the protective capacity of CbpA epitopes in a S. pneumoniae intratracheal (IT) challenge model of infection. MTRV001 immunized mice exhibited significantly less lung pathology compared to unimmunized mice or mice immunized with PLY-DM, conclusively demonstrating the added protective value of the CbpA epitope(s). In support of these MTRV001 data, immunization of mice with YLN also protected lungs following challenge with S. pneumoniae.

The above-mentioned studies profiled the immunogenicity and efficacy of MTRV001. An adjuvant study was executed to determine the immunological impact of adjuvanting MTRV001 with aluminum hydroxide. Data from these studies indicate that adjuvanting MTRV001 with aluminum hydroxide engenders a more rapid induction of an antibody response compared to unadjuvanted MTRV001. Moreover, at a relatively low MTRV001 dose level (0.2 μg), aluminum hydroxide adjuvant enhanced both the magnitude of the anti-PLY antibody response as well as extended the durability of the anti-CbpA antibody response. Overall, the data support the use of aluminum hydroxide as an adjuvant to augment the immunogenicity of MTRV001.

Additional pharmacodynamic studies were conducted to define the optimal immunization regimen and MTRV001 dose level for evaluating PLY and CbpA immunopotency. The immunopotency assay will enable monitoring of MTRV001 drug substance and drug product during routine quality control testing. An important aspect of these studies was to determine if stress induced changes in MTRV001 could be reflected in the immunopotency assay. Therefore, a dose-ranging study was performed to compare the immunogenicity and efficacy of MTRV001 subjected to thermal stress conditions compared to an unstressed MTRV001 control. Based on the cumulative results from these dose ranging studies, 0.05 μg and 1 μg MTRV001 dose levels administered weekly for three injections, were selected as the immunization protocol to evaluate the anti-PLY and anti-CbpA IgG antibody titers, respectively, for the immunopotency related quality control testing of MTRV001.

In conclusion, the present application discloses in vitro and in vivo data demonstrating the superior efficacy of MTRV001 as a pneumococcal vaccine candidate over current methods. The MTRV001 nonclinical data package, including pharmacology and toxicology studies of MTRV001, collectively provide support for the clinical evaluation of MTRV001 in the proposed Phase 1 clinical study, described in Example 4.

### Example 2. Description of Manufacturing Process and Process Controls

MTRV001 is produced using a recombinant Escherichia coli cell line. The MTRV001 drug substance manufacturing process is initiated by the thaw and revival of two Master Cell Bank (MCB) vials. These cells are expanded in shake flasks to reach a cell density that is sufficient to inoculate the production fermentor. After reaching a target cell density in the production fermentor, the fermentation is induced with isopropyl β-D-1-thioglactopyranoside (IPTG). The cells are harvested by centrifugation, resuspended, and lysed to release the soluble MTRV001, then clarified, depth filtered and membrane filtered. The purification process consists of three chromatography columns (hydrophobic interaction, cation exchange and hydroxyapatite chromatography), anion exchange membrane filtration, and ultrafiltration/diafiltration (UFDF), followed by final formulation to adjust the concentration and add polysorbate 20. The formulated bulk drug substance is 0.2 m filtered, filled into sterile containers and stored at ≤−60° C.

An overview of the MTRV001 drug substance manufacturing process, including the fermentation and purification processes, is provided in Table 18. One batch of MTRV001 drug substance is derived from the purification of approximately half of the filtered harvest material from a ˜270 L production fermentation run.

### Description of the Fermentation Manufacturing Process

### Step 1: MCB Vial Thaw and Cell Culture Expansion

Following thaw of two MCB vials, the inoculum is expanded in sterile fermentation media supplemented with 0.04 g/L sterile kanamycin solution in shake flasks. The shake flasks are incubated with agitation at 37.0±2.0° C. to an optical density at 600 nm (OD600) of ≥6.0 AU/cm. Host purity of the final pooled flask material is assessed.

### Step 2: Production Fermentation

The pooled shake flask culture is used to inoculate the production fermentor containing sterile fermentation media supplemented with 40 μg/mL sterile kanamycin solution and 0.3 g/L antifoam. The production fermentor has a 300 L nominal volume and is operated with a working volume of ˜270 L.

During fermentation, the pH, dissolved oxygen (DO), temperature, agitation rate, and sparge gas flow rate are controlled to meet specified targets and ranges. Fermentation DO is may be added as needed during the fermentation process to avoid excessive foaming of the culture. Phosphoric acid solution and ammonium hydroxide solution are used to maintain a target pH of 7.2±0.2 during fermentation. The fermentation is maintained at 37.0±1.0° C. from inoculation through the induction phase. Induction of expression is initiated by the addition of 0.5 mM IPTG after a target OD600 of 16±3 AU/cm is achieved. Following a 4 hour induction period, the fermentor is cooled to 8.0° C. (range: 5.0° C.-15.0° C.) with reduced agitation and sparge rate. Host purity of the cooled fermentation material is assessed.

### Step 3: Harvest and Clarification

Cell harvest operations include collection of cells by centrifugation, then resuspension, and lysis of cells to release the soluble MTRV001, followed by clarification by centrifugation, then depth filtration and membrane filtration.

The cooled fermentation material is applied in aliquots to a disc stack centrifuge at 5.0-15.0° C. to collect the cells. The collected cells are resuspended in a sodium phosphate/sodium chloride buffer with temperature control at 8.0±3.0° C. and agitation of the resuspension pool. The resuspension pool is applied to a homogenizer to lyse the cells via high pressure to release the soluble product. The lysate is passed through the homogenizer three times with a temperature control target of 8° C. and agitation in the collection vessel. The resulting lysate is clarified via disc stack centrifuge at 20±5° C. to collect the supernatant. The supernatant pool is applied to a series of depth filters flushed with purified water before use then equilibrated in sodium phosphate/sodium chloride buffer.; a buffer chase is used to recover the hold-up volume. The collection vessel is maintained at 17-23° C. with agitation. The depth filtrate is 0.2 m filtered and collected at 5±3° C. with agitation. Filters may be replaced as needed to process the depth filtrate. Filtered harvest material is either held at 5±3° C. for further processing or filled into single-use, sterile bags for storage at <−60° C. for future use.

### Description of the Purification Manufacturing Process

Purification operations are performed at ambient temperature. The purification process is designed for end-to-end processing from the harvest filtrate to the drug substance and hold times during manufacture are minimized. Limited hold durations required for manufacture are supported by the experience gained during process development studies. Water for injection (WFI) is used at Step 5 or earlier; prior steps may utilize purified water.

### Step 4: Hydrophobic Interaction Chromatography

Hydrophobic interaction chromatography (HIC) is performed as a capture step and is intended to capture the MTRV001 product from the filtered harvest and reduce process- and bind/elute mode. This step may be performed in multiple cycles based on the resin load capacity and the amount of material to be processed.

Prior to use, the column is sanitized with a NaOH solution followed by a high salt Tris/NaCl equilibration buffer. The filtered harvest material is loaded with in-line conditioning using a high salt Tris/NaCl buffer and 0.2 m filtration. Following the load, the column is washed with equilibration buffer then a reduced salt buffer. The product is eluted in a gradient of decreasing salt concentration and the absorbance at 280 nm is monitored to guide peak collection. The HIC eluate may be stored at 5±3° C. for ≤12 hours during processing if necessary.

### Step 5: Anion Exchange Chromatoraphy

The anion exchange chromatography (AEX) step is performed as a polishing step and is intended to reduce process- and product-related impurities. This step utilizes a primary amino strong anion exchange resin and is operated in flow-though mode. This step may be performed in multiple cycles based on the resin load capacity and the amount of material to be processed.

Prior to use, the column is sanitized with a NaOH solution followed by pre-equilibration with a high salt sodium phosphate/NaCl buffer and then a sodium phosphate equilibration buffer. The HIC eluate is loaded with in-line conditioning using equilibration buffer and 0.2 m filtration. The load is chased with equilibration buffer and the product is collected in the flow-through with the absorbance monitored at 280 nm to guide peak collection. The AEX eluate may be stored at 5±3° C. for ≤12 hours during processing if necessary.

### Step 6: Hydroxyapatite Chromatography

Hydroxyapatite (HA) chromatography is performed as a polishing step and is intended to reduce process- and product-related impurities. The mixed mode resin effects molecular separations through a number of mechanisms including electrostatic, adsorption, weak ion exchange and calcium-based affinity interactions and is operated in bind/elute mode. This step may be performed in multiple cycles based on the resin load capacity and the amount of material to be processed.

Prior to use, the column is sanitized with a NaOH solution followed by pre-equilibration with a sodium phosphate buffer and then a sodium phosphate/sodium chloride equilibration buffer. The AEX pool is loaded with in-line conditioning using sodium phosphate buffer and 0.2 m filtration. Following the load, the column is washed with equilibration buffer. The product is eluted in a linear gradient of increasing salt concentration and the absorbance at 280 during processing if necessary.

### Step 7: Anion Exchange Membrane Filtration

Anion exchange membrane filtration is performed as a polishing step and is intended to reduce process-related impurities. The filter membrane is a salt tolerant interaction chromatography membrane with a primary amine ligand that is based on the principles of AEX and is operated in flow-through mode.

Prior to use, the filter is sanitized with a NaOH solution followed by pre-equilibration with a high salt sodium phosphate/sodium chloride buffer and then a sodium phosphate equilibration buffer. The HA pool is diluted in equilibration buffer prior to application and the load is chased with equilibration buffer. Product collection is based on fixed volumes of the pool filtration and chase steps.

### Step 8: UF/DF and Final Formulation

Ultrafiltration/diafiltration (UF/DF) is used to concentrate and buffer exchange the AEX membrane filtrate. A UF/DF membrane with a 30 kDa cutoff is flushed with WFI, sanitized with a NaOH solution, flushed with WFI, and finally equilibrated with diafiltration buffer (10 mM sodium phosphate, 154 mM NaCl, pH 7.4), prior to use.

The AEX membrane filtrate is initially concentrated to a target of 2.0 mg/mL, and then diafiltered with 10.0±1.0 diavolumes of diafiltration buffer. The retentate flow rate and transmembrane pressure are monitored throughout the UF/DF steps. Throughout the UF/DF and dilution steps, protein concentration is evaluated by absorbance at 280 nm. The diafiltered pool is combined with a post-recovery flush of the system, performed up to two times. A final dilution with diafiltration buffer may be performed to, adjust product concentration towards the final drug substance concentration of 1 mg/mL, allowing for a final addition of polysorbate 20 solution.

Polysorbate 20 stock solution (prepared in diafiltration buffer) is added to the UF/DF pool to a final concentration of 275 μg/mL and then mixed.

### Step 9: Filtration & Bulk Fill

The formulated bulk is 0.2 m filtered (filter is pre-equilibrated with 10 mM sodium phosphate, 154 mM NaCl, 275 μg/mL polysorbate 20, pH 7.4), with an initial volume discarded, and then filled into pre-sterilized PETG bottles in a laminar flow hood. The filter integrity is confirmed post-use. The MTRV001 drug substance is stored at ≤−60° C.

Reprocessing filter integrity test result or equipment failure. However, filtration will not be performed to remove confirmed microbial contamination or to mitigate other product quality issues. The final refiltered MTRV001 drug substance will be required to meet the release specification as previously described.

Exemplary results of the purification process are shown in Table 21, (“Process (with 3 column purification)). Altogether, this specific order of steps in the manufacturing process resulted in compositions comprising lower levels of impurities, such as media components, cells, cell debris, nucleic acids, host cell proteins (HCP), viruses, endotoxins, etc. that were suitable for therapeutic administration, which was not achievable by a two column purification. Furthermore, this process yielded a high proportion of full length monomeric forms of the immunogenic fusion protein in comparison to non-monomeric forms of the target molecule (e.g., immunogenic fusion protein) or non-full-length forms of the target molecule (e.g. N-terminal truncations of the immunogenic fusion protein). This provides a significant advantage for achieving a strong immunogenic response upon administration to a human subject for therapeutic purposes.

### Description of the In-Process Controls

In-process control testing for MTRV001 drug substance is performed at indicated steps

TABLE 19
Preliminary In-Process Control Testing for MTRV001 Drug Substance

| | | | Target or Acceptance | |
|---|---|---|---|---|
| Step | Test/Attribute | Method | Criteria | |
| 1 | Cell density | Optical density at 600 nm | ≤6.0 | AU/cm |
| 1 | pH | Potentiometric | Record value | |
| 1 | Inoculation flask pool purity | Host purity | No non-host micro- | |
| | | | organisms present | |
| 2 | Bioburden (pre-use | USP <61> | ≤1 CFU/10 mL | |
| | fermentation media) | | | |
| 2 | Cell density (to initiate gene | Optical density at 600 nm | 16 ± 3 | AU/cm |
| | expression induction with | | | |
| | IPTG) | | | |

| | | | |
|---|---|---|---|
| 2 | Final unprocessed fermentation | Host purity | No non-host micro- |
| | purity (cooled fermentation | | organisms present |
| | material) | | |
| 3 | Bioburden (filtered pool) | USP <61> | ≤100 CFU/10 mL |
| 3 | A₂₈₀ | Absorbance at 280 nm | Record value |
| 4 | Bioburden | USP <61> | ≤100 CFU/10 mL |
| 4 | Endotoxin | USP <85> | Record value |
| 4 | A₂₈₀ | Absorbance at 280 nm | Record value |
| 5 | Bioburden | USP <61> | ≤10 CFU/10 mL |
| 5 | Endotoxin | USP <85> | Record value |
| 5 | A₂₈₀ | Absorbance at 280 nm | Record value |
| 6 | Bioburden | USP <61> | ≤10 CFU/10 mL |
| 6 | Endotoxin | USP <85> | Record value |
| 6 | A₂₈₀ | Absorbance at 280 nm | Record value |
| 7 | Bioburden | USP <61> | ≤10 CFU/10 mL |
| 7 | Endotoxin | USP <85> | Record value |
| 7 | A₂₈₀ | Absorbance at 280 nm | Record value |
| 8 | Bioburden | USP <61> | ≤10 CFU/10 mL |
| 8 | Endotoxin | USP <85> | Record value |
| 8 | A₂₈₀ (initial, post-recovery | Absorbance at 280 nm | Record values |
| | flushes, final) | | |
| 9 | Filter integrity | Bubble point | Pass, per manufacturer's |
| | | | guidance for filter unit |

### Example 3. Manufacturing Process Development

### Overview of Manufacturing Process Development

The MTRV001 drug substance manufacturing process was initially developed with a 2-column purification process for non-GMP manufacture. In order to improve the clearance of process-related impurities, additional process development was performed, leading to the current process (3 column purification) described herein in EXAMPLE 2.

### Initial Process Development (Process Version 1a)

Initial process development was performed to establish conditions for fermentation, harvest and downstream purification. The initial process is termed version 1a for contextual clarity.

The manufacturing process was initiated by thawing a single vial of a 2nd generation EC-100 research cell bank (derived from the EC-100 RCB utilized to prepare master cell bank), expansion in shake flasks with media supplemented with kanamycin to a cell density sufficient to inoculate a 10 L production scale reactor. After reaching a target cell density in the production fermentor, the fermentation was induced with isopropyl β D 1 thioglactopyranoside (IPTG). The cells were harvested by centrifugation, frozen, resuspended in sodium soluble product. Following centrifugation, the supernatant was diluted and sterile filtered then applied sequentially to hydrophobic interaction and anion exchange chromatography resins. The purified material was buffer exchanged into a final formulation buffer (10 mM sodium phosphate, 154 mM NaCl, pH 7.4) and the drug substance was stored at 5±3° C. Note, for batch MTRV001 ENG01, 275 μg/mL polysorbate 20 was included in the formulation buffer. The polysorbate 20 concentration was selected based on general knowledge of effective polysorbate levels for stabilization of recombinant proteins and levels typically utilized in intramuscular injected recombinant products and vaccines.

Non-GMP batch GLPB-002, utilized to prepare the test articles used in the GLP toxicology study, was manufactured via the 2-column process described above and the analytical comparability relative to the proposed clinical drug substance batch CB-01. While process improvements were made to the process used for the GLP toxicology batch relative to the initial clinical process, the starting cell banks and the final product quality of the materials are similar.

### Process Transfer/Establishment of Initial Process (Process Version 1b)

The initial process incorporated changes to the raw materials/consumables, including transition to the MCB, and scale with associated necessary adjustments to the process (e.g., larger seed train volumes) were made, as well as other modifications required for facility fit/GMP production. As done with the later batch produced via previously mentioned process 1a, the drug substance was formulated at 10 mM sodium phosphate, 154 mM NaCl, 275 μg/mL polysorbate 20, pH 7.4.

Following product quality assessment of the drug substance produced from the process, it was concluded that additional process development was required to achieve drug substance with an appropriate process-related impurity profile suitable for clinical trial material. The key process related impurity to address was the elevated and unsuitable level of host cell protein (HCP). Material manufactured in this process 1b was, therefore, designated as non-GMP and solely used for reference standard and development studies.

### Process Development/Establishment of Initial Clinical Process (Process Version 2)

Process development encompassed both fermentation and purification manufacturing processes with the intent to improve the product quality of the drug substance, and in particular, host cell protein levels. Additionally, changes to raw materials/consumables and other modifications to the process as required for facility fit, were made. [symbol] accommodate the larger, 300 L production fermentation scale and the development of new harvest and clarification processes. Ammonium sulfate precipitation, acid precipitation, and depth filtration were evaluated for the harvest and clarification procedures, though ammonium sulfate and acid precipitation were found to have unsuitable product quality and yield, respectively. The final procedure incorporated the use of a disc stack centrifuge at the harvest stage to collect the cells from the bulk fermentation product and then again after homogenization to capture the soluble product. Depth filtration of the soluble product was also introduced to further remove particulates and improve the throughput of the membrane filtration prior to chromatographic processing. The selected fermentation, harvest and clarification process parameters from process development were employed in a 30 L pilot run for confirmation of the process for GMP production.

The purification process for MTRV001 drug substance was re-developed and process development included resin screening, development and optimization of chromatography for columns 1, 2, and 3, development of an anion exchange membrane filtration step, and ultrafiltration/diafiltration (UF/DF) development. Throughout process development, the aim was to optimize for removal of product- and process-related impurities, particularly HCPs. Resin screening included cation and anion exchange, heparin affinity, hydrophobic interaction, and hydroxyapatite resins. Resins were also evaluated with ammonium precipitation though it was subsequently observed to lead to increased product-related impurities. Following resin screening, chromatography parameters were developed and optimized, including parameters such as column load, elution program/buffer systems, excipient additions, pooling guidelines. The order of columns was also evaluated and the final scheme selected included hydrophobic interaction chromatography as the capture step followed by anion exchange and hydroxyapatite polishing chromatography steps. While the changes to the chromatography steps improved HCPs to target levels, an additional anion exchange membrane filtration step was developed and implemented to further improve HCP levels and provide additional capacity/process redundancy for HCP, endotoxin and host cell DNA clearance. The membrane selected is a salt tolerant interaction chromatography membrane with a primary amine ligand that is based on the principles of anion exchange chromatography and is operated in flow-through mode. UF/DF development was conducted with the aim of buffer exchanging/concentrating while maintaining target product quality, and included filter screening as well as load evaluations. The selected purification operations were employed in two 30 L pilot runs for confirmation of the process for GMP production. The storage condition for MTRV001 drug substance was support long-term product quality. The selected storage condition is supported by the available drug substance stability data and a development study evaluating freeze/thaw stability.

Manufacture of the non-GMP pilot run batch GLPB-0023 was performed using the previously described manufacturing process but with necessary scale and non-GMP facility modifications. Raw materials and consumables, including MCB, cell culture media, purification resins, depth filters and UF/DF filters were the same between lot GLPB-0023 and GMP batch CB-01, as possible. Batch analysis results for non-GMP batch GLPB-0023 and GMP batch CB-01 were evaluated.

### Development Batch Analysis and Analytical Comparability

Analytical results for the drug substance batches used in the GLP toxicology study (batch GLPB-002) and used to manufacture the clinical drug product (batch CB-01) are presented in Table 20. Of note, the target concentration of the drug substance has been modified during development and is diluted during drug product manufacture to the target drug concentration. While the result for percent main peak by size exclusion chromatography is 16% less for the clinical drug substance batch relative to the GLP toxicology batch, this is not considered critical to the function or safety of MTRV001 as the final drug product is adjuvanted resulting in a high density of MTRV001 on the adjuvant, likely as higher order species. Within the specified range of high molecular weight species (HMWS), there is no impact to the immunopotency of the final adjuvanted product and therefore the observed difference in percent main peak is not considered meaningful. The results demonstrate that batch GLPB-002, used for GLP toxicology, is comparable to the clinical drug substance batch CB-01, and this supports the use of batch CB-01 for manufacture of clinical drug product.

TABLE 20
Product Quality Comparison of the Batch Used for GLP
Toxicology and the Intended Clinical Drug Substance

| | | |
|---|---|---|
| | [symbol] | |
| | (Used to Prepare GLP | [symbol] |
| [symbol] | Toxicology Test Articles) | (Clinical Drug Substance Batch) |

General Tests

| | | |
|---|---|---|
| Appearance | Clear, colorless liquid | Clear, colorless liquid |
| pH | 7.4 | 7.1 |

Quantity

| | | | | |
|---|---|---|---|---|
| Protein concentration | 2.43 | mg/mL | 1.1 | mg/mL |

Identity

| | | |
|---|---|---|
| PLY | Positive, single band (Identity | Identity confirmed |
| | confirmed) | |
| CbpA-Y | Positive, single band (Identity | Identity confirmed |
| | confirmed) | |
| CbpA-N | Positive, single band (Identity | Identity confirmed |
| | confirmed) | |

Purity/Impurities

| | | |
|---|---|---|
| Purity/impurities by | 93% monomer | 100% monomer |
| SDS-PAGE | Single protein band migrating | Single protein band migrating |
| | similar to standard | similar to standard |
| Purity/impurities by | 93% main peak | 77% main peak |
| SEC-HPLC | 7% HMWS | 23% HMWS |

Potency

| | | |
|---|---|---|
| PLY immunopotency | 393,966 GMT (control: 10 GMT) ^{a} | 117,377 GMT (control: 10 GMT) |
| CbpA immunopotency | 15,647 GMT (control: 14 GMT) | 20,749 GMT (control: 10 GMT) |
| | ^{393,966} GMT (control: 10 GMT) ^{a} | |

Safety

| Endotoxin | 4.8 | EU/mg | <0.09 | EU/mg |
|---|---|---|---|---|
| Cytolytic activity | Toxicity undetected | | Toxicity undetected | |
| HCP | 377 | ng/mL | 27 | ng/mL |
| DNA | | | <41 | pg/mg |

[symbol]

indicates data missing or illegible when filed

Batch analysis results for additional development batches is provided in Table 21

TABLE 21
Batch Analysis for Development Batches

| | | | [symbol] |
|---|---|---|---|
| [symbol] | [symbol] | [symbol] | purification) |
| Test/Attribute | Process 1a | Process 1b | Process 2 |

General Tests

| | | | |
|---|---|---|---|
| Appearance | Clear, colorless liquid, | Clear, colorless liquid, | NT |
| | essentially free of | essentially free of | |
| | visible particles | visible particles | |
| pH | 7.4 | 7.3 | NT |

Quantity

| | | | | | | |
|---|---|---|---|---|---|---|
| Protein | 1.1 | mg/mL | 1.1 | mg/mL | 1.1 | mg/mL |

concentration

Identity

| | | | |
|---|---|---|---|
| PLY | Identity confirmed | Identity confirmed | Identity confirmed |
| CbpA-Y | Identity confirmed | Identity confirmed | Identity confirmed |
| CbpA-N | Identity confirmed | Identity confirmed | Identity confirmed |

Purity/Impurities

| | | | |
|---|---|---|---|
| Purity/impurities | 99% monomer; single | 98.1% monomer | 100% monomer; single |
| by SDS-PAGE | protein band migrating | | protein band migrating |
| | similar to standard | | similar to standard |
| Purity/impurities | 87.6% main peak | 84.1% main peak | 92% main peak |
| by SEC-HPLC | 9.8% HMWS | NR | 8% HMWS |

Potency

| | | | |
|---|---|---|---|
| PLY | 80,000 ^{a} | 28,537 | 33,936 |
| immunopotency | (control: 10 GMT) | (control: 10 GMT) | (control: 10 GMT) |
| CbpA | 64,000 ^{a} | 21,336 | 65,421 |
| immunopotency | (control: 40 GMT) | (control: 10 GMT) | (control: 10 GMT) |

Safety

| | | | | | | |
|---|---|---|---|---|---|---|
| Endotoxin | 17 | EU/mg | 1.9 | EU/mg | <0.1 | EU/mL |

| Cytolytic activity | Toxicity undetected | Toxicity undetected | | NT | |
|---|---|---|---|---|---|
| HCP | NA | 76,600 | ng/mg ^{b} | 30 | ng/mg |
| DNA | NT | 7 | pg/mg | <41.7 | pg/mg |

[symbol]

indicates data missing or illegible when filed

### Example 4: Stability Summary and Conclusions

### Summary of Stability Studies

The intended long-term storage condition for MTRV001 drug substance is ≤−60° C.

### Design of Stability Studies

Stability testing of MTRV001 drug substance is performed in accordance with ICH Q1A and ICH Q6B3. [symbol] and Table 23, respectively. Analytical procedures are the same as previously mentioned. Acceptance criteria for the GMP batch at the long-term condition (−75±10° C.) are defined by the specification in place at the time of study initiation. For these studies, MTRV001 drug substance was aliquoted into 5 mL PETG bottles with HDPE closures, which are representative of the bulk drug substance containers.

TABLE 12
Stability Protocol for Clinical Batch CB-01 (GMP)

| Storage | Time Point (Months) | | | | | | | | | |
|---|---|---|---|---|---|---|---|---|---|---|
| Condition | Initial | 0.5 | 1 | 3 | 6 | 9 | 12 | 18 | 24 | 36 |
| −75 ± 10° C. | A, B, | NT | A, B | A, B | A, B | A, B | A, B | A, B | A, B | A, B |
| 5 ± 3° C. | C ^{a} | NT | A, B | A, B | A, B, | A, B | A, B, | NT | NT | NT |
| | | | | | C | | C | | | |
| 25 ± 2° C./ | | A | A, B | NT | NT | NT | NT | NT | NT | NT |
| 60 ± 5% RH | | | | | | | | | | |

A: appearance, pH, protein concentration, purity/impurities by SEC-HPLC, purity/impurities by SDS-PAGE.
B: PLY and CbpA immunopotency.
C: polysorbate 20.
^{a} Batch release data is used for the initial time point.
NT = not tested.

TABLE 23
Stability Protocol for Batch NB11601p37 (Non-GMP)

| Storage | Time Point (Months) | | | | | | | | | |
|---|---|---|---|---|---|---|---|---|---|---|
| Condition | Initial | 0.5 | 1 | 3 | 6 | 9 | 12 | 18 | 24 | 36 |
| −75 ± 10° C. | A, B, | NT | A | A, B | A, B | A, B | A, B | A, B | A, B | A, B |
| 5 ± 3° C. | C ^{a} | NT | A | A, B | A, B, | A, B | A, B, | NT | NT | NT |
| | | | | | C | | C | | | |
| 25 ± 2° C./ | | A | A, B | NT | NT | NT | NT | NT | NT | NT |
| 60 ± 5% RH | | | | | | | | | | |

A: appearance, pH, protein concentration, purity/impurities by SEC-HPLC, purity/impurities by SDS-PAGE.
B: PLY and CbpA immunopotency.
C: polysorbate 20.
^{a} Batch release data is used for the initial time point.
NT = not tested.

### Discussions of Stability Data and Conclusion

The stability study for GMP batch CB-01 is ongoing at the long-term (−75±10° C.) storage condition and the 5±3° C. accelerated storage condition. Evaluations at the 25±2° C./60±5% RH accelerated storage condition have completed. For the long-term storage condition, available data for all tests have met the acceptance criteria and no significant trends or changes in attributes have been observed for any tests over the time points examined to date. At the 5±3° C. accelerated storage condition, the percent HMWS is trending upwards during storage. No examined to date. A similar but more pronounced trend is observed at the 25±2° C./60±5% RH accelerated storage condition with HMWS increasing 14% from initial storage to 1 month. While increasing HMWS has been observed at both accelerated storage conditions, the trend has not been observed at the long-term storage condition of −75±10° C.

The stability study for non-GMP batch NB11601p37 is ongoing at the long-term (−75±10° C.) storage condition and the 5±3° C. accelerated storage condition. Evaluations at the 25±2° C./60±5% RH accelerated storage condition have completed. For the long-term storage condition, no significant trends or changes in attributes have been observed for any tests over the time points examined to date. At the 5±3° C. accelerated storage condition, the percent HMW S is trending upwards during storage. No other significant trends or changes in attributes have been observed over the time points examined to date. A similar but more pronounced trend is observed at the 25±2° C./60±5% RH accelerated storage condition with HMWS increasing ˜15% from initial storage to 1 month. While increasing HMWS has been observed at both accelerated storage conditions, the trend has not been observed at the long-term storage condition of −75±10° C.

The available stability data support the long-term storage of MTRV001 drug substance at ≤−60° C. The stability of the drug substance will continue to be monitored in accordance with the stability protocols described herein.

### Example 4. A Phase 1, First-in Human, Randomized, Double-Blind, Placebo-Controlled, Dose-Escalation Study of the Tolerability, Safety, and Immunogenicity of MTRV001, a Pneumococcal Vaccine Candidate, in Healthy Adult Participants

### 1.1 Synopsis

Study Duration: The maximum planned duration is approximately 8.5 months for each participant, including screening (up to 28 days), treatment (2 doses administered approximately 1 month apart), and follow-up (assessments continuing for up to approximately 6.5 months after the second administration of study intervention).

Population: Healthy male or female adults ≥18 to ≤50 years of age for Cohorts 1 to 4 and ≥60 to ≤75 years of age for Cohort 5.

Investigational Product: MTRV001 drug product is a recombinant pneumococcal antigen, MTRV001, adjuvanted with aluminum hydroxide. MTRV001 drug product is formulated at 180 μg MTRV001/mL, 1 mg/mL aluminum (in the form of aluminum hydroxide) in 9 mM sodium phosphate, 139 mM sodium chloride, 275 μg/mL polysorbate 20, pH 7.4 [symbol] . form of aluminum hydroxide) in 9 mM sodium phosphate, 139 mM sodium chloride, pH 7.4.

Regimen and Dose by Cohort: Participants will receive 2 doses of MTRV001 or placebo in 1 of 5 cohorts.

TABLE 24
Planned Dosing Cohorts

| Cohort | N | | Dose of MTRV001^{a} |
|---|---|---|---|
| Cohort 1^{b} | 3 | (3 open-label) | 10 μg |
| Cohort 2^{b} | 12 | (2 open-label and 10 double-blind) | 30 μg |
| | 4 | | placebo |
| Cohort 3^{b} | 12 | (2 open-label and 10 double-blind) | 60 μg |
| | 4 | | placebo |
| Cohort 4^{b} | 12 | (2 open-label and 10 double-blind) | 90 μg |
| | 4 | | placebo |
| Cohort 5^{c} | 20 | (2 open-label and 18 double-blind) | TBD^{d} |

| | | |
|---|---|---|
| | 4 | placebo |
| Total | 75 | |

IRB: Institutional Review Board; SMC: Safety Monitoring Committee; TBD: to be determined.
^{a}If a particular dose level of MTRV001 is judged to be poorly tolerated, an intermediate dose of MTRV001 (i.e., a dose in between the previously tolerated dose and the poorly tolerated dose) may be evaluated, pending further review and concurrence by the SMC and the IRB.
^{b}Healthy adults ≥ 18 to ≤ 50 years of age.
^{c}Healthy adults ≥ 60 to ≤ 75 years of age, with ≥ 5 participants ≥ 67 to ≤ 75 years of age.
^{d}The dose of MTRV001 will be determined after reviewing tolerability and safety data from Cohorts 1 through 4.

### Study Objectives and Endpoints:

| | |
|---|---|
| Objectives | Endpoints |

Primary

| | |
|---|---|
| To assess the tolerability and the safety | Immediate reactogenicity events (local and systemic |
| profile of MTRV001 relative to each | adverse event [AEs]) within 30 minutes after each |
| ascending dose level of MTRV001 and | administration of study intervention. |
| relative to placebo. | Solicited reactogenicity events collected for 7 days after |
| | each administration of study intervention. |
| | AEs reported spontaneously by the participant up to |
| | 28 days after the last administration of study intervention. |
| | Serious adverse events (SAEs) and new-onset chronic |
| | illnesses (NOCIs) through Visit 7 (Day 210 [±14 days]). |
| | Changes from baseline in safety laboratory results at Visits |
| | 3 (Day 15 [±2 days]), 4 (Day 29 [±2 days]), and 5 (Day |
| | 43 [±4 days]). |
| | Assessment of vital signs. |
| of MTRV001 relative to each | immunoglobulin G antibody geometric mean titer as |
| ascending dose level of antigen and | measured by enzyme-linked immunosorbent assay (ELISA) |
| relative to the placebo. | at Visits 4 (Day 29 [±2 days]), 6 (Days 57 [±4 days]), and |
| | 7 (Day 210 [±14 days]). |

AE: adverse event; CbpA: choline binding protein A; ELISA: enzyme-linked immunosorbent assay; GMT: geometric mean titer; IgG: immunoglobulin G; NOCI: new-onset chronic illness; PLY: pneumolysin; SAE: serious adverse event

Study Design: This is a Phase 1, first-in-human, randomized, double-blind, placebo-controlled, dose-escalation study to assess the tolerability, safety, and immunogenicity of ascending doses of a pneumococcal vaccine candidate MTRV001. Potential participants will be screened within 28 days prior to Visit 2 (Day 1).

Approximately 75 participants who meet all inclusion and no exclusion criteria and provide written informed consent will be enrolled. Participants will receive 2 doses of MTRV001 or placebo in 1 of 5 cohorts as follows:

### Cohort 1:

3 participants ≥18 to ≤50 years of age will be enrolled to receive MTRV001 in an open-label and staggered manner.

Each participant's 72-hour safety data following administration of the first dose of study intervention will be reviewed by the Investigator and Sponsor prior to administration of the study intervention in the next participant.

In addition, the Safety Monitoring Committee (SMC) will review the tolerability and safety data of each participant within the cohort through Day 8 (7 days after administration of the first dose of study intervention), as well as any available immunogenicity data, to determine whether study intervention administration in the next cohort could start.

The second dose of study intervention will also be administered in an open-label and staggered manner, with each participant's 72-hour safety data following administration of the study intervention reviewed by the Investigator and Sponsor prior to administration of the study intervention in the next participant. The tolerability and safety data of each participant within the cohort will be monitored by the Investigator for 7 days after administration of the second dose of study intervention to ensure that no safety concerns are observed.

### Cohorts 2 to 4:

In each cohort, 16 participants ≥18 to ≤50 years of age will be enrolled to receive study intervention (12 participants to receive MTRV001 and 4 participants to receive placebo).

The first 2 participants will be enrolled to receive MXV01 in an open-label and staggered manner (sentinel group). Each participant's 72-hour safety data following Sponsor prior to administration of the study intervention in the next participant.

The remainder of the cohort will be randomized to receive study intervention in a blinded manner (10 participants to receive MTRV001 and 4 participants to receive placebo). Within each cohort, the SMC will review the tolerability and safety data of each participant through Day 8 (7 days after administration of the first dose of study intervention), as well as any available immunogenicity data, to determine whether study intervention administration in the next cohort could start.

The second dose of study intervention will also be administered in an open-label and staggered manner in the first 2 participants, with each participant's 72-hour safety data following administration of the study intervention reviewed by the Investigator and Sponsor prior to administration of the study intervention in the next participant. The tolerability and safety data of each participant within the cohort, as well as any available immunogenicity data, will be monitored by the Investigator for 7 days after administration of the second dose of study intervention to ensure that no safety concerns are observed.

### Cohort 5:

24 participants ≥60 to ≤75 years of age, with ≥5 participants ≥67 to ≤75 years of age, will be enrolled to receive study intervention (20 participants to receive MTRV001 and 4 participants to receive placebo).

The first 2 participants will be enrolled to receive MXV01 in an open-label and staggered manner (sentinel group). Each participant's 72-hour safety data following administration of the first dose of study intervention will be reviewed by the Investigator and Sponsor prior to administration of the study intervention in the next participant.

The remainder of the cohort will be randomized to receive study intervention in a blinded manner (22 participants to receive MTRV001 and 4 participants to receive placebo). Within the cohort, the tolerability and safety data of each participant will be monitored by the Investigator through Day 8 (7 days after administration of the first dose of study intervention), as well as any available immunogenicity data, to ensure that no safety concerns are observed.

The second dose of study intervention will also be administered in an open-label and staggered manner in the first 2 participants, with each participant's 72-hour safety data following administration of the study intervention reviewed by the Investigator and Sponsor prior to administration of the study intervention in the next participant. The tolerability and safety data of each participant within the cohort, as well as any available immunogenicity data, intervention to ensure that no safety concerns are observed

Should there be evidence of an unexpected AE, severe AE, or SAE (in each case considered at least possibly related to study intervention), the full SMC will be convened and asked to review the tolerability and safety data of the participants. In this case the decision to continue with administration of study intervention in the remainder of the cohort will be made after consultation with the full SMC.

Criteria to Proceed to the Next Cohort: The SMC will review all the tolerability and safety data through Day 8 (7 days after administration of the first dose of study intervention) for each dose cohort, as well as any available immunogenicity data, prior to initiation of administration of study intervention in the next cohort.

If a particular dose level is judged to be poorly tolerated, an intermediate dose of MTRV001 (a dose in between the previously tolerated dose and the poorly tolerated dose) may be evaluated, pending further review and concurrence by the SMC and the IRB.

Review of safety and tolerability data, as well as any available immunogenicity data, for the first 4 cohorts will be performed prior to selecting the dose for Cohort 5. The dose of study intervention for Cohort 5 will be selected as the highest tolerated dose administered in Cohorts 1 through 4.

Study Halting Rules: The following study halting rules will apply:

1. If 1 participant in any cohort experiences a Grade 4 AE or SAE at least possibly related to the study intervention, administration of study intervention will be suspended for that cohort until a full safety review is performed.

2. If ≥2 participants in any cohort experience the same Grade 3 AE (not including vital signs AEs [i.e., heart rate, respiratory rate, and blood pressure only]) that cannot be clearly attributed to other causes, administration of study intervention will be suspended for that cohort until a full safety review is performed. If ≥3 participants of any cohort experience a Grade 3 vital signs AE that cannot be clearly attributed to other causes, administration of study intervention will be suspended for that cohort until a full safety review is performed.

Study intervention will be administered by intramuscular (I) injection in the deltoid muscle of the non-dominant arm (or dominant arm if participant prefers) at Visits 2 (Day 1) and 4 (Day 29 [±2 days]) at the study site. Participants will be observed by study personnel at the study site for 30 minutes following each administration of study intervention, and any AEs will be recorded [symbol] . provided thermometers) and local and systemic AEs daily for 7 days after each administration of study intervention. Participants will also be provided a ruler for assessment of the injection site. All AEs/SAEs and NOCIs will be recorded from the time of administration of the first dose of study intervention through Visit 6 (Day 57 [±4 days]). Thereafter, only SAEs and NOCIs will be recorded through Visit 7 (Day 210 [±14 days]). AEs reported after signing of the ICF and prior to administration of the first dose of study intervention will be considered medical history.

Site clinic visits will occur at Screening and Days 1, 15 (±2 days), 29 (±2 days), 43 (±4 days), 57 (±4 days), and 210 (±14 days). Protocol-specified safety laboratory tests will be performed at Screening and Days 1 (pre-dose), 15 (±2 days), 29 (±2 days [pre-dose]), and 43 (±4 days). Blood samples will be taken for immunogenicity assessments on Days 1 (pre-dose), 29 (±2 days; pre-dose), 57 (±4 days), and 210 (±14 days).

Each participant will be contacted by telephone at approximately 24 hours, 72 hours, 7 days, and 13 days following each administration of study intervention for safety assessments and review of the diary. Between Visits 6 (Day 57 [±4 days]) and 7 (Day 210 [±14 days]), each participant will be contacted by telephone at monthly intervals (Days 90, 120, 150, 180 [±3 days]) for safety follow-up. A healthcare professional, such as a registered nurse, nurse practitioner, or physician's assistant, at the study site will record the study telephone calls using a scripted interview questionnaire for the first 7 days following each administration of study intervention. Other telephone calls may be performed and recorded by trained study staff.

When data from all cohorts (Cohorts 1 to 5) through Visit 6 (Day 57 [±4 days]) are available and monitored, analysis of safety, tolerability, and immunogenicity data will be conducted. These analyses will constitute the primary content of the clinical study report (CSR). All study participants will remain blinded. The Investigator or designee and a blinded subgroup of study personnel at the site involved in the ongoing conduct of the study (following Visit 6 [Day 57 (±4 days)]) will remain blinded to study intervention. The blinded Investigator or designee will assess the relationship of any SAEs or NOCIs reported after Visit 6 (Day 57 [±4 days]).

All safety and immunogenicity data after Visit 6 (Day 57 [±4 days] will be subsequently summarized and added to the CSR as an addendum.

Study site personnel will follow the site's COVID-19 policy that is in place at the time of study conduct.

### Study Population:

### Inclusion:

Participants must meet all of the following criteria to be eligible:
1. Participant must be male or female ≥18 to ≤50 years of age for Cohorts 1 to 4 and ≥60 to ≤75 years of age for Cohort 5, at the time of signing the informed consent.
2. Body mass index within the range 18 to 32 kg/m² (inclusive).
3. Participants who are free of clinically significant acute or chronic health conditions in the opinion of the Investigator.
4. Have provided written informed consent prior to screening procedures.
5. Participant's screening laboratory test results must be either within the normal range or deemed as not clinically significant by the Investigator.
6. Venous access considered adequate for collection of safety laboratory samples and immunogenicity samples.
7. Contraceptive use by males and females should be consistent with local regulations regarding the methods of contraception for those participating in clinical studies. In addition, women of childbearing potential must agree to avoid heterosexual activity for a period of 14 days prior to the administration of study intervention.

The Investigator evaluates the effectiveness of the contraceptive method in relationship to the first dose of study intervention.

The Investigator reviews the medical history, menstrual history, and recent sexual activity to decrease the risk for inclusion of a female with an early undetected pregnancy.

### Exclusion:

Participants will be excluded from the study if any of the following criteria apply:
1. Positive screening test suggesting a current infection due to human immunodeficiency virus, hepatitis C virus, or hepatitis B virus infection.
2. Suspected or known alcohol and/or illicit drug abuse within the past 5 years.
3. Regular use of tobacco- or nicotine-containing products within 6 months prior to screening.
4. Electrocardiogram abnormalities outside of accepted ranges (with some exceptions) or results considered to be clinically significant. Participants with QT interval corrected for heart rate according to Fridericia's formula >450 msec (if male) or >460 msec (if female) will be excluded.
5. History of confirmed pneumococcal infection based on participant report of medical history during the previous 12 months. [symbol] investigational drug prior to receiving the first dose of study intervention. All investigational (non-registered) drugs used should be noted.
7. Use of chronic immunosuppressant agents or other immune-modifying drugs within 6 months prior to receiving the first dose of study intervention. Short-term use of corticosteroids (<14 days) for an acute illness are allowed but last dose should be ≥28 days prior to administration of the first dose of study intervention. The use of topical, inhaled, and nasal glucocorticoids is permitted.
8. Receipt of immunoglobulins and/or any blood products within the 3 months preceding Day 1 or planned administration of such products during the study and up Visit 6 (Day 57 [±4 days]).
9. Is planning to become pregnant in the time period from Screening up to 30 days following the last dose of study intervention.
10. History of allergic disease, neurologic disease, or untoward reactions likely to be exacerbated by any component of the vaccine and/or known hypersensitivity to any component of the vaccine.
11. Any condition that in the opinion of the Investigator would pose a health risk to the participant if enrolled or could interfere with evaluation of the study intervention or interpretation of study results (including neurologic or psychiatric conditions deemed likely to impair the quality of safety reporting).
12. Known or suspected immunological dysfunction.
13. History of administration of any vaccine within 30 days of receiving the first dose of study intervention. Should a vaccine have been administered within the 30-day timeframe, inclusion into the study will be at the discretion of the Investigator. Vaccines may not be administered until after Visit 6 (Day 57 [±4 days]).
14. Unwilling or unable to forego donation of sperm, egg, blood, plasma, or platelets from Screening through Visit 6 (Day 57 [±4 days]).
15. In the opinion of the Investigator, any participant with a physical or laboratory finding or past medical history that might suggest a good quality of life for the participant is likely to be <24 months at the time of Screening examination.
16. Participants who, in the opinion of the Investigator, will not be able to comply with all the study procedures and visits as outlined in the protocol, including follow-up.
17. A staff member or family member of a staff member of the clinical research organization.

The following study halting rules will apply:
1. If 1 participant in any cohort experiences a Grade 4 AE or SAE at least possibly related to the study intervention, administration of study intervention will be suspended for that cohort until a full safety review is performed.
2. If ≥2 participants in any cohort experience the same Grade 3 AE (not including vital signs AEs) that cannot be clearly attributed to other causes, administration of study intervention will be suspended for that cohort until a full safety review is performed. If ≥3 participants of any cohort experience a Grade 3 vital signs AE that cannot be clearly attributed to other causes, administration of study intervention will be suspended for that cohort until a full safety review is performed.

### Data Analysis:

At the conclusion of Visit 6 (Day 57 [±4 days]) of Cohort 5, the database will be “locked” for analysis when (a) all participants' safety data have been entered and all data queries have been resolved; and (b) ELISA based results from immunogenicity sera samples taken through Visit 6 (Day 57 [±4 days]) are available.

All participants and study personnel involved in further safety follow-up (through Visit 7 [Day 210 (±14 days)]) will remain blinded to treatment assignment, and all safety data collected following Visit 6 (Day 57 [±4 days]) will be reported in a safety addendum appended to the initial CSR when available.

After bioanalysis of Visit 7 (Day 210 [±14 days]) specimens are available, the remaining data will be added to the CSR by an addendum.

The following populations are defined:

Intent-to-treat population: All participants who are considered eligible for participation and are enrolled in the study.

Safety population: All participants who are enrolled in the study and receive ≥1 dose of study intervention. Safety analyses will be based on this population.

Per protocol population: All participants who receive both doses of study intervention at Visits 2 (Day 1) and 4 (Day 29 [±2 days]) and have Visits 2 (Day 1), 4 (Day 29 [±2 days]), and 6 (Day 57 [±4 days]) ELISA data. Participants will be analyzed per the actual study intervention they received. All immunogenicity analyses will be based on this population.

Sample Size: Approximately 75 participants are planned to be included in the study, 3 participants in Cohort 1, 16 participants each in Cohorts 2 through 4, and 24 participants in provide sufficient data for assessment of the study objectives.

### Statistical Methods:

All analyses and data presentations will be descriptive in nature. All study data will be included in the individual participant data listings. All summary tables will present descriptive statistics for the parameters to be analyzed by cohort, wherever applicable.

Data from participants who undergo open-label MTRV001 will be presently separated from randomized participants. Data for participants assigned to placebo treatment may be pooled. Generally, tabular summaries will present results by cohort and placebo.

Tolerability and safety will be assessed by tabulating the frequency, duration, and severity of reactogenicity events, as well as tabulating overall treatment-emergent adverse events (TEAEs), SAEs, AEs leading to discontinuation, NOCIs, changes in laboratory parameters, and assessments of vital signs. AEs will be tabulated and characterized Medical Dictionary for Regulatory Activities system organ class and preferred term, intensity, and causality to study intervention. Changes from baseline in laboratory assessments will be summarized descriptively. Vital sign assessments will be summarized descriptively. Descriptive statistics will be presented for each cohort and summarized across all cohorts.

Immunogenicity data will be summarized by treatment group according to the endpoints. Seroconversion rates, geometric mean titers, and geometric mean fold rises (post-/pre-) will be tabulated and graphically summarized.

### 1.2 Schemas

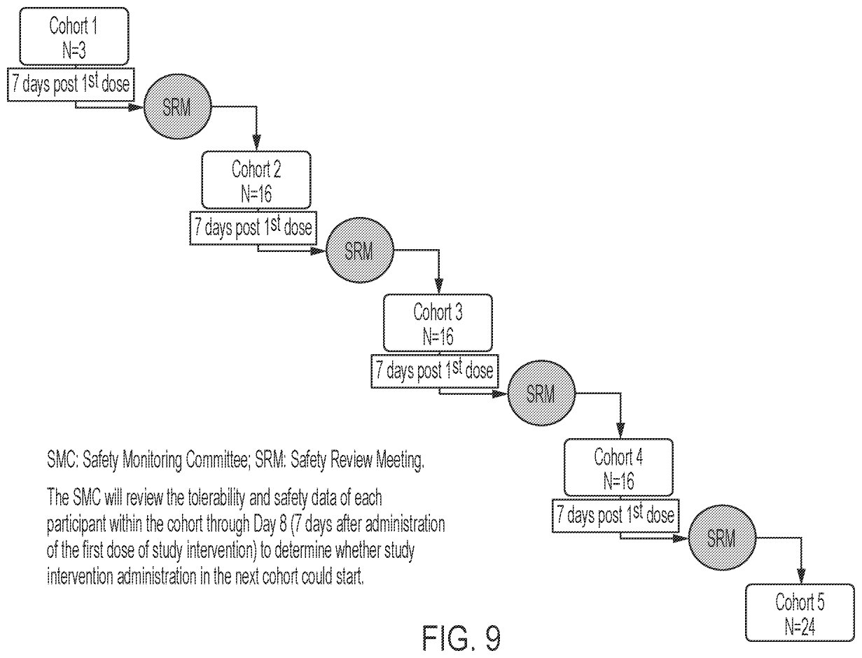

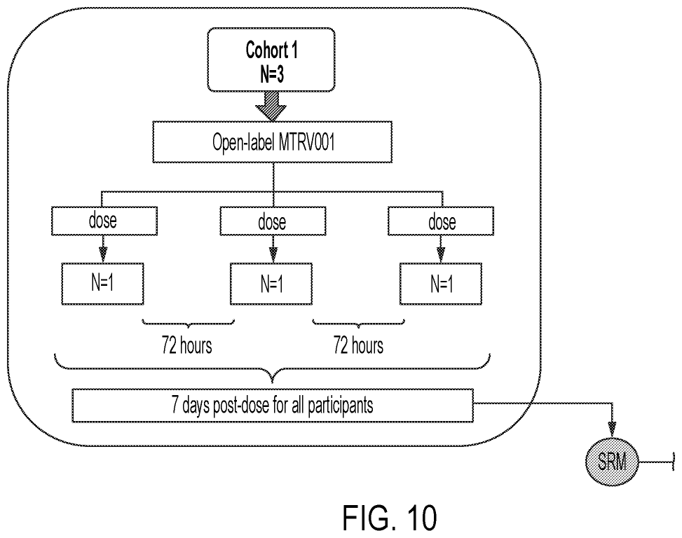

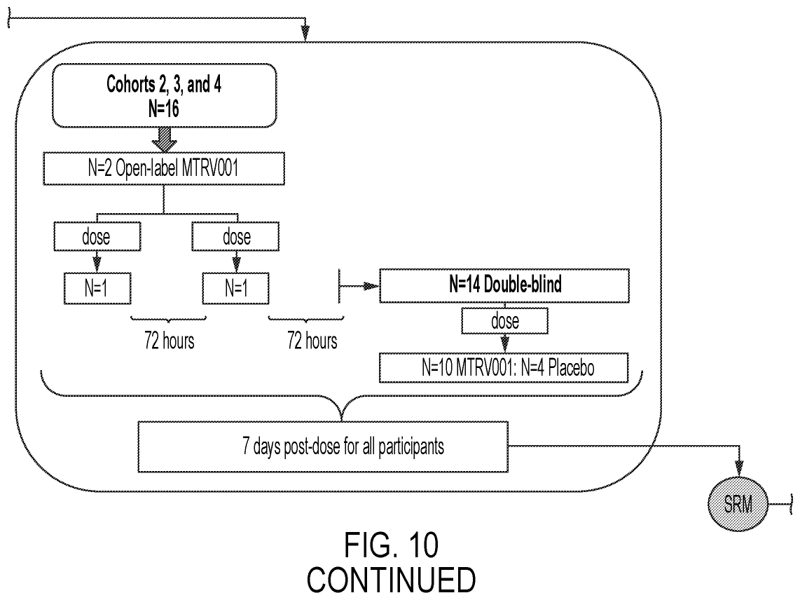

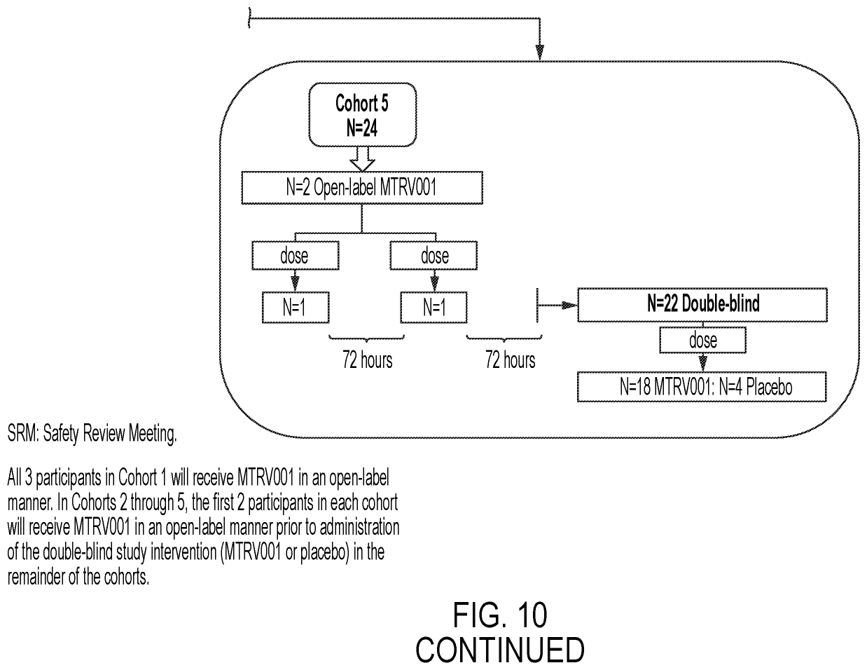

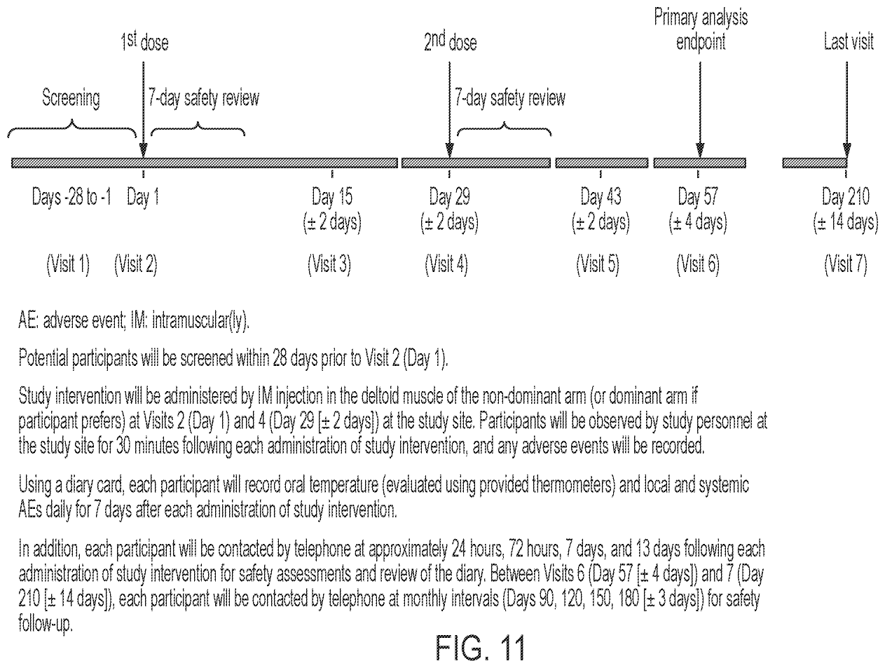

The overall study schema is presented in FIG. 9, the study intervention administration schema is presented in FIG. 10, and the participant timeline is presented in FIG. 11

### Schedule of Activities

TABLE 25
Schedule of Activities for Site Clinical Visits

| | Clinic Visit | | | | | | | |
|---|---|---|---|---|---|---|---|---|
| | 1 | 2 | 3 | 4 | 5 | 6 | 7 | |
| | Study Day | | | | | | | Early |
| | | | Day | Day | Day | Day | Day | Termination |
| | Screening | | 15 | 29 | 43 | 57 | 210 | Visit (if this |
| | Days −28 | Day | (±2 | (±2 | (±2 | (±4 | (±14 | visit is prior |
| | to −1 | 1 | days) | days) | days) | days) | days) | to Visit 5)^{a} |
| Informed consent | X | | | | | | | |
| Review inclusion/exclusion | X | X | | | | | | |
| criteria | | | | | | | | |
| Medical history | X | | | | | | | |
| Targeted review of medical | | X | | X | | | | |
| history | | | | | | | | |
| Prior/concomitant | X | X | X | X | X | X | X | X |
| medications | | | | | | | | |
| Height | X | | | | | | | |
| Weight/BMI | X | X | | | | | | |
| Vital signs^{b} | X | X | X | X | X | X^{c} | X^{c} | X |
| Complete physical | X | | | | | | | |
| examination^{d} | | | | | | | | |
| Targeted physical | | X | X | X | X | X | X^{c} | X |
| examination^{e} | | | | | | | | |
| ECG^{f} | X | | | | | | | |
| Safety laboratory tests | X | X | X | X | X | X^{c} | X^{c} | X |
| (hematology, chemistry, | | | | | | | | |
| and urinalysis)^{g} | | | | | | | | |
| Urine pregnancy test | X | X | | X | | X | X^{c} | X |
| HIV, HBV, and HCV tests | X | | | | | | | |
| Enrollment/Randomization^{h} | | X | | | | | | |
| Immunogenicity sampleⁱ | | X | | X | | X | X | X |
| Administration of study | | X | | X | | | | |
| intervention | | | | | | | | |
| Dispense diary cards | | X | | X | | | | |
| Review diary cards | | | X | | X | | | X^{j} |
| Reactogenicity assessment | | X | | X | | | | |
| AEs, SAEs, and NOCIs^{l} | | X | X | X | X | X | X | X |

AE: adverse event;
BMI: body mass index;
ECG: electrocardiogram;
HBV: hepatitis B virus;
HCV: hepatitis C virus;
HIV: human immunodeficiency virus;
NOCI: new-onset chronic illness;
SAE: serious adverse event.
^{a}If a participant withdraws from the study prior to Visit 5 (Day 43 [±2 days]), attempts should be made to undergo the Early Termination Visit assessments. If the participant discontinues after Visit 5 (Day 43 [±2 days]), attempts should be made to perform Visit 7 (Day 210 [±14 days]) assessments.
^{b}Vital signs include blood pressure, heart rate, respiratory rate, and oral temperature. At Visits 2 (Day 1) and 4 (Day 29 [±2 days]), vital signs to be obtained before and 30 minutes (±5 minutes) after administration of study intervention. Participants should be seated, and the assessments performed after 5 minutes of rest.
^{c}Optional assessments to be performed at Investigator discretion.
^{d}A complete physical examination includes clinical assessments of head, ears, eyes, nose, and throat; neck; lymph nodes; heart; chest; abdomen; extremities; neurological function; skin; and joint/arthritis evaluation.
^{e}A targeted physical examination includes appropriate examination based on participant self-reported symptoms or complaints.
^{f}12-lead ECGs will be performed in triplicate after the participant has been resting in the supine position for ≥5 minutes.
^{g}Samples to be drawn pre-dose on dosing days. Erythrocyte sedimentation rate will be assessed at screening in addition to the protocol-specified tests.
^{h}All 3 participants in Cohort 1 will receive MTRV001 in an open-label manner. In Cohorts 2 through 5, the first 2 participants in each cohort will receive MTRV001 in an open-label manner prior to administration of the double-blind study intervention (MTRV001 or placebo) in the remainder of the cohort.
ⁱImmunogenicity samples to be drawn pre-dose on dosing days. [symbol]
[±2 days]).
^{k}Reactogenicity at Visits 2 (Day 1) and 4 (Day 29 [±2 days]) will be assessed by site staff for 30 minutes following administration of study intervention (in addition to any AE). Participants will assess reactogenicity for 7 days following each administration of study intervention with diary cards which will be reviewed in conjunction with site staff at Visits 3 (Day 15 [±2 days]) and 5 (Day 43 [±2 days]). Reactogenicity events will include: injection site pain, tenderness, erythema/redness, pruritus/itching, induration/hardening, swelling, fatigue, fever, rash, headache, myalgia/muscle pain, nausea, vomiting, and flu-like symptoms.
^{l}Only SAEs/NOCIs will be monitored for and reported after Visit 6 (Day 57 [±4 days]) and through Visit 7 (Day 210 [±14 days]). Any AEs reported prior to administration of the first dose of study intervention will be recorded as medical history. [symbol]
indicates data missing or illegible when filed

TABLE 26
Schedule of Activities for Telephone Calls

| | Telephone Call Assessments^{a} | | | | |
|---|---|---|---|---|---|
| | 24 Hours | 72 Hours | 7 Days | 13 Days | |
| | Following | Following | Following | Following | Monthly |
| | Each Dose | Each Dose | Each Dose | Each Dose | Calls |
| | Study Day | | | | |
| | | | | | Days 90, 120, |
| | Day 2 | Day 4 | Day 8 | Day 14^{c} | 150, 180 (±3 |
| | Day 30^{b} | Day 32^{b} | Day 36^{b} | Day 42^{b,c} | days) |
| AEs/scripted questions | X | X | X | X | |
| Diary card reminders | X | X | X | X | |
| SAEs or | X | X | X | X | X |
| NOCIs/scripted | | | | | |
| questions | | | | | |
| Concomitant | X | X | X | X | X |
| medications | | | | | |

AE: adverse event; NOCI: new-onset chronic illness; SAE: serious adverse event.
^{a}A healthcare professional such as a registered nurse, nurse practitioner, or physician's assistant will complete the telephone calls over the first 7 days following each administration; other telephone calls may be performed by trained study staff. Presented as approximate times.
^{b}In-clinic Visits 3 and 5 (Days 15 and 43, respectively) have windows of ±2 and ±4 days, respectively; if the participant returns to the site earlier than the planned telephone calls for Days 14 or 42, the planned telephone call is unnecessary.
^{c}The second dose of study intervention will be administered at the in-clinic Visit 4 (Day 29), which has a window of ±2 days; if the second dose is administered 1 or 2 days before or after Day 29, the post-dose telephone call schedule should be adjusted accordingly so that calls occur at approximately 24 hours, 72 hours, 7 days, and 13 days following the dose.

TABLE 27
Risk Assessment

| Identified and | Summary of | |
|---|---|---|
| Potential Risks of | Data/Rationale for | |
| Clinical Significance | Risk | Mitigation Strategy |
| Solicited | Potential anticipated | A number of measures will be employed in this study for |
| reactogenicity AEs | risks associated with | MTRV001 to ensure safety and to monitor for potential |
| including local | MTRV001 | local and systemic AEs, including: |
| reactogenicity events | administration are | Dose-escalation study design, with increased dose level |
| such as pain, redness, | consistent with those | in each cohort with Cohort 5 receiving the highest |
| and swelling at the | associated with other | tolerated dose administered in Cohorts 1 through 4. |
| injection site and | vaccines. | Open-label, sentinel administration of the 3 participants |
| systemic | | in Cohort 1 and the first 2 participants in Cohorts 2 |
| reactogenicity effects | | through 5, with 72-hour safety monitoring, prior to |
| such as fatigue, fever, | | dosing in the next participant. |
| headache, and flu like | | Safety evaluations including assessment of local and |
| symptoms. | | systemic reactogenicity events, AEs reported through |
| | | Day 57, SAEs and NOCIs reporting through Day 210, |
| | | clinical laboratory tests, and vital signs. In addition to |
| | | immediate reactogenicity events observed within |
| | | 30 minutes after each dose, solicited reactogenicity |
| | | events will be collected via diary cards for 7 days after |
| | | each dose. A comprehensive set of clinical laboratory |
| | | tests including hematology, chemistry, urinalysis, and |
| | | other special tests will be performed. A targeted review |
| | | of changes in medical history will be performed prior to |
| | | dosing at Days 1 and 29; any changes following the first |
| | | dose will be considered AEs. Targeted physical |
| | | examinations will also be performed. |
| | | SMC oversight with review of tolerability and safety |
| | | data through Day 8 (7 days after administration of the |
| | | first dose of study intervention) to determine whether |
| | | study intervention administration in the next cohort |
| | | could start as well as if there is evidence of an |
| | | unexpected AE, severe AE, or SAE (in each case |
| | | considered at least possibly related to study |
| | | intervention). |
| | | Protocol-specified halting rules. |
| | | In addition, the model ICF clearly describes the |
| | | requirements of the study protocol, including the risks |
| | | associated with study procedures and the potential adverse |
| | | effects of the study intervention. The need for pregnant |
| | | women to be excluded from study participation is described |
| | | the ICF. The ICF meets ICH E6 (GCP) standards and |
| | | contains all required elements of informed consent as |
| | | described in 21 CFR 50.25. |
| Safety of MTRV001 | In the repeat-dose | Three doses of MTRV001 at each dose level were |
| administered in 2 | toxicology study in | administered to rabbits in the toxicology study to provide |
| doses to human | NZW rabbits, IM | excess exposure of the test subjects to the test material |
| participants in the | administration of | (ICH M3 (R2)). The 3 dose levels of the test article |
| Phase 1 clinical | MTRV001 at target | adjuvanted with 0.5 mg Alhydrogel ® bracket the 4 |
| study. | doses of 10, 30, and | proposed dose levels and used the same route of |
| | 90 μg (±0.5 mg | administration (IM) planned for the Phase 1 clinical study. |
| | adjuvant) did not | |
| | result in any | |
| | treatment-related, | |
| | toxicologically | |
| | significant, or | |
| | adverse findings in | |
| | rabbits following 3 | |
| | injections on Study | |
| | Days 1, 15, and 29. | |
| | Therefore, the | |
| | NOAEL was 90 μg | |
| | MTRV001 (±500 μg | |
| | adjuvant), the highest | |
| | dose level studied. | |
| SARS-CoV-2 | As for the general | During the entire study, all recommendations issued by |
| infection for study | population, there is a | WHO as well as local guidelines will be followed with |
| participants as long | risk of a SARS-CoV- | respect to the minimization of the risk of disease spreading, |
| as the COVID-19 | 2 infection for study | e.g., social distancing, disinfection, hygiene, and wearing of |
| pandemic situation is | participants as long | appropriate mouth-nose masks. During the pandemic |
| ongoing. | as the COVID-19 | situation, further measures according to recommendations |
| | pandemic situation is | and requirements from local Health Authorities may become |
| | ongoing. | necessary and will be followed within the context of this |
| | | study as far as applicable, in order to ensure full |
| | | implementation of the principles of GCP with priority on |
| | | participant safety in this study also during the COVID-19 |
| | | pandemic situation. These measures are described in a |
| | | preventive action plan implemented at the Investigator site. |
| | | In order to minimize the risk coming from a current |
| | | infection and the risk of getting infected by other |
| | | participants during the in-house periods of the study, the |
| | | following measures are to be implemented: Only |
| | | participants without any symptoms of a respiratory disease |
| | | and without contact to any known SARS-CoV-2 positive |
| | | patient or COVID-19 patient will be included into the study. |
| | | Furthermore, as part of the clinical study procedures, |
| | | participants will be closely monitored (including for signs of |
| | | COVID-19) during the entire study duration. The |
| | | continuation of the study in the case of a SARS-CoV-2 |
| | | infection in a study participant, in an identified contact to a |
| | | SARS-CoV-2 positive participant, or COVID-19 patient |
| | | will be decided at the Investigator's discretion and in |
| | | agreement with the medical monitoring team. The Sponsor |
| | | will monitor the events related to any SARS-CoV-2 |
| | | infection reported following study intervention |
| | | administration regularly and update the recommendations, if |
| | | necessary. |

COVID-19: coronavirus disease 19; GCP: Good Clinical Practice; ICH: International Council for Harmonisation; IM: intramuscular(ly); SARS-CoV-2: severe acute respiratory syndrome coronavirus 2; WHO: World Health Organization.

TABLE 28
Solicited Reactogenicity Adverse Events

| Local Expected Adverse Events (at Injection Site) | Systemic Expected Adverse Events |
|---|---|
| Erythema/redness | Fatigue |
| Induration/hardening | Fever |
| Pain | Flu-like symptoms |
| Pruritus/itching | Headache |
| Swelling | Myalgia/muscle pain |
| Tenderness | Nausea |
| | Rash |
| | Vomiting |

All 3 participants in Cohort 1 will receive MTRV001 in an open-label manner. In Cohorts 2 through 5, the first 2 participants in each cohort will receive MTRV001 in an open-label manner prior to administration of the double-blind study intervention (MTRV001 or placebo) in the remainder of the cohort.

MTRV001 (containing 10, 30, 60, or 90 μg of MTRV001 per dose) or placebo will be injected IM in the deltoid muscle of the non-dominant arm (or dominant arm if participant prefers) at Visits 2 (Day 1) and 4 (Day 29 [2 days]). Participants must be seated in an armchair during administration of study intervention.

MTRV001 drug product is an aluminum hydroxide adjuvanted recombinant pneumococcal protein antigen. MTRV001 drug product is formulated at 180 g MTRV001/mL, 1 mg/mL aluminum (in the form of aluminum hydroxide) in 9 mM sodium phosphate, 139 mM sodium chloride, 275 μg/mL polysorbate 20, pH 7.4. MTRV001 drug product is a white to off-white cloudy suspension. Each single-dose vial of MTRV001 drug product contains ≥1 mL. MTRV001 drug product is manufactured via a recombinant bacterial (Escherichia coli) expression system.

MTRV001 placebo is formulated at 1 mg/mL aluminum (in the form of aluminum hydroxide) in 9 mM sodium phosphate, 139 mM sodium chloride, pH 7.4. MTRV001 placebo is a white to off-white cloudy suspension. Each single-dose vial of MTRV001 placebo contains ≥2 mL.

Vials of MTRV001 drug product and MTRV001 placebo are stored at 5° C.±3° C. Do not freeze.

MTRV001 drug product and MTRV001 placebo should be thoroughly mixed by inversion prior to syringe/dosing solution preparation.

Dosing solutions for the 10, 30 and 60 μg dose levels will be prepared from MTRV001 drug product using MTRV001 placebo as the diluent, in accordance with the Pharmacy Manual. For placebo and the 90 μg MTRV001 dose level, no solution preparation is needed, and syringes may be directly prepared from the vialed placebo and MTRV001 drug product, respectively. Prepared syringes of MTRV001 or placebo may be stored at ambient conditions in accordance with the Pharmacy Manual. The administration volume for all dose levels and placebo is 0.5 mL. Each 0.5 mL dose and placebo will contain 0.5 mg aluminum (in the form of aluminum hydroxide).

### Study Interventions Preparation, Handling, Storage, and Accountability

[symbol]

reconciliation, and record maintenance (i.e., receipt, reconciliation, and final disposition records). Each dose of study intervention that is dispensed and used by each participant will be documented.

The Pharmacist, Investigator, or designee must also satisfy regulatory requirements regarding drug accountability. All doses of study intervention will be reconciled and retained or destroyed according to applicable regulations. Additional guidance and information for the final disposition of unused study interventions are provided in the Pharmacy Manual.

Study supplies (MTRV001 drug product and MTRV001 placebo vials) will be sent to the study site in an insulated container with a temperature tracker to ensure no significant deviation (outside the range of 5° C. 3° C.) occurred. Data from the temperature tracker should be downloaded and shared via an email to the relevant email list indicated in the Pharmacy Manual. After receipt of the study supplies, they must be stored in a secure, environmentally controlled area at 5 C°±3° C., and monitored (manual or automated) in accordance with the labeled storage conditions with access limited to the Investigator and authorized site staff.

### Method of Assigning Participants to Treatment Groups

Following the sentinel group of 2 participants in Cohorts 2 through 5 (who will each receive MTRV001 in an open-label manner), the remaining participants in each cohort will be randomized to receive either MTRV001 or placebo.

After confirmation of participant's eligibility and at the last practical moment prior to study intervention administration, participants in the double-blind part of each cohort (Cohorts 2 through 5) will be centrally allocated to either MTRV001 or placebo using an IWRS and per a computer-generated randomization list.

The IWRS will be used to assign unique participant numbers, allocate participants to study intervention group at the randomization visit, and study intervention to participants at each study intervention visit according to the randomization scheme generated by the biostatistician.

The IWRS module is linked to the EDC data capture portion of the clinical database. Once a participant is randomized to the study, all data entry can begin automatically.

### Blinding

Following administration of the open-label MTRV001 to the sentinel group of 2 participants in Cohorts 2 through 5, the remaining participants in the cohort will be randomly assigned to receive either MTRV001 or placebo in a double-blinded manner. In Cohorts 2 through 4, the remaining participants will be randomized (i.e., 10 participants to MTRV001 (18 participants to MTRV001 and 4 participants to placebo). The participant and study staff (other than the unblinded pharmacist) will be blinded to treatment. The pharmacist who will be unblinded, will prepare doses of blinded study intervention according to the randomization scheme generated by the biostatistician.

At the study site, only the pharmacist will have access to the randomization schedule.

If unblinding is deemed necessary by the Investigator and the Sponsor, the unblinded pharmacist will be asked to disclose the study intervention information to the Investigator (and the SMC, as appropriate). The decision and process of unblinding a participant shall be appropriately documented according to the Safety Manual. If unblinding is required, the Sponsor must be notified immediately.

Following the database lock for the preparation of the CSR (which will initially include data up to Visit 6 (Day 57 [±4 days]), the study participants and a subgroup of study personnel responsible for continued conduct of the study (including the Investigator or designee) will remain blinded. The blinded Investigator or designee will assess the relationship of any SAEs or NOCIs reported after Visit 6 (Day 57 [±4 days]).

In the event of a quality assurance audit, the auditor(s) will be allowed access to unblinded study intervention records at the site(s) to verify that randomization/dispensing has been conducted accurately.

### Study Intervention Compliance

When participants are dosed at the site, they will receive study intervention directly from the Investigator or designee, under medical supervision. The date and time of each dose administered at the site will be recorded in the source documents and recorded in the eCRF.

### Concomitant Therapy

### Permitted and Restricted Therapies

Participants who have undergone chronic treatment with immunosuppressant agents or other immune-modifying drugs within 6 months prior to receiving the first dose of study intervention are not eligible for study and these agents are not permitted during the study. Short-term use of corticosteroids (<14 days) for an acute illness are allowed but last dose should be ≥28 days prior to administration of the first dose of study intervention. In addition, the use of topical, inhaled, and nasal glucocorticoids is permitted.

This protocol places no restrictions on rescue medications, and the Investigator will recommend medication for symptomatic relief, if necessary. [symbol] of the investigational product) prior to prior to receiving the first dose of study intervention are not eligible and other investigational products are not permitted during the study.

Participants who have been administered any vaccine within 60 days of receiving the first dose of study intervention are not eligible. Administration of pneumococcal vaccines (other than study intervention) is not permitted until after Visit 7 (Day 210 [±14 days]). Administration of any other vaccine is not permitted until after Visit 6 (Day 57 [±4 days]).

Administration of immunoglobulins and/or any blood products within the 3 months preceding Day 1 or planned administration of such products during the study and up to Visit 6 (Day 57 [±4 days]) is not permitted.

### Record of Concomitant Medication

Participants will be asked about their use of concomitant medications at every study visit. Any medication used within 30 days of administration of the first dose of study intervention through the final study visit (Visit 7 [Day 210 (±14 days)]) will be recorded in the eCRF. This will include all prescription drugs, over-the-counter medications, herbal or other supplements, vitamins, and minerals. The name of each drug along with dates of administration, dose, frequency, and reason for use will be recorded. Participants will also be asked to record concomitant medications used to treat reactogenicity events on the diary cards.

### Possible Drug Interactions

Immunosuppressants and other immune-modifying agents could interfere with the immune response to vaccination and should be avoided.

### Dose Modification

There will be no modification of dose in an individual participant. This study is a dose escalation study, with potential for dose modification by the Investigator and Sponsor between cohorts. If a particular dose level of MTRV001 is judged to be poorly tolerated, an intermediate dose of MTRV001 (i.e., a dose in between the previously tolerated dose and the poorly tolerated dose) may be evaluated, pending further review and concurrence by the SMC and the TRB.

Withdrawal of Participants from the Study or Discontinuation of Study Interventions

### Reasons for Withdrawal/Discontinuation

Participants are free to withdraw from participation in the study at any time upon request. Participants may be withdrawn from the study at any time at the discretion of the Investigator or at the request of the Sponsor.

The reasons for discontinuation from study intervention and reasons for withdrawal from study should be documented in the eCRF. Any of the following may apply:
Noncompliance with the protocol.
A serious or intolerable AE that in the Investigator's opinion requires discontinuation of study intervention.
Laboratory result that reveals a safety concern that in the Investigator's opinion requires discontinuation of study intervention.
Development of an illness not consistent with the protocol requirements or justifies withdrawal.
Other (e.g., pregnancy, development of contraindication to use of study intervention).
Lost to follow-up.
Termination of the study by the Sponsor.

Investigators will follow participants who are withdrawn as a result of an SAE/AE until the event has returned to normal or stabilized, the event is otherwise explained, or the participant is lost to follow-up.

Handling of Withdrawals from Study or Discontinuations from Study Intervention

If a participant withdraws from the study prior to Visit 5 (Day 43 [±4 days]), the site should attempt to complete the assessments listed for an early termination visit. If a participant withdraws from the study prematurely after Visit 5 (Day 43 [±4 days]), the site should attempt to complete Visit 7 (Day 210 [±14 day]) assessments. It is especially important to obtain follow-up data on any ongoing AEs leading to withdrawal or ongoing SAEs.

### Lost to Follow-up

A participant will be considered lost to follow-up if he/she repeatedly fails to return for scheduled visits and is unable to be contacted by the study site.

The following actions will be taken if a participant fails to return to the clinic for a required study visit:

The site must attempt to contact the participant and reschedule the missed visit as soon as possible, counsel the participant on the importance of maintaining the assigned visit schedule and ascertain if the participant wants to or will continue in the study.

Before a participant is deemed “lost to follow-up”, the Investigator or designee will make every effort to regain contact with the participant: 1) where possible, make 3 telephone calls (or preferred form of the participant); 2) if necessary, send a certified letter (or an equivalent local method) to the participant's last known mailing address, and 3) if a participant has given the appropriate consent, contact the participant's general practitioner or caretaker documented in the participant's medical record.

### Replacements

If a participant is withdrawn by the Investigator or withdraws from the study before receiving both doses of the study intervention and is unable to be followed up through Day 29 for reasons other than reactogenicity AEs may be replaced. In the event that an additional participant is enrolled, the IWRS and associated computer-generated randomization list will ensure that unblinding would not occur. Additional participants would receive the same blinded study intervention as the withdrawn participant, as per the randomization scheme.

### Study Assessments and Procedures

### Immunogenicity Assessments

Blood samples will be drawn for immunogenicity assessments at the visits. At Visits 2 (Day 1) and 4 (Day 29 [±2 days]), the samples will be taken prior to administration of study intervention. Additional instructions for collection, storage, and shipment of samples will be provided in the Study Reference Manual. The samples will be analyzed for the following:
Serum IgG for anti-PLY and anti-CbpA. GMTs will be measured by ELISA. In addition, samples will be banked for future exploratory analyses:
In vitro PLY neutralization assay.
In vitro inhibition of CbpA-mediated cell adhesion assay.

Retention time and possible analyses of samples after the end of study are specified in the respective ICF.

### REFERENCES

1. Bologa M, Kamtchoua T, Hopfer R, Sheng X, Hicks B, Bixler G, Hou V, Pehlic V, Yuan T, Gurunathan S. Safety and immunogenicity of pneumococcal protein vaccine candidates: monovalent choline-binding protein A (PcpA) vaccine and bivalent PcpA-pneumococcal histidine triad protein D vaccine. Vaccine. 2012 Dec. 14; 30(52):7461-8. doi: 10.1016/j.vaccine.2012.10.076. Epub 2012 Nov. 2. PMID: 23123106.
2. Brooks W A, Chang U, Sheng X, et al. Safety and immunogenicity of a trivalent recombinant PcpA, PhtD, and PlyD1 pneumococcal protein vaccine in adults, toddlers, and infants: A phase I randomized controlled study. Vaccine. 2015; 33(36):4610-7.
3. Chen A, Mann B, Gao G, et al. Multivalent Pneumococcal Protein Vaccines Comprising Pneumolysoid with Epitopes/Fragments of CbpA and/or PspA Elicit Strong and Broad Protection. Clin Vaccine Immunol. 2015; 22(10):1079-89. [symbol] are essential for cytolytic toxin recognition of cholesterol at the membrane surface. Proc Natl Acad Sci USA. 2010 Mar. 2; 107(9):4341-6. doi: 10.1073/pnas.0911581107. Epub 2010 Feb. 9. PMID: 20145114; PMCID: PMC2840085.
5. Hammitt L L, Campbell J C, Borys D, Weatherholtz R C, Reid R, Goklish N, Moulton L H, Traskine M, Song Y, Swinnen K, Santosham M, O'Brien K L. Efficacy, safety and immunogenicity of a pneumococcal protein-based vaccine co-administered with 13-valent pneumococcal conjugate vaccine against acute otitis media in young children: A phase IIb randomized study. Vaccine. 2019 Dec. 3; 37(51):7482-7492. doi: 10.1016/j.vaccine.2019.09.076. Epub 2019 Oct. 16. PMID: 31629570.
6. Kamtchoua T, Bologa M, Hopfer R, et al. Safety and immunogenicity of the pneumococcal pneumolysin derivative PlyD1 in a single-antigen protein vaccine candidate in adults. Vaccine. 2013; 31(2):327-33.
7. Leroux-Roels G, Maes C, De Boever F, et al. Safety, reactogenicity and immunogenicity of a novel pneumococcal protein-based vaccine in adults: a phase I/II randomized clinical study. Vaccine. 2014; 32(50):6838-46.
8. Luo R, Mann B, Lewis W S, Rowe A, Heath R, Stewart M L, Hamburger A E, Sivakolundu S, Lacy E R, Bjorkman P J, Tuomanen E, Kriwacki R W. Solution structure of choline binding protein A, the major adhesin of Streptococcus pneumoniae. EMBO J. 2005 Jan. 12; 24(1):34-43. doi: 10.1038/sj.emboj.7600490. Epub 2004 Dec. 16. PMID: 15616594; PMCID: PMC544903.
9. Mann B, Thornton J, Heath R, Wade K R, Tweten R K, Gao G, El Kasmi K, Jordan J B, Mitrea D M, Kriwacki R, Maisonneuve J, Alderson M, Tuomanen E L. Broadly protective protein-based pneumococcal vaccine composed of pneumolysin toxoid-CbpA peptide recombinant fusion protein. J Infect Dis. 2014 Apr. 1; 209(7):1116-25. doi: 10.1093/infdis/jit502. Epub 2013 Sep. 16. PMID: 24041791; PMCID: PMC3952665.
10. Odutola A, Ota M O C, Antonio M, Ogundare E O, Saidu Y, Foster-Nyarko E, Owiafe P K, Ceesay F, Worwui A, Idoko O T, Owolabi O, Bojang A, Jarju S, Drammeh I, Kampmann B, Greenwood B M, Alderson M, Traskine M, Devos N, Schoonbroodt S, Swinnen K, Verlant V, Dobbelaere K, Borys D. Efficacy of a novel, protein-based pneumococcal vaccine against nasopharyngeal carriage of Streptococcus pneumoniae in infants: A phase 2, randomized, controlled, observer-blind study. Vaccine. 2017 May 2; 35(19):2531-2542. doi: 10.1016/j.vaccine.2017.03.071. Epub 2017 Apr. 4. PMID: 28389097. [symbol] engineering yields pneumolysin mutants suitable for vaccination against pneumococcal disease. J Biol Chem. 2011 Apr. 8; 286(14):12133-40. doi: 10.1074/jbc.M110.191148. Epub 2011 Feb. 4. PMID: 21296887; PMCID: PMC3069417.
12. Prymula R, Pazdiora P, Traskine M, Ruggeberg J U, Borys D. Safety and immunogenicity of an investigational vaccine containing two common pneumococcal proteins in toddlers: a phase II randomized clinical trial. Vaccine. 2014 May 23; 32(25):3025-34. doi: 10.1016/j.vaccine.2014.03.066. Epub 2014 Apr. 1. PMID: 24699466.
13. Thanawastien A, Joyce K E, Cartee R T, et al. Preclinical in vitro and in vivo profile of a highly attenuated, broadly efficacious pneumolysin genetic toxoid. Vaccine. 2021; 39(11):1652 60.
14. World Health Organization. Guidelines on the nonclinical evaluation of vaccines adjuvants and adjuvanted vaccines. WHO Expert Committee on Biological Standardization. Sixty-fourth report. WHO Technical Report Series No. 987, 2014.
15. World Health Organization. WHO guidelines of nonclinical evaluation of vaccines. Annex I of WHO Technical Report Series, No 927, 2005.
Bonten M J, Huijts S M, Bolkenbaas M, et al. Polysaccharide conjugate vaccine against pneumococcal pneumonia in adults. N Engl J Med. 2015; 372(12):1114-25.
Brandileone M C, Almeida S C G, Minamisava R, Andrade A L. Distribution of invasive Streptococcus pneumoniae serotypes before and 5 years after the introduction of 10-valent pneumococcal conjugate vaccine in Brazil. Vaccine. 2018; 36:2559-66.
CDC, Centers for Disease Control and Prevention. Active Bacterial Core Surveillance Report, Emerging Infections Program Network, Streptococcus pneumoniae. 2017. Available at: https://www.cdc.gov/abcs/reports-findings/survreports/spneul7.html.
CDC, Centers for Disease Control and Prevention. Global pneumococcal disease and vaccine. Updated Nov. 5, 2018. Available at: https://www.cdc.gov/pneumococcal/global.html.
Chen A, Mann B, Gao G, et al. Multivalent Pneumococcal Protein Vaccines Comprising Pneumolysoid with Epitopes/Fragments of CbpA and/or PspA Elicit Strong and Broad Protection. Clin Vaccine Immunol. 2015; 22(10):1079-89.
Daniels C C, Rogers P D, Shelton C M. A review of pneumococcal vaccines: current polysaccharide vaccine recommendations and future protein antigens. J Pediatr Pharmacol Ther. 2016; 21(1):27-35.
ECDC, European Centre for Disease Prevention and Control. Invasive pneumococcal disease. In: ECDC. Annual epidemiological report for 2017. Stockholm: ECDC; 2019. [symbol] induced by the 13-valent pneumococcal conjugate followed by the 23-valent polysaccharide vaccine in HIV-infected adults. J Infect Dis. 2018; 218(1):26-34.
Geno K A, et al. Pneumococcal capsules and their types: past, present, and future. Clin Microbiol Rev. 2015; 28: 871-99.
GBD, Global Burden of Disease 2016 Lower Respiratory Infections Collaborators. Estimates of the global, regional, and national morbidity, mortality, and aetiologies of lower respiratory infections in 195 countries, 1990-2016: a systematic analysis for the Global Burden of Disease Study 2016. Lancet Infect Dis. 2018; 18(11):1191-210.
Goldblatt D, Assari T, Snapper C. The Immunology of Polysaccharide and Conjugate Vaccines. In: Siber G, et al (ed). Pneumococcal Vaccines; The Impact of Conjugate Vaccine. ASM Press, Washington, D C, 2008.
Kamtchoua T, Bologa M, Hopfer R, et al. Safety and immunogenicity of the pneumococcal pneumolysin derivative PlyD1 in a single-antigen protein vaccine candidate in adults. Vaccine. 2013; 31(2):327-33.
Kim L, McGhee S, Tomczyk B, Beall B. Biological and epidemiological features of antibiotic-resistant Streptococcus pneumoniae in pre- and post-conjugate vaccine eras; a United States perspective. Clin Microbiol Rev. 2016; 29:525-52.
Klugman K P, Koornhof H J, Kuhnle V. Clinical and nasopharyngeal isolates of unusual multiply resistant pneumococci. Am J Dis Child. 1986; 140:1186-90.
Koornhof H J, Wasa A, Klugman K. Antimicrobial resistance in Streptococcus pneumoniae: a South African perspective. Clin Infect Dis. 1992; 15:84-90.
Ladhani S N, et al. Rapid increase in non-vaccine serotypes causing invasive pneumococcal disease in England and Wales, 2000-17: a prospective national observational cohort study. Lancet Infect Dis. 2018; 18:441-51.
Lagousi T, Basdeki P, Routsias J, Spoulou V. Novel protein-based pneumococcal vaccines: assessing the use of distinct protein fragments instead of full-length proteins as vaccine Antigens. Vaccines (Basel). 2019; 7.
Leroux-Roels G, Maes C, De Boever F, et al. Safety, reactogenicity and immunogenicity of a novel pneumococcal protein-based vaccine in adults: a phase I/II randomized clinical study. Vaccine. 2014; 32(50):6838-46.
Lewnard J A, Hanage W P. Making sense of differences in pneumococcal serotype replacement. Lancet Infect Dis. 2019; 19:e213-e220. [symbol] pneumolysin mutants suitable for vaccination against pneumococcal disease. J Biol Chem. 2011; 286(14):12133-40.
Orami T, Ford R, Kirkham L A, et al. Pneumococcal conjugate vaccine primes mucosal immune responses to pneumococcal polysaccharide vaccine booster in Papua New Guinean children. Vaccine. 2020; 38(50):7977-88.
Perez J L, Linares J, Bosch M, Lopez de Goicoechea M J, Martin R. Antibiotic resistance of Streptococcus pneumoniae in childhood carriers. J Antimicrob Chemother. 1987; 19:278-80.
Posfay-Barbe K M, Galetto-Lacour A, Ochs M M, et al. Immunity to pneumococcal surface proteins in children with community-acquired pneumonia: a distinct pattern of response to pneumococcal choline-binding protein A. Clin Microbiol Infect. 2011; 17(8):1232-8.
Probe K, Tashani M, Ridda I, Rashid H, Wong M, Booy R. Carrier priming or suppression: understanding carrier priming enhancement of anti-polysaccharide antibody response to conjugate vaccines. Vaccine. 2014; 32(13):1423-30.
Quin L R, Moore Q C, McDaniel L S. Pneumolysin, PspA, and PspC contribute to pneumococcal evasion of early innate immune responses during bacteremia in mice. Infect Immun. 2007; 75:2067-70.
Singleton R J, et al. Invasive pneumococcal disease caused by nonvaccine serotypes among Alaska native children with high levels of 7-valent pneumococcal conjugate vaccine coverage. Jama. 2007; 297:1784-92.
The Global Pneumococcal Sequencing Consortium (GPSC). Available at: https://www.pneumogen.net/gps/serotypes.html [accessed on 15 Sep. 2022].
U.S. Department of Health and Human Services FDA CBER. Guidance for Industry: Toxicity Grading Scale for Healthy Adult and Adolescent Volunteers Enrolled in Preventive Vaccine Clinical Trials. September 2007.
van den Biggelaar A H J, Pomat W S, Masiria G, et al. Immunogenicity and immune memory after a pneumococcal polysaccharide vaccine booster in a high-risk population primed with 10-valent or 13-valent pneumococcal conjugate vaccine: a randomized controlled trial in Papua New Guinean children. Vaccines (Basel). 2019; 7(1):17. Published 2019 Feb. 4.
WHO World Health Organization. Pneumococcus, 2018. Available at: https://www.who.int/immunization/monitoring_surveillance/burden/vpd/WHO_Surveillance VaccinePreventable_17_Pneumococcus_R2.pdf?ua=1.

### VI. EMBODIMENTS

Embodiment 1. A polypeptide comprising an amino acid sequence of SEQ ID NO: 43. [symbol] glycosylated.

Embodiment 3. The polypeptide of embodiment 1, wherein the polypeptide is not glycosylated.

Embodiment 4. A nucleic acid sequence encoding the polypeptide of any one of embodiments 1-3.

Embodiment 5. A vector comprising the nucleic acid sequence of embodiment 4.

Embodiment 6. A composition comprising the polypeptide of any one of embodiments 1-3 and a pharmaceutically acceptable carrier.

Embodiment 7. A composition comprising the polypeptide of any one of embodiments 1-3, further comprising an adjuvant.

Embodiment 8. The composition of embodiment 7, wherein the adjuvant comprises an aluminum salt, an oil-in-water emulsion, a saponin, complete Freund's adjuvant, incomplete Freund's adjuvant, a cytokine, monophosphoryl lipid A (MPL), 3-O-deacylated MPL (3dMPL), QS21, a polyoxyethylene ether, a polyoxyethylene ester, a polyoxyethylene sorbitan ester surfactant, an octoxynol, a polyoxyethylene alkyl ether, a ester surfactant, an immunostimulatory oligonucleotide, an immunostimulant, a particle of metal salt, IM2, a sterol, an immunostimulating agent, a N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE or any combination thereof.

Embodiment 9. The composition of embodiment 7, wherein the adjuvant comprises aluminum hydroxide, aluminum phosphate or aluminum sulfate.

Embodiment 10. The composition of embodiment 7, wherein the adjuvant comprises aluminum hydroxide.

Embodiment 11. The composition of embodiment 7, wherein the adjuvant comprises Alhydrogel®.

Embodiment 12. The composition of any one of embodiments 1-11, further comprising a pharmaceutically acceptable carrier.

Embodiment 13. A composition comprising a purified polypeptide comprising an amino acid sequence of SEQ ID NO: 43, wherein the composition contains less than 8% of one or more contaminants.

Embodiment 14. The composition of embodiment 13, wherein the composition contains less than 5% of one or more contaminants. [symbol] less than 1% of one or more contaminants.

Embodiment 16. The composition of any one of embodiments 13-15, wherein the contaminant comprises a host cell protein.

Embodiment 17. A composition comprising a purified polypeptide comprising an amino acid sequence of SEQ ID NO: 43 and an adjuvant, wherein the composition contains less than 8% of one or more contaminants.

Embodiment 18. The composition of embodiment 17, wherein the composition contains less than 5% of one or more contaminants.

Embodiment 19. The composition of embodiment 17, wherein the composition contains less than 1% of one or more contaminants.

Embodiment 20. The composition of any one of embodiments 17-19, wherein the contaminant comprises a host cell protein.

Embodiment 21. The composition of any one of embodiments 17-20, wherein the adjuvant comprises an aluminum hydroxide.

Embodiment 22. The composition of embodiment 21, wherein the aluminum hydroxide is Alhydrogel®.

Embodiment 23. An injectable formulation comprising a polypeptide comprising an amino acid sequence of SEQ ID NO: 43, a buffer, a salt and a surfactant.

Embodiment 24. The injectable formulation of embodiment 23, wherein i) the polypeptide is at a concentration of about 0.5 mg/mL to about 1.5 mg/mL; ii) the buffer is at a concentration of about 5 mM to about 20 mM; iii) the salt is at a concentration of about 50 mM to about 200 mM; and iv) the surfactant is at a concentration of about 175 μg/mL to about 375 μg/mL; and wherein the pH level of the formulation is between pH 6 and pH 9.

Embodiment 25. The injectable formulation of embodiment 24, wherein the polypeptide is at a concentration of about 0.5 mg/mL to about 1.5 mg/mL, the buffer is at a concentration of about 10 mM, the salt is at a concentration of about 154 mM and the surfactant is at a concentration of about 275 μg/mL, and wherein the pH level of the formulation is about 7.4.

Embodiment 26. The injectable formulation of any one of embodiments 23-25, wherein the buffer is a sodium phosphate buffer.

Embodiment 27. The injectable formulation of any one of embodiments 23-26, wherein the salt is sodium chloride (NaCl). [symbol] the surfactant is Tween 20.

Embodiment 29. An injectable formulation comprising a polypeptide comprising an amino acid sequence of SEQ ID NO: 43, a buffer, a salt, a surfactant and an adjuvant.

Embodiment 30. The injectable formulation of embodiment 29, wherein i) the polypeptide is at a concentration of about 2 μg/mL to about 300 μg/mL; ii) the buffer is at a concentration of about 5 mM to about 15 mM; iii) the salt is at a concentration is at about 130 mM to about 150 mM; iv) the surfactant is at a concentration is at about 2 μg/mL to about 100 μg/mL; v) the adjuvant is at a concentration of about 0.01 mg/mL to about 3 mg/mL; and wherein the pH level of the formulation is between pH 6 and pH 9.

Embodiment 31. The injectable formulation of any one of embodiments 29-30, wherein the buffer is a phosphate buffer.

Embodiment 32. The injectable formulation of any one of embodiments 29-31, wherein the salt is sodium chloride (NaCl).

Embodiment 33. The injectable formulation of any one of embodiments 29-32, wherein the surfactant is Tween 20.

Embodiment 34. The injectable formulation of any one of embodiments 29-33, wherein the adjuvant is Alhydrogel®.

Embodiment 35. The injectable formulation of embodiment 34, wherein the phosphate buffer is a concentration of about 9 mM, the NaCl is at a concentration of about 138.6 mM and the Alhydrogel® is at a concentration of about 1 mg/mL.

Embodiment 36. The injectable formulation of embodiment 35, wherein the polypeptide is at a concentration of about 10 μg/mL to about 30 μg/mL and wherein the Tween20 is at a concentration of about 4 μg/mL to about 8 μg/mL.

Embodiment 37. The injectable formulation of embodiment 36, wherein the polypeptide is at a concentration of about 20 μg/mL.

Embodiment 38. The injectable formulation of embodiment 35, wherein the polypeptide is at a concentration of about 48 μg/mL to about 72 μg/mL and wherein the Tween20 is at a concentration of about 12 μg/mL to about 24 μg/mL.

Embodiment 39. The injectable formulation of embodiment 38, wherein the polypeptide is at a concentration of about 60 μg/mL.

Embodiment 40. The injectable formulation of embodiment 24-26, wherein the polypeptide is at a concentration of about 96 μg/mL to about 144 μg/mL and wherein the Tween20 is at a concentration of about 23 μg/mL to about 38 μg/mL. [symbol] polypeptide is at a concentration of about 120 μg/mL.

Embodiment 42. The injectable formulation of embodiment 24-26, wherein the polypeptide is at a concentration of about 144 μg/mL to about 216 μg/mL and wherein the Tween20 is at a concentration of about 35 μg/mL to about 73 μg/mL.

Embodiment 43. The injectable formulation of embodiment 33, wherein the polypeptide is at a concentration of about 180 μg/mL.

Embodiment 44. A method of treating, prophylactically preventing, or reducing the occurrence of a condition, disease, or infection caused by Streptococcus pneumoniae, in a subject in need thereof comprising administering to the subject at least one dose of the composition of embodiments 1-3 and 6-22 or the injectable formulation of embodiments 23-43.

Embodiment 45. The method of embodiment 35, wherein the subject in need thereof is administered with no more than five doses, no more than four doses, no more than three doses or no more than two doses.

Embodiment 46. The method of embodiment 45, wherein the subject in need thereof is administered with no more than two doses.

Embodiment 47. The method of any one of embodiments 35-36, wherein a dose comprises about 5 μg to about 110 μg of the polypeptide.

Embodiment 48. The method of embodiment 47, wherein a dose comprises about 10 μg of the polypeptide.

Embodiment 49. The method of embodiment 47, wherein a dose comprises about 30 μg of the polypeptide.

Embodiment 50. The method of embodiment 47, wherein a dose comprises about 60 μg of the polypeptide.

Embodiment 51. The method of embodiment 47, wherein a dose comprises about 90 μg of the polypeptide.

Embodiment 52. The method of any one of embodiments 44-51, wherein the composition or the injectable formulation is administered intramuscularly.

Embodiment 53. A method of producing a recombinant polypeptide comprising an amino acid sequence of SEQ ID NO: 43 in a host cell.

Embodiment 54. The method of embodiment 53, wherein the method comprises: a) providing a vector comprising a nucleic acid encoding the polypeptide; b) introducing the vector into a population of host cells; c) culturing the population of host cells under conditions that allow for the expression of the polypeptide; d) disrupting the cell membranes of the host sequence of SEQ ID NO: 43.

Embodiment 55. The method of embodiment 54, wherein the host cell is an E. coli cell.

Embodiment 56. The method of any one of embodiments 53-55, further comprising at least one purification step.

Embodiment 57. The method of embodiment 56, wherein the purification step is hydrophobic interaction chromatography, anion exchange chromatography, cation exchange chromatography, hydroxyapatite chromatography, gel filtration chromatography, size exclusion chromatography, hydrophilic interaction chromatography or a combination thereof.

Embodiment 58. The method of embodiment 57, wherein the purification step is hydrophobic interaction chromatography.

Embodiment 59. The method of embodiment 57, wherein the purification step is anion exchange chromatography.

Embodiment 60. The method of embodiment any one of embodiments 53-55, further comprising: f) contacting the polypeptide with a first separation means; g) eluting the polypeptide from the first separation means under conditions that allow for preferential detachment of the polypeptide; h) contacting the eluted polypeptide with a second separation means; and i) eluting the polypeptide from the second separation means under conditions that allow for preferential detachment of the polypeptide; and wherein the first separation means and the second separations means are not the same.

Embodiment 61. The method of embodiment 60, wherein the first separation means is a hydrophobic interaction chromatography resin or an anion exchange chromatography resin.

Embodiment 62. The method of any one of embodiments 60-61, wherein the second separation means is a hydrophobic interaction chromatography resin or an anion exchange chromatography resin.

Embodiment 63. The method of any one of embodiments 60-62, further comprising: h) contacting the eluted polypeptide with a 0.2 μm filter.

Embodiment 64. A composition comprising the polypeptide produced by the method of any one of embodiments 53-63.

Embodiment 65. The composition of embodiment 64, wherein the composition comprises less than 1.0% host cell protein.

1. An immunogenic fusion protein comprising an amino acid sequence of SEQ ID NO: 43.

2. A polynucleotide encoding the immunogenic fusion protein of claim 1.

3. A host cell, comprising the polynucleotide of claim 2.

4. A composition comprising the immunogenic fusion protein of claim 1 and a pharmaceutically acceptable carrier.

5. The composition of claim 4, wherein the immunogenic fusion protein is glycosylated.

6. The composition of claim 4, wherein the immunogenic fusion protein is not glycosylated.

7. The composition of any one of claims 4-6, further comprising at least one adjuvant.

8. The composition of claim 7, wherein the adjuvant comprises aluminum hydroxide, aluminum phosphate or aluminum sulfate.

9. The composition of claim 8, wherein the adjuvant comprises aluminum hydroxide.

10. The composition of claim 9, wherein the aluminum hydroxide comprises Alhydrogel®.

11. A composition comprising:
i) a population of purified immunogenic fusion proteins, wherein at least about 90% of the purified immunogenic fusion proteins are full-length purified immunogenic fusion proteins comprising the amino acid sequence of SEQ ID NO: 43;
ii) less than 80,000 ng of host cell protein/mg of purified immunogenic fusion protein; and/or
iii) less than 17 EU of endotoxin/mg of purified immunogenic fusion protein.

12. The composition of claim 11, wherein the composition comprises:
i) a population of purified immunogenic fusion proteins, wherein about 95%, about 96%, about 97%, about 98% or about 99% of the purified immunogenic fusion proteins are full-length purified immunogenic fusion proteins comprising the amino acid sequence of SEQ ID NO: 43;
ii) less than 50 ng of host cell protein/mg of purified immunogenic fusion protein; and/or
iii) less than 2 EU of endotoxin/mg of purified immunogenic fusion protein.

13. A method of producing an immunogenic fusion protein, comprising the steps of:
a) culturing a population of the host cells expressing an immunogenic fusion protein comprising the amino acid sequence of SEQ ID NO: 43 in a condition suitable for the population of host cells to produce the immunogenic fusion protein;
b) disrupting the cell membranes of the host cells;
c) recovering a sample comprising the immunogenic fusion protein and one or more impurities;
d) contacting the sample comprising the immunogenic fusion protein with a hydrophobic interaction chromatography resin and eluting the immunogenic fusion protein from the hydrophobic interaction chromatography resin under conditions that allow for preferential detachment of the immunogenic fusion protein, thereby obtaining an eluate comprising the immunogenic fusion protein;
e) subjecting the eluate comprising the immunogenic fusion protein of step d) to a flow through anion exchange resin, thereby obtaining an eluate comprising the immunogenic fusion protein; and
f) contacting the eluate comprising the immunogenic fusion protein of step e) with a multi-modal chromatography resin and eluting the immunogenic fusion protein from the multi-modal chromatography resin under conditions that allow for preferential detachment of the immunogenic fusion protein, thereby obtaining an eluate comprising the immunogenic fusion protein.

14. The method of claim 13, further comprising the step of:
g) contacting the eluate comprising the immunogenic fusion protein of step f) with a flow through anion exchange membrane; thereby obtaining an eluate comprising the immunogenic fusion protein.

15. The method of claim 14, further comprising the steps of:
h) contacting the eluate comprising the immunogenic fusion protein of step g) with an ultrafiltration/diafiltration membrane; and
i) washing the immunogenic fusion protein from the ultrafiltration/diafiltration membrane under conditions that allow for preferential detachment of the immunogenic fusion protein, thereby obtaining an eluate comprising the immunogenic fusion protein.

16. The method of claim 15, further comprising the step of:
j) contacting the eluate comprising the immunogenic fusion protein of step i) with a 0.2 μm filter.

17. The method of any one of claims 13-16, wherein the host cell is an E. coli cell.

18. A composition comprising a purified immunogenic fusion protein produced by the method of any one of claims 13-17.

19. A formulation comprising:
i) an immunogenic fusion protein comprising the amino acid sequence of SEQ ID NO: 43;
ii) a surfactant;
iii) a buffer; and
iv) a salt.

20. The formulation of claim 19, wherein the surfactant is at a concentration of about 175 μg/mL to about 375 μg/mL.

21. The formulation of claim 19, wherein
i) the immunogenic fusion protein is at a concentration of about 0.5 mg/mL to about 1.5 mg/mL;
ii) the surfactant is at a concentration of about 175 μg/mL to about 375 μg/mL;
iii) the buffer is at a concentration of about 5 mM to about 20 mM;
iv) the salt is at a concentration of about 50 mM to about 200 mM; and
wherein the pH level of the formulation is between pH 6 and pH 9.
i) the immunogenic fusion protein is at a concentration of about 0.8 mg/mL to about 1.2 mg/mL;
ii) the surfactant is at a concentration of about 275 μg/mL;
iii) the buffer is at a concentration of about 10 mM;
iv) the salt is at a concentration of about 154 mM; and
wherein the pH level of the formulation is about 7.4.

23. The formulation of any one of claims 19-22, wherein the buffer comprises sodium phosphate, the salt comprises sodium chloride (NaCl) and/or the surfactant comprises polysorbate 20.

24. The formulation of any one of claims 19-23, further comprising an adjuvant.

25. The formulation of claim 24, wherein the adjuvant is selected from the group consisting of aluminum hydroxide, aluminum phosphate and aluminum sulfate; and wherein the adjuvant is at a concentration of about 0.5 mg/mL to about 2 mg/mL.

26. The formulation of claim 25, wherein the adjuvant is at a concentration of about 1 mg/mL.

27. The formulation of any one of claims 24-26, wherein the adjuvant is aluminum hydroxide.

28. The formulation of claim 27, wherein the aluminum hydroxide is Alhydrogel®.

29. A formulation comprising:
about 1.0 mg/mL of an immunogenic fusion protein comprising the amino acid sequence of SEQ ID NO: 43, about 275 μg/mL polysorbate 20, about 10 mM sodium phosphate and about 154 mM sodium chloride, and wherein the pH level of the formulation is about 7.4.

30. A formulation comprising:
about 20 μg/mL of an immunogenic fusion protein comprising the amino acid sequence of SEQ ID NO: 43, about 275 μg/mL polysorbate 20, about 1 mg/mL of aluminum hydroxide in the formulation is about 7.4.

31. A formulation comprising:
about 60 μg/mL of an immunogenic fusion protein comprising the amino acid sequence of SEQ ID NO: 43, about 275 μg/mL polysorbate 20, about 1 mg/mL of aluminum hydroxide in 9 mM of sodium phosphate and about 139 mM sodium chloride, and wherein the pH level of the formulation is about 7.4.

32. A formulation comprising:
about 120 μg/mL of an immunogenic fusion protein comprising the amino acid sequence of SEQ ID NO: 43, about 275 μg/mL polysorbate 20, about 1 mg/mL of aluminum hydroxide in 9 mM of sodium phosphate and about 139 mM sodium chloride, and wherein the pH level of the formulation is about 7.4.

33. A formulation comprising:
about 180 μg/mL of an immunogenic fusion protein comprising the amino acid sequence of SEQ ID NO: 43, about 275 μg/mL polysorbate 20, about 1 mg/mL of aluminum hydroxide in 9 mM of sodium phosphate and about 139 mM sodium chloride, and wherein the pH level of the formulation is about 7.4.

34. A method of inducing a protective immune response in a subject comprising administering to the subject the composition of any one of claims 4-12 or 18 or the formulation of any one of claims 19-33.

35. A method of immunizing a subject against an infection caused by Streptococcus pneumoniae, the method comprising administering to the subject the composition of any one of claims 4-12 or 18 or the formulation of any one of claims 19-33.

36. A method of treating, prophylactically preventing, or reducing the occurrence of a condition, disease, or infection caused by Streptococcus pneumoniae, in a subject in need thereof comprising administering to the subject the composition of any one of claims 4-12 or 18 or the formulation of any one of claims 19-33. [symbol] least one dose of the immunogenic fusion protein.

38. The method of claim 37, wherein the subject is administered with no more than two doses of the immunogenic fusion protein.

39. The method of any one of claims 37-38, wherein the dose further comprises about 1 mg/mL of aluminum hydroxide.

40. The method of any one of claims 37-39, wherein the dose comprises about 1 μg to about 150 μg of the immunogenic fusion protein.

41. The method of claim 40, wherein the dose comprises about 10 μg of the immunogenic fusion protein.

42. The method of claim 40, wherein the dose comprises about 30 μg of the immunogenic fusion protein.

43. The method of claim 40, wherein the dose comprises about 60 μg of the immunogenic fusion protein.

44. The method of claim 40, wherein the dose comprises about 90 μg of the immunogenic fusion protein.

45. The method of any one of claims 37-44, wherein the amount of time between each dose is from about four weeks to about one year.

46. The method of claim 45, wherein the amount of time between each dose is about one week, about two weeks, about three weeks or about four weeks.

47. The method of claim 46, wherein the amount of time between each dose is about four weeks. [symbol] is administered by parenteral administration.

49. The method of any one of claim 48, wherein the parenteral administration is by intramuscular injection.

50. The method of any one of claims 34-49, wherein the subject is between 0 and 80 years of age.

51. The method of claim 50, wherein the subject is between 0 and 2 years of age.

52. The method of claim 50, wherein the subject is between 18 and 50 years of age.

53. The method of claim 50, wherein the subject is between 60 and 75 years of age.


SEQUENCE LISTING (43 sequences)

SEQ ID NO: 1 - protein, 30 aa, Streptococcus pneumoniae
AVKKAEDQKEEDRRNYPTNTYKTLELEKAE

SEQ ID NO: 2 - protein, 17 aa, Streptococcus pneumoniae
KEKAKEPRNEEKVKQVK

SEQ ID NO: 3 - protein, 30 aa, synthetic construct
  REGION 1..30: note = Cysteine mutant of R21 fragment of Streptococcus pneumoniae
ACKKAEDQKEEDRRNYPTNTYKTLELECAE

SEQ ID NO: 4 - protein, 17 aa, synthetic construct
  REGION 1..17: note = Cysteine mutant of R22 fragment of Streptococcus pneumoniae
KECAKEPRNEEKVKQCK

SEQ ID NO: 5 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 6 - DNA, 1413 bp, Streptococcus pneumoniae
  source 1..1413: mol_type = genomic DNA
atggcaaataaagcagtaaatgactttatactagctatgaattacgataaaaagaaactcttgacccatcagggagaaagtattgaaaatcgtttcatcaaagagggtaatcagctacccgatgagtttgttgttatcgaaagaaagaagcggagcttgtcgacaaatacaagtgatatttctgtaacagctaccaacgacagtcgcctctatcctggagcacttctcgtagtggatgagaccttgttagagaataatcccactcttcttgcggttgatcgtgctccgatgacttatagtattgatttgcctggtttggcaagtagcgatagctttctccaagtggaagaccccagcaattcaagtgttcgcggagcggtaaacgatttgttggctaagtggcatcaagattatggtcaggtcaataatgtcccagctagaatgcagtatgaaaaaataacggctcacagcatggaacaactcaaggtcaagtttggttctgactttgaaaagacagggaattctcttgatattgattttaactctgtccattcaggtgaaaagcagattcagattgttaattttaagcagatttattatacagtcagcgtagacgctgttaaaaatccaggagatgtgtttcaagatactgtaacggtagaggatttaaaacagagaggaatttctgcagagcgtcctttggtctatatttcgagtgttgcttatgggcgccaagtctatctcaagttggaaaccacgagtaagagtgatgaagtagaggctgcttttgaagctttgataaaaggagtcaaggtagctcctcagacagagtggaagcagattttggacaatacagaagtgaaggcggttattttagggggcgacccaagttcgggtgcccgagttgtaacaggcaaggtggatatggtagaggacttgattcaagaaggcagtcgctttacagcagatcatccaggcttgccgatttcctatacaacttcttttttacgtgacaatgtagttgcgacctttcaaaacagtacagactatgttgagactaaggttacagcttacagaaacggagatttactgctggatcatagtggtgcctatgttgcccaatattatattacttgggatgaattatcctatgatcatcaaggtaaggaagtcttgactcctaaggcttgggacagaaatgggcaggatttgacggctcactttaccactagtattcctttaaaagggaatgttcgtaatctctctgtcaaaattagagagtgtaccgggcttgcctgggaatggtggcgtacggtttatgaaaaaaccgatttgccactagtgcgtaagcggacgatttctatttggggaacaactctctatcctcaggtagaggataaggtagaaaatgac

SEQ ID NO: 7 - protein, 471 aa, synthetic construct
  REGION 1..471: note = L460D pneumolysin mutant
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTDYPQVEDKVEND

SEQ ID NO: 8 - protein, 469 aa, synthetic construct
  REGION 1..469: note = Delta6N385 pneumolysin mutant
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 9 - protein, 520 aa, synthetic construct
  REGION 1..520: note = YPT-L460D-NEEK fusion protein
MACKKAEDQKEEDRRNYPTNTYKTLELECAEGGANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTDYPQVEDKVENDKECAKEPRNEEKVKQCK

SEQ ID NO: 10 - DNA, 1566 bp, synthetic construct
  misc_feature 1..1566: note = YPT-L460D-NEEK fusion protein nucleotide sequence
  source 1..1566: mol_type = other DNA
catatggcttgtaaaaaagccgaggatcaaaaagaagaagatcgccgtaactacccaaccaatacttacaaaacgcttgaacttgaatgtgctgagggtggtgcaaataaagcagtaaatgactttatactagctatgaattacgataaaaagaaactcttgacccatcagggagaaagtattgaaaatcgtttcatcaaagagggtaatcagctacccgatgagtttgttgttatcgaaagaaagaagcggagcttgtcgacaaatacaagtgatatttctgtaacagctaccaacgacagtcgcctctatcctggagcacttctcgtagtggatgagaccttgttagagaataatcccactcttcttgcggttgatcgtgctccgatgacttatagtattgatttgcctggtttggcaagtagcgatagctttctccaagtggaagaccccagcaattcaagtgttcgcggagcggtaaacgatttgttggctaagtggcatcaagattatggtcaggtcaataatgtcccagctagaatgcagtatgaaaaaataacggctcacagcatggaacaactcaaggtcaagtttggttctgactttgaaaagacagggaattctcttgatattgattttaactctgtccattcaggtgaaaagcagattcagattgttaattttaagcagatttattatacagtcagcgtagacgctgttaaaaatccaggagatgtgtttcaagatactgtaacggtagaggatttaaaacagagaggaatttctgcagagcgtcctttggtctatatttcgagtgttgcttatgggcgccaagtctatctcaagttggaaaccacgagtaagagtgatgaagtagaggctgcttttgaagctttgataaaaggagtcaaggtagctcctcagacagagtggaagcagattttggacaatacagaagtgaaggcggttattttagggggcgacccaagttcgggtgcccgagttgtaacaggcaaggtggatatggtagaggacttgattcaagaaggcagtcgctttacagcagatcatccaggcttgccgatttcctatacaacttcttttttacgtgacaatgtagttgcgacctttcaaaacagtacagactatgttgagactaaggttacagcttacagaaacggagatttactgctggatcatagtggtgcctatgttgcccaatattatattacttgggatgaattatcctatgatcatcaaggtaaggaagtcttgactcctaaggcttgggacagaaatgggcaggatttgacggctcactttaccactagtattcctttaaaagggaatgttcgtaatctctctgtcaaaattagagagtgtaccgggcttgcctgggaatggtggcgtacggtttatgaaaaaaccgatttgccactagtgcgtaagcggacgatttctatttggggaacaactgactatcctcaggtagaggataaggtagaaaatgacaaagagtgtgctaaggaacctcgaaacgaggaaaaagttaagcaatgtaaatag

SEQ ID NO: 11 - protein, 518 aa, synthetic construct
  REGION 1..518: note = YPT-Delta6N385-NEEK fusion protein
MACKKAEDQKEEDRRNYPTNTYKTLELECAEGGANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIRECTGLAWEWWRTVYEKTDLPLVRKRTISIWGTTLYPQVEDKVENDKECAKEPRNEEKVKQCK

SEQ ID NO: 12 - protein, 693 aa, Streptococcus pneumoniae
MFASKSERKVHYSIRKFSVGVASVVVASLVMGSVVHATENEGATQVPTSSNRANESQAEQGEQPKKLDSERDKARKEVEEYVKKIVGESYAKSTKKRHTITVALVNELNNIKNEYLNKIVESTSESQLQILMMESRSKVDEAVSKFEKDSSSSSSSDSSTKPEASDTAKPNKPTEPGEKVAEAKKKVEEAEKKAKDQKEEDRRNYPTITYKTLELEIAESDVEVKKAELELVKVKANEPRDEQKIKQAEAEVESKQAEATRLKKIKTDREEAEEEAKRRADAKEQGKPKGRAKRGVPGELATPDKKENDAKSSDSSVGEETLPSPSLKPEKKVAEAEKKVEEAKKKAEDQKEEDRRNYPTNTYKTLELEIAESDVEVKKAELELVKEEAKEPRNEEKVKQAKAEVESKKAEATRLEKIKTDRKKAEEEAKRKAAEEDKVKEKPAEQPQPAPAPKAEKPAPAPKPENPAEQPKAEKPADQQAEEDYARRSEEEYNRLTQQQPPKTEKPAQPSTPKTGWKQENGMWYFYNTDGSMATGWLQNNGSWYYLNSNGAMATGWLQNNGSWYYLNANGSMATGWLQNNGSWYYLNANGSMATGWLQYNGSWYYLNANGSMATGWLQYNGSWYYLNANGDMATGWVKDGDTWYYLEASGAMKASQWFKVSDKWYYVNGSGALAVNTTVDGYGVNANGEWVN

SEQ ID NO: 13 - protein, 472 aa, Streptococcus pneumoniae
ENEGATQVPTSSNRANESQAEQGEQPKKLDSERDKARKEVEEYVKKIVGESYAKSTKKRHTITVALVNELNNIKNEYLNKIVESTSESQLQILMMESRSKVDEAVSKFEKDSSSSSSSDSSTKPEASDTAKPNKPTEPGEKVAEAKKKVEEAEKKAKDQKEEDRRNYPTITYKTLELEIAESDVEVKKAELELVKVKANEPRDEQKIKQAEAEVESKQAEATRLKKIKTDREEAEEEAKRRADAKEQGKPKGRAKRGVPGELATPDKKENDAKSSDSSVGEETLPSPSLKPEKKVAEAEKKVEEAKKKAEDQKEEDRRNYPTNTYKTLELEIAESDVEVKKAELELVKEEAKEPRNEEKVKQAKAEVESKKAEATRLEKIKTDRKKAEEEAKRKAAEEDKVKEKPAEQPQPAPAPKAEKPAPAPKPENPAEQPKAEKPADQQAEEDYARRSEEEYNRLTQQQPPKTEKPAQP

SEQ ID NO: 14 - protein, 115 aa, Streptococcus pneumoniae
PEKKVAEAEKKVEEAKKKAEDQKEEDRRNYPTNTYKTLELEIAESDVEVKKAELELVKEEAKEPRNEEKVKQAKAEVESKKAEATRLEKIKTDRKKAEEEAKRKAAEEDKVKEKP

SEQ ID NO: 15 - protein, 13 aa, synthetic construct
  REGION 1..13: note = T cell epitope
QYIKANSKFIGIT

SEQ ID NO: 16 - protein, 15 aa, synthetic construct
  REGION 1..15: note = T cell epitope
PVVEVNGVTIQVGSR

SEQ ID NO: 17 - protein, 471 aa, synthetic construct
  REGION 1..471: note = PdB pneumolysin mutant
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAFEWWRTVYEKTDLPLVRKRTISIWGTTLYPQVEDKVEND

SEQ ID NO: 18 - protein, 529 aa, synthetic construct
  REGION 1..529: note = PdB-2TCE-linear NEEK fusion protein
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAFEWWRTVYEKTDLPLVRKRTISIWGTTLYPQVEDKVENDQYIKANSKFIGITQYIKANSKFIGITGGKAELELVKEEAKEPRNEEKVKQAKAEVESK

SEQ ID NO: 19 - protein, 559 aa, synthetic construct
  REGION 1..559: note = Looped YPT-PdB-2TCE-linear NEEK fusion protein
MACKKAEDQKEEDRRNYPTNTYKTLELECAEANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAFEWWRTVYEKTDLPLVRKRTISIWGTTLYPQVEDKVENDQYIKANSKFIGITQYIKANSKFIGITGGKCELELVKEEAKEPRNEEKVKQAKAECESK

SEQ ID NO: 20 - protein, 486 aa, synthetic construct
  REGION 1..486: note = Delta6N385-NEEK fusion protein
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIRECTGLAWEWWRTVYEKTDLPLVRKRTISIWGTTLYPQVEDKVENDKECAKEPRNEEKVKQCK

SEQ ID NO: 21 - protein, 516 aa, synthetic construct
  REGION 1..516: note = Delta6N385-TCE-NEEK fusion protein
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIRECTGLAWEWWRTVYEKTDLPLVRKRTISIWGTTLYPQVEDKVENDGGQYIKANSKFIGITQYIKANSKFIGITGGKECAKEPRNEEKVKQCK

SEQ ID NO: 22 - protein, 500 aa, synthetic construct
  REGION 1..500: note = YPT-Delta6N385 fusion protein
MACKKAEDQKEEDRRNYPTNTYKTLELECAEMANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 23 - protein, 547 aa, synthetic construct
  REGION 1..547: note = YPT-Delta6N385-TCE-NEEK fusion protein
MACKKAEDQKEEDRRNYPTNTYKTLELECAEMANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIRECTGLAWEWWRTVYEKTDLPLVRKRTISIWGTTLYPQVEDKVENDGGQYIKANSKFIGITQYIKANSKFIGITGGKECAKEPRNEEKVKQCK

SEQ ID NO: 24 - protein, 548 aa, synthetic construct
  REGION 1..548: note = YPT-L460D-TCE-NEEK fusion protein
MACKKAEDQKEEDRRNYPTNTYKTLELECAEGGANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTDYPQVEDKVENDQYIKANSKFIGITQYIKANSKFIGITGGKECAKEPRNEEKVKQCK

SEQ ID NO: 25 - protein, 488 aa, synthetic construct
  REGION 1..488: note = L460D-NEEK fusion protein
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTDYPQVEDKVENDKECAKEPRNEEKVKQCK

SEQ ID NO: 26 - DNA, 31 bp, synthetic construct
  misc_feature 1..31: note = JAT201 primer
  source 1..31: mol_type = other DNA
cgcgggatccagaagatggcaaataaagcag

SEQ ID NO: 27 - DNA, 180 bp, synthetic construct
  misc_feature 1..180: note = C-term fusion primer
  source 1..180: mol_type = other DNA
cgcggagctcagagctatttaagttgcttaactttttcctcgtttcgaggttccttagcaagtttaccaccagtaataccaataaatttagaattagctttaatatattgagtaataccaataaatttagaattagctttaatatattgaccaccgtcattttctaccttatcttctacc

SEQ ID NO: 28 - DNA, 29 bp, synthetic construct
  misc_feature 1..29: note = JAT209 primer
  source 1..29: mol_type = other DNA
cgcgcatatgaagatggcaaataaagcag

SEQ ID NO: 29 - DNA, 32 bp, synthetic construct
  misc_feature 1..32: note = JAT210 primer
  source 1..32: mol_type = other DNA
cgcgggatccagagctatttaagttgcttaac

SEQ ID NO: 30 - DNA, 127 bp, synthetic construct
  misc_feature 1..127: note = Construct 2 primer
  source 1..127: mol_type = other DNA
cgcgcatatggcttgtaaaaaagccgaggatcaaaaagaagaagatcgccgtaactacccaaccaatacttacaaaacgcttgaacttgaatgtgctgagggtggtgcaaataaagcagtaaatgac

SEQ ID NO: 31 - DNA, 35 bp, synthetic construct
  misc_feature 1..35: note = PLYNde1 primer
  source 1..35: mol_type = other DNA
gcgcgcgccatatggcaaataaagcagtaaatgac

SEQ ID NO: 32 - DNA, 87 bp, synthetic construct
  misc_feature 1..87: note = NEEK Sac1 primer
  source 1..87: mol_type = other DNA
cgcgcggagctcctatttacattgcttaactttttcctcgtttcgaggttccttagcacactctttgtcattttctaccttatcctc

SEQ ID NO: 33 - DNA, 127 bp, synthetic construct; identical to SEQ ID NO: 30
  misc_feature 1..127: note = YPT primer
  source 1..127: mol_type = other DNA

SEQ ID NO: 34 - DNA, 29 bp, synthetic construct
  misc_feature 1..29: note = JAT201b primer
  source 1..29: mol_type = other DNA
cgcgtaacatatgatggcaaataaagcag

SEQ ID NO: 35 - DNA, 181 bp, synthetic construct
  misc_feature 1..181: note = TCENEEK2 primer
  source 1..181: mol_type = other DNA
cgcggagctcctatttacattgcttaactttttcctcgtttcgaggttccttagcacactctttaccaccagtaataccaataaatttagaattagctttaatatattgaccaccagtaataccaataaatttagaattagctttaatatattgaccaccgtcattttctaccttatcctc

SEQ ID NO: 36 - DNA, 34 bp, synthetic construct
  misc_feature 1..34: note = JAT215 primer
  source 1..34: mol_type = other DNA
cgccgagctcctagtcattttctaccttatcctc

SEQ ID NO: 37 - DNA, 33 bp, synthetic construct
  misc_feature 1..33: note = YPTNDE primer
  source 1..33: mol_type = other DNA
cgcgcgcgcatatggcttgtaaaaaagccgagg

SEQ ID NO: 38 - DNA, 40 bp, synthetic construct
  misc_feature 1..40: note = NeekSac primer
  source 1..40: mol_type = other DNA
gcgcgcgagctcttactatttacattgcttaactttttcc

SEQ ID NO: 39 - protein, 471 aa, synthetic construct
  REGION 1..471: note = L460D pneumolysoid mutant
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTDYPQVEDKVEND

SEQ ID NO: 40 - protein, 470 aa, synthetic construct
  REGION 1..470: note = G293S/L460D pneumolysoid
ANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILSGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTDYPQVEDKVEND

SEQ ID NO: 41 - protein, 33 aa, synthetic construct
  REGION 1..33: note = CbpA variant sequence
MACKKAEDQKEEDRRNYPTNTYKTLELECAEGG

SEQ ID NO: 42 - protein, 17 aa, synthetic construct
  REGION 1..17: note = CbpA
KECAKEPRNEEKVKQCK

SEQ ID NO: 43 - protein, 520 aa, synthetic construct
  REGION 1..520: note = MTRV01 or CbpA-G293S/L460D pneumolysoid-CbpA
MACKKAEDQKEEDRRNYPTNTYKTLELECAEGGANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILSGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTDYPQVEDKVENDKECAKEPRNEEKVKQCK